(19) United States
(12) Patent Application Publication
(10) Pub. No.: US 2011/0268762 A1
(43) Pub. Date: Nov. 3, 2011

(54) IMMUNIZATION OF AVIANS BY MUCOSAL ADMINISTRATION OF NON-REPLICATING VECTORED VACCINES

(76) Inventors: Haroldo Toro, Auburn, AL (US); De-Chu C. Tang, Hoover, AL (US); Kent R. Van Kampen, Hoover, AL (US)

(21) Appl. No.: 13/071,787

(22) Filed: Mar. 25, 2011

Related U.S. Application Data
(63) Continuation-in-part of application No. PCT/US2009/058617, filed on Sep. 28, 2009.
(60) Provisional application No. 61/100,623, filed on Sep. 26, 2008.

(51) Int. Cl. A61K 39/145; A61K 39/235; A61K 39/255; A61K 39/215; A61K 39/15; A61K 39/275; A61K 39/17; A61K 39/21; A61K 39/29; A61P 37/04; A61P 31/12; A61P 31/16; A61P 31/14; A61P 31/22; B65D 83/14; A61K 39/00

(52) U.S. Cl. 424/205.1; 424/184.1; 424/233.1; 424/209.1; 424/229.1; 424/222.1; 424/215.1; 424/232.1; 424/214.1; 424/207.1; 424/225.1; 222/635

(57) ABSTRACT

The present invention relates generally to the fields of immunology and vaccine technology. More specifically, the invention relates to mucosal administration via aerosol spray to avians of immunogenic and vaccine compositions, including those comprising recombinant human adenovirus vectors for delivery of genes encoding avian immunogens or antigens, such as genes encoding avian influenza virus. The invention also provides methods and apparatus for use in such administration.

### CROSS-REFERENCE TO RELATED APPLICATIONS

This application is a continuation-in-part application of international patent application Serial No. PCT/US2009/058617 filed Sep. 28, 2009, which published as PCT Publication No. WO 2010/037027 on Apr. 1, 2010, which claims benefit of U.S. Provisional Application Ser. No. 61/100,623, filed Sep. 26, 2008, which is hereby incorporated by reference.

Mention is made of U.S. patent application Ser. Nos. 11/504,152 filed Aug. 15, 2006; 60/708,524, filed Aug. 15, 2005; 10/052,323, filed Jan. 18, 2002; 10/116,963, filed Apr. 5, 2002; 10/346,021, filed Jan. 16, 2003 and U.S. Pat. Nos. 6,706,693; 6,716,823; 6,348,450, and PCT/US98/16739, filed Aug. 13, 1998 which are incorporated by reference herein in their entirety.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

### FIELD OF THE INVENTION

The present invention relates generally to the fields of immunology and vaccine technology. More specifically, the invention relates to recombinant non-replicating vectors such as E1-defective human adenovirus vectors for delivery of genes encoding avian immunogens or antigens, such as avian influenza virus genes, into avians. The invention also provides methods of introducing and expressing an avian immunogen in avian subjects, including avian embryos, as well as methods of eliciting an immune response in avian subjects to immunogens, such methods including administration via mucosal routes including aerosol spray or eye drop.

### BACKGROUND OF THE INVENTION

Avian influenza (AI) is a highly contagious pathogen that infects avian species, other animals, and humans. Since 1997, there have been several incidents of transmission of AI virus to humans (Subbarao et al., 1998; Ungchusak et al., 2005). Evidence also shows that genetic recombination between avian and human influenza viruses have occurred on multiple occasions in medical history (Kawaoka et al., 1989). Because avian domestic species and humans are in close contact, it is believed that the generation of new AI virus strains that could potentially cross the species barrier into the human population will continue to be a public health concern.

Mass vaccination of avians appears to be the most promising approach to prevent dissemination of AI virus and to reduce the risk of human pandemics. Vaccination of avians with inactivated whole virus vaccines has been performed in some countries over the past several years. These AI vaccines are prepared from amnio-allantoic fluid harvested from infected eggs, and are subsequently inactivated by formalin or β-propiolactone (Tollis and Di Trani, 2002). However, the unpredictable emergence of new AI virus strains, the evolution of AI virus into a form highly lethal to chicken embryos (Wood et al., 2002), the necessity for individual parenteral delivery of inactivated vaccines, and the possible dissemination of lethal AI strains by bioterrorists make the rapid development and timely supply of safe and efficacious AI vaccine a crucial, yet very difficult, task. In addition, it is not possible to discriminate field-infected chickens from those previously vaccinated with inactivated AI viruses of the same strains (Normile, 2004).

An experimental recombinant fowlpox virus encoding the hemagglutinin (HA) of an AI virus has protected chickens against a H5N2 AI virus challenge after wing-web puncture, although the hemagglutination-inhibition (HI) serologic response was negligible (Beard et al., 1992). Chickens inoculated through the wing web with a live recombinant vaccinia virus expressing HA also developed protective immunity against a lethal AI virus challenge with low levels of serum HI antibody detected (Chambers et al., 1988). Although AI isolates of waterfowl-origin that have a tropism for the alimentary tract have been inoculated into chickens as an oral AI vaccine (Crawford et al., 1998), those isolates are not expected to be broadly effective against new AI virus strains, due to the inherently dynamic evolution of this type of virus.

Avians have also been immunized by subcutaneous injection of HA proteins expressed from baculovirus vectors (Crawford et al., 1999), and inoculation of an expression plasmid encoding HA into the skin using a gene gun (Fynan et al., 1993). These AI vaccines are able to protect avians from exhibiting clinical signs and death, and reduce respiratory and intestinal replication of a challenge virus containing homologous HA. There is also evidence that a low-cost aerosol AI vaccine expressing HA from a Newcastle disease virus vector (Swayne, 2003) or a recombinant influenza virus containing a non-pathogenic influenza virus backbone may be efficacious (Lee et al., 2004; Webby et al., 2004).

Most of the above AI vaccines rely upon labor-intensive parenteral delivery. The oral and aerosol AI vaccines suffer from inconsistencies in delivering a uniform dose to individual birds during mass-inoculation. The replicating vectors used in some vaccines also pose a biohazard by introducing unnatural microbial forms to the environment. The recombined influenza virus vaccine could even generate harmful reassortments through recombination between a reassortant influenza virus and a wild AI virus concurrently circulating in the environment (Hilleman, 2002).

There are several noteworthy reasons for utilizing recombinant Adenovirus (“Ad”) vectors as a vaccine carrier. Ad vectors are able to transduce both mitotic and postmitotic cells in situ. Additionally, preparation of Ad stocks containing high titers of virus (i.e., greater than 10¹² pfu [plaque-forming units] per ml) are easy to generate, which makes it possible to transduce cells in situ at high multiplicity of infection (MOI). Ad vectors also have a proven safety record, based on their long-term use as a vaccine. Further, the Ad vector is capable of inducing high levels of gene expression (at least as an initial burst), and replication-defective Ad vectors can be easily bioengineered, manufactured, and stored using techniques well known in the art.

Ad-based vaccines are more potent than DNA vaccines due to Ad vector's high affinity for specific receptors and its ability to escape the endosomal pathway (Curiel, 1994). Ad vectors may transduce part of a chicken embryo through binding of its fiber to the coxsackie and adenovirus receptor (CAR) found on the surface of chicken cells (Tan et al., 2001). In addition, at least one of the Ad components, hexon, is highly immunogenic and can confer adjuvant activity to exogenous antigens (Molinier-Frenkel et al., 2002).

Ad-based vaccines mimic the effects of natural infections in their ability to induce major histocompatibility complex (MHC) class I restricted T-cell responses, yet eliminate the possibility of reversion back to virulence because only a subfragment of the pathogen's genome is expressed from the vector. This “selective expression” may solve the problem of differentiating vaccinated-but-uninfected animals from their infected counterparts, because the specific markers of the pathogen not encoded by the vector can be used to discriminate the two events. Notably, propagation of the pathogen is not required for generating vectored vaccines because the relevant antigen genes can be amplified and cloned directly from field samples (Rajakumar et al., 1990). This is particularly important for vaccine production from highly virulent AI strains, such as H5N1, because these strains are too dangerous and difficult to propagate (Wood et al., 2002). In addition to the above criteria, commercial concerns factor heavily in the poultry industry. The current AI vaccine alone costs about 7 cents per bird, not counting the labor of injecting running birds (Normile, 2004).

Replication-incompetent E1/E3-defective human Ad serotype 5 (Ad5)-derived vectors have been extensively studied in mammals (Graham and Prevec, 1995). Although chickens have been immunized by subcutaneous or intradermal injection of an avian Ad chicken embryo lethal orphan (CELO) viral vector encoding an antigen (Francois et al., 2004), the CELO vector has a low compliance rate and could be potentially harmful due to its ability to replicate in chicken cells. Since CELO possesses no identifiable E1, E3, and E4 regions (Chiocca et al., 1996), a replication-incompetent CELO vector is not available as a carrier for immunization at this time. The present invention addresses this need by providing a safe and efficient method for gene delivery to protect avians in a wide variety of disease settings, and consequently prevent transmission of avian pathogens to humans.

The Harderian gland (glandula lacrimalis accesoria) (HG) was first described in 1694 by Johann Jacob Harder (1656-1711) and is found in most terrestrial vertebrates (Payne, 1994). The HG is a tubulo-alveolar gland located within the eye sockets posterior to the eyeball in chickens and its secretory duct is morphologically distinct after leaving the body of the gland to open onto the surface of the nictitating membrane (Payne, 1994). The functions of the gland are diverse and include a) lubrication of the eye and nictitating membrane, b) immune responses in birds, c) photoreception in rodents, d) forms part of the retinal-pineal axis, e) production of pheromones, f) thermoregulatory lipid production in rodents, g) osmoregulation in some reptiles, h) production of growth factors and i) saliva production in some chelonians (Payne 1994; Chieffi 1996). The HGs are perfectly located to generate adaptive immune responses upon ocular exposure to microbial pathogens or vectored vaccines.

Chickens have been previously immunized against avian pathogens by intramuscular injection (Gao, 2006) or in ovo administration (Toro, 2007) of a human Ad5-vectored vaccine. Further study was needed to evaluate the ability of the HGs to generate adaptive mucosal immunity in chickens, as is described here. Previous studies with RCA-free human Ad5 vector expressing a codon-optimized H5 HA gene of A/turkey/Wisconsin/68 (AdTW68.H5_{ck}) induced protective immunity against challenge with highly pathogenic AI (HPAI) H5N2 A/chicken/Quer/95 and H5N1 A/swan/Mongolia/244L/2005 strains following a single in ovo immunization (Toro, 2007). In order to protect existing chicken populations from AI, alternative immunization protocols to in ovo immunization are required. Since influenza virus is transmitted following exposure to mucosal surfaces, the ability of this AdTW68.H5_{ck} vector to induce mucosal and systemic immunity to H5 HA in chickens after ocular application was tested. The AdTW68.H5_{ck} vector may induce mucosal as well as systemic immunity in chickens following HG-associated ocular immunization. It is conceivable that the HGs may be an immunocompetent tissue capable of triggering an immune response as mucosal effector sites, based on previous findings (Toro, 1996). In addition, the observation that the J-chain is expressed in HG's B cells further corroborates HG as a key tissue in defense mechanisms. The chicken J-chain gene displayed a high degree of homology with that of other species, and is expressed at an early stage of development of the chicken immune system (Takahashi 2000). Furthermore, the J-chain played an important role in polymerization of IgA and IgM and their transport across the mucosal epithelium and thus will be a requirement for transport of polymeric IgA (pIgA) across a mucosal epithelium (Johansen 2000). The polymeric immunoglobulin receptor (pIgR) of chicken (Gallus gallus) was recently cloned (Wieland 2004) and confirmed the conservation of this mucosal transport system in avian species and its importance in protective mucosal immunity.

Citation or identification of any document in this application is not an admission that such document is available as prior art to the present invention.

### SUMMARY OF THE INVENTION

It has now been surprisingly shown that human adenovirus-vectored vaccines can rapidly, safely, and effectively immunize avians, including via in ovo delivery and aerosol spray. Mass immunization of avians against several avian pathogens is crucial to prevent enormous economic loss, and to impede transmission of avian pathogens, such as avian influenza virus, to the human population. In ovo delivery of vaccines or immunogenic compositions with a mechanized injector is a non-labor-intensive method for mass immunization of avians in a timely manner. Alternatively, mucosal (ocular or aerosol spray) delivery of vaccines or immunogenic compositions is routinely performed in the poultry industry. In addition coarse spray delivery is a non-labor-intensive method for mass immunization of avians in a timely manner. Vaccination via mucosal routes not only elicits a systemic immune response but also an immune response at the port of entrance of the pathogen. Unlike other avian vaccines, production of human adenovirus-vectored vaccines or immunogenic compositions does not require the propagation of lethal pathogens and does not involve transmission of antigens or immunogens by a vector that is capable of replicating in avians. Furthermore, immunization by these types of vaccine or immunogenic composition allows differentiation between vaccinated and naturally infected animals.

In one embodiment, the present invention provides a recombinant human adenovirus expression vector that may comprise and express an adenoviral DNA sequence, and a promoter sequence operably linked to a foreign sequence encoding one or more avian antigens or immunogens of interest.

In another embodiment, the human adenoviral sequences may be derived from human adenovirus serotype 5. The human adenoviral sequences may be derived from replication-defective adenovirus, non-replicating adenovirus, replication-competent adenovirus, or wild-type adenovirus.

The promoter sequence may be selected from the group consisting of viral promoters, avian promoters, CMV promoter, SV40 promoter, β-actin promoter, albumin promoter, EF1-α promoter, PγK promoter, MFG promoter, and Rous sarcoma virus promoter.

The one or more avian antigens or immunogens of interest may be derived from, for example, avian influenza virus, infectious bursal disease virus, Marek's disease virus, Herpesviruses such as infectious laryngotracheitis virus, avian infectious bronchitis virus, avian reovirus, poxviruses including avipox, fowlpox, canarypox, pigeonpox, quailpox, and dovepox, avian polyomavirus, Newcastle Disease virus, avian pneumovirus, avian rhinotracheitis virus, avian reticuloendotheliosis virus, avian retroviruses, avian endogenous virus, avian erythroblastosis virus, avian hepatitis virus, avian anemia virus, avian enteritis virus, Pacheco's disease virus, avian leukemia virus, avian parvovirus, avian rotavirus, avian leukosis virus, avian musculoaponeurotic fibrosarcoma virus, avian myeloblastosis virus, avian myeloblastosis-associated virus, avian myelocytomatosis virus, avian sarcoma virus, or avian spleen necrosis virus.

In one embodiment, the one or more avian antigens or immunogens of interest may be derived from avian influenza, i.e., hemagglutinin, nucleoprotein, matrix, and neuraminidase.

In yet another embodiment, the one or more avian antigens or immunogens of interest may be derived from hemagglutinin subtype 3, 5, 7, or 9.

Another embodiment of the invention provides an immunogenic composition or vaccine for in vivo delivery into an avian subject comprising a veterinarily acceptable vehicle or excipient and a recombinant human adenovirus expression vector that may comprise and express an adenoviral DNA sequence, and a promoter sequence operably linked to a foreign sequence encoding one or more avian antigens or immunogens of interest.

In one embodiment, the adenoviral DNA sequence may be derived from adenovirus serotype 5 (Ad5).

In another embodiment, the human adenoviral sequences may be derived from human adenovirus serotype 5. The human adenoviral sequences may be derived from replication-defective adenovirus.

The promoter sequence may be selected from the group consisting of viral promoters, avian promoters, CMV promoter, SV40 promoter, β-actin promoter, albumin promoter, EF1-α promoter, PγK promoter, MFG promoter, and Rous sarcoma virus promoter.

The one or more avian antigens or immunogens of interest may be derived from, for example, avian influenza virus, infectious bursal disease virus, Marek's disease virus, Herpesviruses such as infectious laryngotracheitis virus, avian infectious bronchitis virus, avian reovirus, poxviruses including avipox, fowlpox, canarypox, pigeonpox, quailpox, and dovepox, avian polyomavirus, Newcastle Disease virus, avian pneumovirus, avian rhinotracheitis virus, avian reticuloendotheliosis virus, avian retroviruses, avian endogenous virus, avian erythroblastosis virus, avian hepatitis virus, avian anemia virus, avian enteritis virus, Pacheco's disease virus, avian leukemia virus, avian parvovirus, avian rotavirus, avian leukosis virus, avian musculoaponeurotic fibrosarcoma virus, avian myeloblastosis virus, avian myeloblastosis-associated virus, avian myelocytomatosis virus, avian sarcoma virus, or avian spleen necrosis virus.

The one or more avian antigens or immunogens of interest may derived from avian influenza, i.e., hemagglutinin, nucleoprotein, matrix, and neuraminidase.

The one or more avian antigens or immunogens of interest may be derived from hemagglutinin subtype 3, 5, 7, or 9.

The immunogenic composition or vaccine may further comprise an adjuvant.

The immunogenic composition or vaccine may further comprise an additional vaccine.

Another embodiment of the invention provides a method of introducing and expressing one or more avian antigens or immunogens in a cell, comprising contacting the cell with a recombinant human adenovirus expression vector that comprises and expresses an adenoviral DNA sequence, and a promoter sequence operably linked to a foreign sequence encoding one or more avian antigens or immunogens of interest, and culturing the cell under conditions sufficient to express the one or more avian antigens or immunogens in the cell.

The cell may be a 293 cell or a PER.C6 cell.

The one or more avian antigens or immunogens of interest may be derived from avian influenza virus, infectious bursal disease virus, Marek's disease virus, avian herpesvirus, infectious laryngotracheitis virus, avian infectious bronchitis virus, avian reovirus, avipox, fowlpox, canarypox, pigeonpox, quailpox, and dovepox, avian polyomavirus, Newcastle Disease virus, avian pneumovirus, avian rhinotracheitis virus, avian reticuloendotheliosis virus, avian retroviruses, avian endogenous virus, avian erythroblastosis virus, avian hepatitis virus, avian anemia virus, avian enteritis virus, Pacheco's disease virus, avian leukemia virus, avian parvovirus, avian rotavirus, avian leukosis virus, avian musculoaponeurotic fibrosarcoma virus, avian myeloblastosis virus, avian myeloblastosis-associated virus, avian myelocytomatosis virus, avian sarcoma virus, or avian spleen necrosis virus.

In one embodiment, the foreign sequence encoding the one or more avian antigens or immunogens of interest may be derived from one or more avian viruses.

In another embodiment, the foreign sequence encoding the one or more avian antigens or immunogens of interest may derived from avian influenza.

In yet another embodiment, the foreign sequence encoding the one or more avian antigens or immunogens of interest may be selected from the group consisting of hemagglutinin, nucleoprotein, matrix, or neuraminidase

In a further embodiment, the foreign sequence encoding the one or more avian antigens or immunogens of interest may be selected from the group consisting of hemagglutinin subtype 3, 5, 7, or 9.

Another embodiment of the invention provides a method of introducing and expressing one or more avian influenza antigens or immunogens in an avian embryo, comprising contacting the avian embryo with a recombinant human adenovirus expression vector that comprises and expresses an adenoviral DNA sequence, and a promoter sequence operably linked to a foreign sequence encoding one or more avian antigens or immunogens of interest, thereby obtaining expression of the one or more avian influenza antigens or immunogens in the avian embryo.

In one embodiment, the foreign sequence encoding the one or more avian antigens or immunogens of interest may be derived from one or more avian viruses.

In another embodiment, the foreign sequence encoding the one or more avian antigens or immunogens of interest may be derived from avian influenza virus.

In yet another embodiment, the foreign sequence encoding the one or more avian antigens or immunogens of interest may be selected from the group consisting of genes encoding hemagglutinin, spike protein, other external proteins, nucleoprotein, matrix, neuraminidase, and non-structural proteins such as enzymes or other regulatory proteins.

In still another embodiment, the foreign sequence encoding the one or more avian antigens or immunogens of interest may be selected from the group consisting of hemagglutinin subtype 3, 5, 7, or 9.

The method of introducing and expressing one or more avian influenza antigens or immunogens in an avian embryo may occur by in ovo delivery.

In a further embodiment of the invention, the method of introducing and expressing one or more avian influenza antigens or immunogens in an avian embryo preferably may occur by aerosol spray delivery.

Another embodiment of the present invention provides a method of eliciting an immunogenic response in an avian subject, comprising administering an immunologically effective amount of the composition of the invention to the avian subject.

Yet another embodiment of the present invention provides a method of eliciting an immunogenic response in an avian subject, comprising infecting the avian subject with an immunologically effective amount of an immunogenic composition comprising a recombinant human adenovirus expression vector that comprises and expresses an adenoviral DNA sequence, and a promoter sequence operably linked to a foreign sequence encoding one or more avian antigens or immunogens of interest, wherein the one or more avian antigens or immunogens of interest are expressed at a level sufficient to elicit an immunogenic response to the one or more avian antigens or immunogens of interest in the avian subject.

The one or more avian antigens or immunogens of interest may be derived from avian influenza virus, infectious bursal disease virus, Marek's disease virus, avian herpesvirus, infectious laryngotracheitis virus, avian infectious bronchitis virus, avian reovirus, avipox, fowlpox, canarypox, pigeonpox, quailpox, and dovepox, avian polyomavirus, Newcastle Disease virus, avian pneumovirus, avian rhinotracheitis virus, avian reticuloendotheliosis virus, avian retroviruses, avian endogenous virus, avian erythroblastosis virus, avian hepatitis virus, avian anemia virus, avian enteritis virus, Pacheco's disease virus, avian leukemia virus, avian parvovirus, avian rotavirus, avian leukosis virus, avian musculoaponeurotic fibrosarcoma virus, avian myeloblastosis virus, avian myeloblastosis-associated virus, avian myelocytomatosis virus, avian sarcoma virus, or avian spleen necrosis virus.

In one embodiment, the foreign sequence encoding the one or more avian antigens or immunogens of interest may be derived from avian influenza.

In another embodiment, the foreign sequence encoding the one or more avian antigens or immunogens of interest may be selected from the group consisting of hemagglutinin, nucleoprotein, matrix, or neuraminidase.

In yet another embodiment, the foreign sequence encoding the one or more avian antigens or immunogens of interest may be selected from the group consisting of hemagglutinin subtype 3, 5, 7, or 9.

The method may further comprise administering an additional vaccine.

In one embodiment, the method of infecting may occur by in ovo delivery.

Alternatively, the method of infecting may occur by aerosol spray delivery.

Another embodiment of the invention provides a method of eliciting an immunogenic response in an avian subject, comprising infecting the avian subject with an immunologically effective amount of an immunogenic composition comprising a recombinant human adenovirus expression vector that comprises and expresses an adenoviral DNA sequence, and a promoter sequence operably linked to a foreign sequence encoding one or more avian antigens or immunogens of interest, wherein the one or more avian antigens or immunogens of interest are expressed at a level sufficient to elicit an immunogenic response to the one or more avian antigens or immunogens of interest in the avian subject.

The one or more avian antigens or immunogens of interest may be derived from avian influenza virus, infectious bursal disease virus, Marek's disease virus, avian herpesvirus, infectious laryngotracheitis virus, avian infectious bronchitis virus, avian reovirus, avipox, fowlpox, canarypox, pigeonpox, quailpox, and dovepox, avian polyomavirus, Newcastle Disease virus, avian pneumovirus, avian rhinotracheitis virus, avian reticuloendotheliosis virus, avian retroviruses, avian endogenous virus, avian erythroblastosis virus, avian hepatitis virus, avian anemia virus, avian enteritis virus, Pacheco's disease virus, avian leukemia virus, avian parvovirus, avian rotavirus, avian leukosis virus, avian musculoaponeurotic fibrosarcoma virus, avian myeloblastosis virus, avian myeloblastosis-associated virus, avian myelocytomatosis virus, avian sarcoma virus, or avian spleen necrosis virus.

In one embodiment, the foreign sequence encoding the one or more avian antigens or immunogens of interest may be derived from avian influenza.

In another embodiment, the foreign sequence encoding the one or more avian antigens or immunogens of interest may be selected from the group consisting of hemagglutinin, nucleoprotein, matrix, or neuraminidase.

In yet another embodiment, the foreign sequence encoding the one or more avian antigens or immunogens of interest may be selected from the group consisting of hemagglutinin subtype 3, 5, 7, or 9.

The method may further comprise administering an additional vaccine.

The avian subject may be infected by intramuscular injection of the wing-web, wing-tip, pectoral muscle, or thigh musculature.

The avian subject may also be infected in ovo or by aerosol spray.

Another embodiment of the invention provides a method for inoculation of an avian subject, comprising in ovo or aerosol spray administration of a recombinant human adenovirus containing and expressing a heterologous nucleic acid molecule encoding an antigen of a pathogen of the avian subject.

The human adenovirus may comprise sequences derived from adenovirus serotype 5.

In one embodiment, the human adenovirus may comprise sequences derived from replication-defective adenovirus, non-replicating adenovirus, replication-competent adenovirus, or wild-type adenovirus.

In one embodiment, the antigen of a pathogen of the avian is derived from avian influenza virus, infectious bursal disease virus, Marek's disease virus, avian herpesvirus, infectious laryngotracheitis virus, avian infectious bronchitis virus, avian reovirus, avipox, fowlpox, canarypox, pigeonpox, quailpox, and dovepox, avian polyomavirus, Newcastle Disease virus, avian pneumovirus, avian rhinotracheitis virus, avian reticuloendotheliosis virus, avian retroviruses, avian endogenous virus, avian erythroblastosis virus, avian hepatitis virus, avian anemia virus, avian enteritis virus, Pacheco's disease virus, avian leukemia virus, avian parvovirus, avian rotavirus, avian leukosis virus, avian musculoaponeurotic fibrosarcoma virus, avian myeloblastosis virus, avian myeloblastosis-associated virus, avian myelocytomatosis virus, avian sarcoma virus, or avian spleen necrosis virus.

In another embodiment, the antigen of a pathogen of the avian may be derived from avian influenza.

In yet another embodiment, the avian influenza antigens or immunogens may be selected from the group consisting of hemagglutinin, nucleoprotein, matrix, or neuraminidase.

In still another embodiment, the avian influenza antigens or immunogens may be selected from the group consisting of hemagglutinin subtype 3, 5, 7, or 9.

The method may further comprise administering an additional vaccine.

Another embodiment of the invention provides an in ovo administration apparatus for delivery of an immunogenic composition to an avian embryo wherein the apparatus contains a recombinant human adenovirus expression vector expressing one or more avian antigens or immunogens of interest, wherein the apparatus delivers to the recombinant human adenovirus to the avian embryo.

A further embodiment of the invention provides an aerosol spray administration apparatus for delivery of an immunogenic composition to one or more avians wherein the apparatus contains a recombinant human adenovirus expression vector expressing one or more avian antigens or immunogens of interest, wherein the apparatus delivers the recombinant human adenovirus to the one or more avians.

In one embodiment, the human adenovirus expression vector may comprise sequences derived from adenovirus serotype 5.

In another embodiment, the human adenovirus expression vector may comprise sequences derived from replication-defective adenovirus, non-replicating human adenovirus, replication-competent adenovirus, or wild-type adenovirus.

In one embodiment, the one or more avian antigens or immunogens of interest are derived from avian influenza virus, infectious bursal disease virus, Marek's disease virus, avian herpesvirus, infectious laryngotracheitis virus, avian infectious bronchitis virus, avian reovirus, avipox, fowlpox, canarypox, pigeonpox, quailpox, and dovepox, avian polyomavirus, Newcastle Disease virus, avian pneumovirus, avian rhinotracheitis virus, avian reticuloendotheliosis virus, avian retroviruses, avian endogenous virus, avian erythroblastosis virus, avian hepatitis virus, avian anemia virus, avian enteritis virus, Pacheco's disease virus, avian leukemia virus, avian parvovirus, avian rotavirus, avian leukosis virus, avian musculoaponeurotic fibrosarcoma virus, avian myeloblastosis virus, avian myeloblastosis-associated virus, avian myelocytomatosis virus, avian sarcoma virus, or avian spleen necrosis virus.

In another embodiment, the one or more avian antigens or immunogens of interest may be derived from avian influenza.

In yet another embodiment, the avian influenza antigens or immunogens may be selected from the group consisting of hemagglutinin, nucleoprotein, matrix, or neuraminidase.

In still another embodiment, the avian influenza antigens or immunogens of interest may be selected from the group consisting of hemagglutinin subtype 3, 5, 7, or 9.

The method may further comprise administering an additional vaccine.

Accordingly, it is an object of the invention to not encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. §112, first paragraph) or the EPO (Article 83 of the EPC), such that Applicants reserve the right and hereby disclose a disclaimer of any previously described product, process of making the product, or method of using the product.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The following Detailed Description, given by way of example, but not intended to limit the invention to specific embodiments described, may be understood in conjunction with the accompanying Figures, incorporated herein by reference, in which:

FIG. 1 is a graph depicting the immunization of chickens by in ovo and intramuscular injection of the recombinant adenovirus vector expressing avian influenza HA. Group 1 represents 9-day-old embryonated chicken eggs and Group 2 represents 18-day-old embryonated chicken eggs, respectively, in a volume of 200 μl at a dose of 5×10¹⁰ pfu per egg. In Group 3, the recombinant adenovirus vector expressing avian influenza HA was injected intramuscularly into three 4-week-old chickens in a volume of 100 μpi at a dose of 2.5×10¹⁰ pfu per animal.

FIG. 2 is a graph depicting the hemagglutination inhibition antibody titers (dots) detected in 28-day-old SPF chickens vaccinated with AdTW68.H5 in ovo only at days 10 or 18 of incubation, and chickens that were vaccinated in ovo at days 10 or 18 of incubation and were boosted by the nasal route at day 15 post-hatch. Bar, geometric mean log₂[HI titer]. No HI titers were detected in naive control chickens (data not shown).

FIG. 3 is a graph depicting the hemagglutination inhibition antibodies in SPF chickens at days 23 and 29 post-hatch either vaccinated in ovo only (7 chicks) at day 18 of embryonation or vaccinated in ovo and boosted intranasally at day 15 post-hatch (12 chicks) with AdTW68.H5. D23 and D29, HI titers at days 23 and 29 post-hatch, respectively; dots, log₂ [HI titer] in individual birds; bar, geometric mean log ₂[HI titer]. No HI titers were detected in 11 naïve control chickens at days 23 and 29 post-hatch (data not shown).

FIG. 4 is a graph depicting in ovo vaccination of day-18 white leghorn chicken embryos. In ovo vaccination was performed using 10¹¹ vp of AdTW68.H5 (in ovo). In a separate group, in ovo-vaccinated birds were boosted at day 15 post-hatch by intranasal instillation of AdTW68.H5 with the same dose (in ovo+nasal booster). Naïve embryos without immunization served as negative controls (Control). On day 34 of age chickens were intranasally challenged through the choanal slit with a lethal dose of the highly pathogenic A/Ck/Queretaro/14588-19/95 (H5N2) AI virus strain. Statistically significant changes in survival were determined throughout the study using the Logrank test (Prism 4.03, GraphPad Software). In ovo vaccination with AdTW68.H5 with or without nasal booster applications significantly protected chickens (100%) against a lethal challenge with AI virus, when compared to unvaccinated controls (P<0.001).

FIGS. 5A, 5B, and 5C are the graphs depicting A/chicken/Queretaro/14588-19/95 viral RNA quantitated by quantitative real-time RT-PCR (16) in oro-pharyngeal samples from vaccinated and control chickens after intranasal challenge with this highly pathogenic AI virus. Chickens were vaccinated as described in the description of FIG. 3. Samples were collected at 2 days (FIG. 5A), 4 days (FIG. 5B), and 7 days (FIG. 5C) post-infection. A significant difference (P<0.05) in viral load was achieved at day 7 between vaccinated and unvaccinated controls.

FIG. 6 is a graph depicting in ovo vaccination on day-18 performed by inoculation of the AdTW68.H5 vector at a dose of 3×10⁸ ifu. The Ad5 vector was purified by the Sartobind Q5 membrane (Sartorius North America, Inc., Edgewood, N.Y.) and resuspended in A195 buffer (Evans, 2004). Pre-challenge serum HI antibodies on D25 were analyzed. Minus sign (−) indicates birds that succumbed to challenge; plus sign (+) indicates birds that survived challenge. Survivors had significantly elevated pre-challenge serum HI activity (P<0.001) as compared to those that died (unpaired t-test; Prism 4.03). All naïve control birds and birds immunized by the control vector AdCMV-tetC produced no measurable HI antibody titers.

FIG. 7 is a graph depicting in ovo vaccination on day-18 performed by inoculation of the AdTW68.H5 vector at a dose of 3×10⁸ ifu. The Ad5 vector was purified by the Sartobind Q5 membrane (Sartorius North America, Inc., Edgewood, N.Y.) and resuspended in A195 buffer (Evans, 2004). Control and immunized birds were challenged by intranasal administration of 10⁵ EID₅₀ of the H5N1 AI virus A/swan/Mongolia//244L/2005 on D31.

FIG. 8 is a graph depicting the IgA anti-H7 present in the lachrymal fluid of chickens following aerosol spray vaccination at one day of age. Group A—aerosol spray of AdChNY94.H7 in a volume of 8 ml containing 1.1×10¹⁰ infectious units (ifu) per ml in a single dose; Group B—aerosol spray of the same vaccine in a volume of 24 ml (also 1.1×10¹⁰ ifu of Ad5 per ml) and a booster application on day 16 of age; Group C—unvaccinated control.

FIG. 9 is a graph depicting hemagglutination-inhibition (HI) antibody titers in chicken sera. Group A—aerosol spray of AdChNY94.H7 in a volume of 8 ml containing 1.1×10¹⁰ infectious units (ifu) per ml in a single dose; Group B—aerosol spray of the same vaccine in a volume of 24 ml (also 1.1×10¹⁰ ifu of Ad5 per ml) and a booster application on day 16 of age; Group C—unvaccinated control.

FIG. 10. Expression of avian influenza hemagglutinin in chicken harderian glands nine days after ocular Ad5-H5 exposure. White leghorns X days of age were exposed to 2.5×10⁸ infectious units of adenovirus expressing the AI hemagglutinin gene serotype 5. The tissues were fixed in acidic acetate-alcohol for two hours followed by incubation in sucrose (30%) after which the tissues were embedded in Neg 50 medium and frozen. Five μm sections were cut on a cryostat and were placed on slides to dry. The slides were treated with acetone, blocked with 10% FCS for 1 hr prior to staining for 4 hrs with anti-H5 affinity-purified rabbit-anti H5 antibodies. The slides were extensively washed and cover glasses were mounted with Vectashield Hard Set mounting medium. The Ad5-H5 exposed HDGLs but not the controls expressed H5.

FIG. 11. Hemagglutination-inhibition (HI) titer in plasma of chickens ocular immunized with Ad5-H5. Plasma samples were collected from control chickens (n=11) and chickens immunized two (n=15) or three (n=9) times with 2.5×10⁸ i.f.u. of Ad5-H5. HI titer was determined using 4 hemagglutinating units of a low pathogenic A/turkey/Wisconsin/68 (H5N9) strain. Titers of <1.0 Log₂ were arbitrarily assigned a titer of 0. No HI titer was detected in control chickens while the HI titer increased with each Ad5-H5 administration tested 14 days after ocular administration.

FIG. 12. Induction of Ad5-specific antibodies after ocular administration of Ad5-H5. Ad5-specific antibodies were detected by ELISA as described. In brief, the wells were coated with killed wildtype Ad5 virus, blocked and serial two-fold dilutions of the samples were incubated overnight. HRP-conjugated IgA and IgG chicken-specific antibodies were used to detect Ad5-specific antibodies. The reaction was stopped after 30 minutes at room temperature and the absorption at 405 nm was measured. The highest dilution with an OD₄₀₅ of 0.100 or more above background was defined as the endpoint-titer. No IgG or IgA antibody levels were detected in tears and serum from control chickens.

FIG. 13. IgA secreting cells in the Harderian glands after ocular immunization. Lymphocytes isolated from the Harderian glands 9 days after immunization were isolated and loaded on antigen-coated ELISPOT plates and were incubated overnight in a CO₂ incubator. Antibody secreting cells were detected with anti-IgA antibodies conjugated to HRP after which substrate was added to visualize the spots as described in the Material and Methods. Illustrated is a single well containing Harderian gland lymphocytes derived from control or ocular challenged chickens.

FIG. 14. H5- and Ad5-specific IgG antibody secreting cells in the Harderian glands after ocular immunization with Ad5-H5. Lymphocytes were isolated from the Harderian glands (HDGL) at various days after Ad5-H5 administration and were analyzed for antibody secreting cells for H5 and Ad5. Indicated are the mean numbers of IgG spot-forming cells per 10⁶ lymphocytes and one standard error. Both Ad5-specific and H5-specific IgG antibodies are produced in the Harderian glands after ocular Ad5-H5 administration.

FIG. 15. H5- and Ad5-specific IgA antibody secreting cells in the Harderian glands after ocular immunization with Ad5-H5. Lymphocytes were isolated from the Harderian glands at various days after Ad5-H5 administration and were analyzed for IgA antibody secreting cells specific for H5 and Ad5. Indicated are the mean numbers of IgA spot-forming cells per 10⁶ lymphocytes and one standard error. Both Ad5-specific and H5-specific IgA antibodies are produced in the Harderian glands and peak on day 9 and 11 after Ad5-H5 administration.

FIG. 16. Polymeric-Ig receptor expression in the Harderian glands of chickens. Total RNA was isolated from the Harderian glands of three chickens using Tri-reagent (Molecular Research, Inc.) according to manufacturer's protocols. One microgram of total RNA was reverse transcribed and amplified by 35 PCR cycles of 94° C., 1 min., 58° C., 1 min. The RT-PCR products and a 100 bp DNA ladder were separated on a 1.5% agarose gel and stained with ethidium bromide. A 400 bp amplicon was observed confirming that pIgR mRNA is produced in the Harderian glands.

FIG. 17. Immunoprecipitation of chicken IgA in tears and serum. Tears and serum were incubated overnight at 4° C. with biotinylated mouse-anti-chicken IgA monoclonal antibody or biotinylated polyclonal goat-anti-chicken IgA. The next day washed Streptavidin-conjugated Sepharose beads were added overnight at 4° C. under continuously agitation. The next day the beads were washed and boiled for 10 minutes at 100° C. in a SDS sample buffer. The immunoprecipitants were analyzed by SDS-polyacrylamide gel electrophoresis (SDS-PAGE) on a 4-12% pre-cast gradient gel. The proteins were visualized using the Silver SNAP® silverstain kit according to manufacturer protocols. Abbreviations used: monomeric IgA=mIgA, polymeric IgA=pIgA, tetrameric IgA=tIgA.

### DETAILED DESCRIPTION OF THE INVENTION

The term “nucleic acid” or “nucleic acid sequence” refers to a deoxyribonucleic or ribonucleic oligonucleotide in either single- or double-stranded form. The term encompasses nucleic acids, i.e., oligonucleotides, containing known analogues of natural nucleotides. The term also encompasses nucleic-acid-like structures with synthetic backbones, see e.g., Eckstein, 1991; Baserga et al., 1992; Milligan, 1993; WO 97/03211; WO 96/39154; Mata, 1997; Strauss-Soukup, 1997; and Samstag, 1996.

As used herein, “recombinant” refers to a polynucleotide synthesized or otherwise manipulated in vitro (e.g., “recombinant polynucleotide”), to methods of using recombinant polynucleotides to produce gene products in cells or other biological systems, or to a polypeptide (“recombinant protein”) encoded by a recombinant polynucleotide. “Recombinant means” also encompass the ligation of nucleic acids having various coding regions or domains or promoter sequences from different sources into an expression cassette or vector for expression of, e.g., inducible or constitutive expression of polypeptide coding sequences in the vectors of invention.

The term “heterologous” when used with reference to a nucleic acid, indicates that the nucleic acid is in a cell or a virus where it is not normally found in nature; or, comprises two or more subsequences that are not found in the same relationship to each other as normally found in nature, or is recombinantly engineered so that its level of expression, or physical relationship to other nucleic acids or other molecules in a cell, or structure, is not normally found in nature. A similar term used in this context is “exogenous”. For instance, a heterologous nucleic acid is typically recombinantly produced, having two or more sequences from unrelated genes arranged in a manner not found in nature; e.g., a human gene operably linked to a promoter sequence inserted into an adenovirus-based vector of the invention. As an example, a heterologous nucleic acid of interest can encode an immunogenic gene product, wherein the adenovirus is administered therapeutically or prophylactically as a vaccine or vaccine composition. Heterologous sequences can comprise various combinations of promoters and sequences, examples of which are described in detail herein.

An “antigen” is a substance that is recognized by the immune system and induces an immune response. A similar term used in this context is “immunogen”.

An “avian subject” in the context of the present invention refers to any and all domestic and wild members of the class Aves, which include, but are not limited to, Neognathae and Palaeognathae. The Neognathae order includes, among others, Anseriformes, Apodiformes, Buceroformes, Caprimulgiformes, Charadriiformes, Ciconiiformes, Coliiformes, Columbiformes, Coraciiformes, Cuculiformes, Falconiformes, Galbuliformes, Galliformes, Gaviiformes, Gruiformes, Musophagiformes, Opisthocomiformes, Passeriformes, Pelecaniformes, Phoenicopteriformes, Piciformes, Podicipediformes, Procellariiformes, Psittaciformes, Sphenisciformes, Strigiformes, Trochiliformes, Trogoniformes, Turniciformes, and Upupiformes. The Palaeognathae order includes, among others, Apterygiformes, Casuariiformes, Dinornithiformes, Rheiformes, Struthioniformes, and Tiniamiformes. Avian subjects can comprise adult avians, avian chicks, and avian embryos/eggs.

“Expression” of a gene or nucleic acid encompasses not only cellular gene expression, but also the transcription and translation of nucleic acid(s) in cloning systems and in any other context.

As used herein, a “vector” is a tool that allows or facilitates the transfer of an entity from one environment to another. By way of example, some vectors used in recombinant DNA techniques allow entities, such as a segment of DNA (such as a heterologous DNA segment, such as a heterologous cDNA segment), to be transferred into a target cell. The present invention comprehends recombinant vectors that can include viral vectors, bacterial vectors, protozoan vectors, DNA vectors, or recombinants thereof.

With respect to exogenous DNA for expression in a vector (e.g., encoding an epitope of interest and/or an antigen and/or a therapeutic) and documents providing such exogenous DNA, as well as with respect to the expression of transcription and/or translation factors for enhancing expression of nucleic acid molecules, and as to terms such as “epitope of interest”, “therapeutic”, “immune response”, “immunological response”, “protective immune response”, “immunological composition”, “immunogenic composition”, and “vaccine composition”, inter alia, reference is made to U.S. Pat. No. 5,990,091 issued Nov. 23, 1999, and WO 98/00166 and WO 99/60164, and the documents cited therein and the documents of record in the prosecution of that patent and those PCT applications; all of which are incorporated herein by reference. Thus, U.S. Pat. No. 5,990,091 and WO 98/00166 and WO 99/60164 and documents cited therein and documents of record in the prosecution of that patent and those PCT applications, and other documents cited herein or otherwise incorporated herein by reference, can be consulted in the practice of this invention; and, all exogenous nucleic acid molecules, promoters, and vectors cited therein can be used in the practice of this invention. In this regard, mention is also made of U.S. Pat. Nos. 6,706,693; 6,716,823; 6,348,450; U.S. patent application Ser. Nos. 10/424,409; 10/052,323; 10/116,963; 10/346,021; and WO 99/08713, published Feb. 25, 1999, from PCT/US98/16739.

As used herein, the terms “immunogenic composition” and “immunological composition” and “immunogenic or immunological composition” cover any composition that elicits an immune response against the antigen or immunogen of interest expressed from the adenoviral vectors and viruses of the invention; for instance, after administration into a subject, elicits an immune response against the targeted immunogen or antigen of interest. The terms “vaccinal composition” and “vaccine” and “vaccine composition” covers any composition that induces a protective immune response against the antigen(s) of interest, or which efficaciously protects against the antigen; for instance, after administration or injection into the subject, elicits an protective immune response against the targeted antigen or immunogen or provides efficacious protection against the antigen or immunogen expressed from the inventive adenovirus vectors of the invention. The term “veterinary composition” means any composition comprising a vector for veterinary use expressing a therapeutic protein as, for example, erythropoietin (EPO) or an immunomodulatory protein, such as, for example, interferon (IFN). Similarly, the term “pharmaceutical composition” means any composition comprising a vector for expressing a therapeutic protein.

An “immunologically effective amount” is an amount or concentration of the recombinant vector encoding the gene of interest, that, when administered to a subject, produces an immune response to the gene product of interest.

A “circulating recombinant form” refers to recombinant viruses that have undergone genetic reassortment among two or more subtypes or strains. Other terms used in the context of the present invention are “hybrid form”, “recombined form”, and “reassortant form”.

“Clinical isolates” refer to, for example, frequently used laboratory strains of viruses that are isolated from infected subjects and are reasserted in laboratory cells or subjects with laboratory-adapted master strains of high-growth donor viruses.

“Field isolates” refer to viruses that are isolated from infected subjects or from the environment.

The methods of the invention can be appropriately applied to prevent diseases as prophylactic vaccination or provide relief against symptoms of disease as therapeutic vaccination.

The recombinant vectors of the present invention can be administered to a subject either alone or as part of an immunological or immunogenic composition. The recombinant vectors of the invention can also be used to deliver or administer one or more proteins to a subject of interest by in vivo expression of the protein(s).

It is noted that immunological products and/or antibodies and/or expressed products obtained in accordance with this invention can be expressed in vitro and used in a manner in which such immunological and/or expressed products and/or antibodies are typically used, and that cells that express such immunological and/or expressed products and/or antibodies can be employed in in vitro and/or ex vivo applications, e.g., such uses and applications can include diagnostics, assays, ex vivo therapy (e.g., wherein cells that express the gene product and/or immunological response are expanded in vitro and reintroduced into the host or animal), etc., see U.S. Pat. No. 5,990,091, WO 99/60164 and WO 98/00166 and documents cited therein. Further, expressed antibodies or gene products that are isolated from herein methods, or that are isolated from cells expanded in vitro following herein administration methods, can be administered in compositions, akin to the administration of subunit epitopes or antigens or therapeutics or antibodies to induce immunity, stimulate a therapeutic response and/or stimulate passive immunity.

The term “human adenovirus” as used herein is intended to encompass all human adenoviruses of the Adenoviridae family, which include members of the Mastadenovirus genera. To date, over fifty-one human serotypes of adenoviruses have been identified (see, e.g., Fields et al., Virology 2, Ch. 67 (3d ed., Lippincott-Raven Publishers)). The adenovirus can be of serogroup A, B, C, D, E, or F. The human adenovirus can be a serotype 1 (Ad1), serotype 2 (Ad2), serotype 3 (Ad3), serotype 4 (Ad4), serotype 6 (Ad6), serotype 7 (Ad7), serotype 8 (Ad8), serotype 9 (Ad9), serotype 10 (Ad10), serotype 11 (Ad11), serotype 12 (Ad12), serotype 13 (Ad13), serotype 14 (Ad14), serotype 15 (Ad15), serotype 16 (Ad16), serotype 17 (Ad17), serotype 18 (Ad18), serotype 19 (Ad19), serotype 19a (Ad19a), serotype 19p (Ad19p), serotype 20 (Ad20), serotype 21 (Ad21), serotype 22 (Ad22), serotype 23 (Ad23), serotype 24 (Ad24), serotype 25 (Ad25), serotype 26 (Ad26), serotype 27 (Ad27), serotype 28 (Ad28), serotype 29 (Ad29), serotype 30 (Ad30), serotype 31 (Ad31), serotype 32 (Ad32), serotype 33 (Ad33), serotype 34 (Ad34), serotype 35 (Ad35), serotype 36 (Ad36), serotype 37 (Ad37), serotype 38 (Ad38), serotype 39 (Ad39), serotype 40 (Ad40), serotype 41 (Ad41), serotype 42 (Ad42), serotype 43 (Ad43), serotype 44 (Ad44), serotype 45 (Ad45), serotype 46 (Ad46), serotype 47 (Ad47), serotype 48 (Ad48), serotype 49 (Ad49), serotype 50 (Ad50), serotype 51 (Ad51), or serotype 5 (Ad5), but are not limited to these examples.

Also contemplated by the present invention are recombinant vectors, immunogenic compositions, and recombinant adenoviruses that can comprise subviral particles from more than one adenovirus serotype. For example, it is known that adenovirus vectors can display an altered tropism for specific tissues or cell types (Havenga, M. J. E. et al., 2002), and therefore, mixing and matching of different adenoviral capsids, i.e., fiber, or penton proteins from various adenoviral serotypes may be advantageous. Modification of the adenoviral capsids, including fiber and penton can result in an adenoviral vector with a tropism that is different from the unmodified adenovirus. Adenovirus vectors that are modified and optimized in their ability to infect target cells can allow for a significant reduction in the therapeutic or prophylactic dose, resulting in reduced local and disseminated toxicity.

Adenovirus is a non-enveloped DNA virus. Vectors derived from adenoviruses have a number of features that make them particularly useful for gene transfer. As used herein, a “recombinant adenovirus vector” is an adenovirus vector that carries one or more heterologous nucleotide sequences (e.g., two, three, four, five or more heterologous nucleotide sequences). For example, the biology of the adenoviruses is characterized in detail, the adenovirus is not associated with severe human pathology, the virus is extremely efficient in introducing its DNA into the host cell, the virus can infect a wide variety of cells and has a broad host range, the virus can be produced in large quantities with relative ease, and the virus can be rendered replication defective and/or non-replicating by deletions in the early region 1 (“E1”) of the viral genome.

The genome of adenovirus is a linear double-stranded DNA molecule of approximately 36,000 base pairs (“bp”) with a 55-kDa terminal protein covalently bound to the 5′-terminus of each strand. The Ad DNA contains identical Inverted Terminal Repeats (“ITRs”) of about 100 bp, with the exact length depending on the serotype. The viral origins of replication are located within the ITRs exactly at the genome ends. DNA synthesis occurs in two stages. First, replication proceeds by strand displacement, generating a daughter duplex molecule and a parental displaced strand. The displaced strand is single stranded and can form a “panhandle” intermediate, which allows replication initiation and generation of a daughter duplex molecule. Alternatively, replication may proceed from both ends of the genome simultaneously, obviating the requirement to form the panhandle structure.

During the productive infection cycle, the viral genes are expressed in two phases: the early phase, which is the period up to viral DNA replication, and the late phase, which coincides with the initiation of viral DNA replication. During the early phase, only the early gene products, encoded by regions E1, E2, E3 and E4, are expressed, which carry out a number of functions that prepare the cell for synthesis of viral structural proteins (Berk, A. J., 1986). During the late phase, the late viral gene products are expressed in addition to the early gene products and host cell DNA and protein synthesis are shut off. Consequently, the cell becomes dedicated to the production of viral DNA and of viral structural proteins (Tooze, J., 1981).

The E1 region of adenovirus is the first region of adenovirus expressed after infection of the target cell. This region consists of two transcriptional units, the E1A and E1B genes, both of which are required for oncogenic transformation of primary (embryonal) rodent cultures. The main functions of the E1A gene products are to induce quiescent cells to enter the cell cycle and resume cellular DNA synthesis, and to transcriptionally activate the E1B gene and the other early regions (E2, E3 and E4) of the viral genome. Transfection of primary cells with the E1A gene alone can induce unlimited proliferation (immortalization), but does not result in complete transformation. However, expression of E1A, in most cases, results in induction of programmed cell death (apoptosis), and only occasionally is immortalization obtained (Jochemsen et al., 1987). Co-expression of the E1B gene is required to prevent induction of apoptosis and for complete morphological transformation to occur. In established immortal cell lines, high-level expression of E1A can cause complete transformation in the absence of E1B (Roberts, B. E. et al., 1985).

The E1B encoded proteins assist E1A in redirecting the cellular functions to allow viral replication. The E1B 55 kD and E4 33 kD proteins, which form a complex that is essentially localized in the nucleus, function in inhibiting the synthesis of host proteins and in facilitating the expression of viral genes. Their main influence is to establish selective transport of viral mRNAs from the nucleus to the cytoplasm, concomitantly with the onset of the late phase of infection. The E1B 21 kD protein is important for correct temporal control of the productive infection cycle, thereby preventing premature death of the host cell before the virus life cycle has been completed. Mutant viruses incapable of expressing the E1B 21 kD gene product exhibit a shortened infection cycle that is accompanied by excessive degradation of host cell chromosomal DNA (deg-phenotype) and in an enhanced cytopathic effect (cyt-phenotype; Telling et al., 1994). The deg and cyt phenotypes are suppressed when in addition the E1A gene is mutated, indicating that these phenotypes are a function of E1A (White, E. et al., 1988). Furthermore, the E1B 21 kDa protein slows down the rate by which E1A switches on the other viral genes. It is not yet known by which mechanisms E1B 21 kD quenches these E1A dependent functions.

In contrast to, for example, retroviruses, adenoviruses do not integrate into the host cell's genome efficiently, are able to infect non-dividing cells, and are able to efficiently transfer recombinant genes in vivo (Brody et al., 1994). These features make adenoviruses attractive candidates for in vivo gene transfer of, for example, an antigen or immunogen of interest into cells, tissues or subjects in need thereof.

Adenovirus vectors containing multiple deletions may be used to both increase the carrying capacity of the vector and reduce the likelihood of recombination to generate replication competent adenovirus (RCA). Where the adenovirus contains multiple deletions, it is not necessary that each of the deletions, if present alone, would result in a replication defective and/or non-replicating adenovirus. As long as one of the deletions renders the adenovirus replication defective or non-replicating, the additional deletions may be included for other purposes, e.g., to increase the carrying capacity of the adenovirus genome for heterologous nucleotide sequences. In one embodiment, more than one of the deletions prevents the expression of a functional protein and renders the adenovirus replication defective and/or non-replicating and/or attenuated. In another embodiment, all of the deletions are deletions that would render the adenovirus replication-defective and/or non-replicating and/or attenuated. However, the invention also encompasses adenovirus and adenovirus vectors that are replication competent and/or wild-type, i.e. comprises all of the adenoviral genes necessary for infection and replication in a subject.

Embodiments of the invention employing adenovirus recombinants may include E1-defective or deleted, or E3-defective or deleted, or E4-defective or deleted or adenovirus vectors comprising deletions of E1 and E3, or E1 and E4, or E3 and E4, or E1, E3, and E4 deleted, or the “gutless” adenovirus vector in which all viral genes are deleted. The adenovirus vectors can comprise mutations in E1, E3, or E4 genes, or deletions in these or all adenoviral genes. The E1 mutation raises the safety margin of the vector because E1-defective adenovirus mutants are said to be replication-defective and/or non-replicating in non-permissive cells, and are, at the very least, highly attenuated. The E3 mutation enhances the immunogenicity of the antigen by disrupting the mechanism whereby adenovirus down-regulates MHC class 1 molecules. The E4 mutation reduces the immunogenicity of the adenovirus vector by suppressing the late gene expression, thus may allow repeated re-vaccination utilizing the same vector. The present invention comprehends adenovirus vectors of any serotype or serogroup that are deleted or mutated in E1, or E3, or E4, or E1 and E3, or E1 and E4. Deletion or mutation of these adenoviral genes result in impaired or substantially complete loss of activity of these proteins.

The “gutless” adenovirus vector is another type of vector in the adenovirus vector family. Its replication requires a helper virus and a special human 293 cell line expressing both E1a and Cre, a condition that does not exist in natural environment; the vector is deprived of all viral genes, thus the vector as a vaccine carrier is non-immunogenic and may be inoculated multiple times for re-vaccination. The “gutless” adenovirus vector also contains 36 kb space for accommodating antigen or immunogen(s) of interest, thus allowing co-delivery of a large number of antigen or immunogens into cells.

Other adenovirus vector systems known in the art include the AdEasy system (He et al., 1998) and the subsequently modified AdEasier system (Zeng et al., 2001), which were developed to generate recombinant Ad vectors in 293 cells rapidly by allowing homologous recombination between donor vectors and Ad helper vectors to occur in Escherichia coli cells, such as BJ5183 cells, overnight. pAdEasy comprises adenoviral structural sequences that, when supplied in trans with a donor vector such as pShuttle-CMV expressing an antigen or immunogen of interest, results in packaging of the antigen or immunogen (e.g., immunogens and/or antigens) in an adenoviral capsid. The sequence of pAdEasy is well known in the art and is publicly and commercially available through Stratagene.

The present invention can be generated using the AdHigh system (U.S. Patent Provisional Application Ser. No. 60/683,638). AdHigh is a safe, rapid, and efficient method of generating high titers of recombinant adenovirus without the risk of generating RCA, which may be detrimental or fatal to avian subjects. Further, RCA may be pathogenic to humans and undesirable to be present in the food chain. The AdHigh system uses modified shuttle plasmids, such as pAdHigh, to promote the production of RCA-free adenoviruses in permissive cells, such as PER.C6 cells after generating recombinants with an adenovirus backbone plasmid in E. coli cells. These shuttle plasmids contain polylinkers or multiple cloning sites for easy insertion of avian immunogens or antigens such as, for example, avian influenza immunogens or antigens. Recombination of the adenoviral shuttle plasmids in conjunction with an adenoviral helper plasmid such as pAdEasy in bacterial cells (i.e., BJ5183) can be easily implemented to produce the recombinant human adenoviruses expressing avian antigens or immunogens of the invention. Methods of producing recombinant vectors by cloning and restriction analysis are well known to those skilled in the art.

Human Ad5 is not naturally found in birds and the RCA-free Ad5 vector does not replicate in human cells in the absence of E1; thus, the safety profile of immunization regimens using the E1/E3-defective human Ad5 vector is very desirable. Further, such a vector may be used as a vaccine carrier in poultry due to its competence to transduce and incompetence to replicate in avian cells. The Ad5 vector may have transduced a fraction of chicken cells along the mucocutaneous interface through binding of its fiber to the coxsackievirus and adenovirus receptor (CAR) found on the surface of chicken cells (Tan et al., 2001). At least one of the Ad5 components, hexon, is highly immunogenic and confers adjuvant activity to exogenous antigens (Molinier-Frenkel et al., 2002).

Ad5-vectored vaccines mimic the effects of natural infections in their ability to induce major histocompatibility complex (MHC) class I restricted T-cell responses, yet eliminating the possibility of reversion back to virulence because only a subfragment of the pathogen's genome is expressed from the vector. This “selective expression” should be able to solve the problem of differentiating vaccinated-but-uninfected animals from their infected counterparts because the specific markers of the pathogen not encoded by the vector can be used to discriminate the two events. Ad5-vectored vaccines are easily generated, bioengineered, manufactured, and stored. Most notably, propagation of the pathogen is not required for generating vectored vaccines because the relevant antigen genes can be synthesized (Toro et al., 2008). This is particularly important for production of H5N1 AI vaccines because this strain is can be dangerous and difficult to propagate (Wood et al., 2002).

Specific sequence motifs such as the RGD motif may be inserted into the H-I loop of an adenovirus vector to enhance its infectivity. This sequence has been shown to be essential for the interaction of certain extracellular matrix and adhesion proteins with a superfamily of cell-surface receptors called integrins. Insertion of the RGD motif may be advantageously useful in immunocompromised subjects. An adenovirus recombinant is constructed by cloning specific antigen or immunogen or fragments thereof into any of the adenovirus vectors such as those described above. The adenovirus recombinant is used to transduce cells of a vertebrate use as an immunizing agent. (See, for example, U.S. patent application Ser. No. 10/424,409, incorporated by reference).

The adenovirus vectors of the present invention are useful for the delivery of nucleic acids expressing avian antigens or immunogens to cells both in vitro and in vivo. In particular, the inventive vectors may be advantageously employed to deliver or transfer nucleic acids to animal cells. In one embodiment, the animal cells are avian and mammalian cells. Nucleic acids of interest include nucleic acids encoding peptides and proteins. In one embodiment, the nucleic acids may encode therapeutic (e.g., for medical or veterinary uses) or immunogenic (e.g., for vaccines) peptides or proteins.

In one embodiment, the codons encoding the antigen or immunogen of interest are “optimized” codons, i.e., the codons are those that appear frequently in, i.e., highly expressed avian genes, instead of those codons that are frequently used by, for example, influenza.

Such codon usage provides for efficient expression of the antigen or immunogen in human or avian cells. In other embodiments, for example, when the antigen or immunogen of interest is expressed in bacteria, yeast or other expression system, the codon usage pattern is altered to represent the codon bias for highly expressed genes in the organism in which the antigen or immunogen is being expressed. Codon usage patterns are known in the literature for highly expressed genes of many species (e.g., Nakamura et al., 1996; Wang et al, 1998; McEwan et al. 1998).

As a further alternative, the adenovirus vectors can be used to infect a cell in culture to express a desired gene product, e.g., to produce a protein or peptide of interest. In one embodiment, the protein or peptide is secreted into the medium and can be purified therefrom using routine techniques known in the art as well as those provided herein. Signal peptide sequences that direct extracellular secretion of proteins are known in the art and nucleotide sequences encoding the same can be operably linked to the nucleotide sequence encoding the peptide or protein of interest by routine techniques known in the art. Alternatively, the cells can be lysed and the expressed recombinant protein can be purified from the cell lysate. In one embodiment, the cell is an animal cell. In another embodiment, the animal cell is an avian or mammalian cell. In yet another embodiment, the cells may be competent for transduction by adenoviruses.

Such cells include but are not limited to PER.C6 cells, 911 cells, and HEK293 cells. The invention also comprehends the use of avian cells, such as, but not limited to, avian embryonic fibroblasts, such as DF-1 cells, avian stem cells such as those described in U.S. Pat. Nos. 6,872,561; 6,642,042; 6,280,970; and 6,255,108, incorporated by reference, avian lymphoblasts, avian epithelial cells, among others, such as chicken embryo-derived cell strain CHCC-OU2 (Ogura, H. et al., 1987; Japanese Patent Publication No. 9-173059), quail-derived cell strain QT-35 (Japanese Patent Publication No. 9-98778). Any avian cell that is competent for infection, transfection, or any type of gene transfer may be used in the practice of the invention.

A culture medium for culturing host cells includes a medium commonly used for tissue culture, such as M199-earle base, Eagle MEM (E-MEM), Dulbecco MEM (DMEM), SC-UCM102, UP-SFM (GIBCO BRL), EX-CELL302 (Nichirei), EX-CELL293-S (Nichirei), TFBM-01 (Nichirei), ASF104, among others. Suitable culture media for specific cell types can be found at the American Type Culture Collection (ATCC) or the European Collection of Cell Cultures (ECACC). Culture media can be supplemented with amino acids such as L-glutamine, salts, anti-fungal or anti-bacterial agents such as Fungizone®, penicillin-streptomycin, animal serum, and the like. The cell culture medium can optionally be serum-free.

The present invention also provides vectors useful as vaccines. The immunogen or antigen can be presented in the adenovirus capsid, alternatively, the antigen can be expressed from an antigen or immunogen introduced into a recombinant adenovirus genome and carried by the inventive adenoviruses. The adenovirus vector can provide any antigen or immunogen of interest. Examples of immunogens are detailed herein.

The antigens or immunogens may be operably associated with the appropriate expression control sequences. Expression vectors include expression control sequences, such as an origin of replication (which can be bacterial origins, e.g., derived from bacterial vectors such as pBR322, or eukaryotic origins, e.g., autonomously replicating sequences (ARS)), a promoter, an enhancer, and necessary information processing sites, such as ribosome binding sites, RNA splice sites, polyadenylation sites, packaging signals, and transcriptional terminator sequences.

For example, the recombinant adenovirus vectors of the invention can contain appropriate transcription/translation control signals and polyadenylation signals (e.g., polyadenylation signals derived from bovine growth hormone, SV40 polyadenylation signal) operably associated with the antigen or immunogen sequence(s) to be delivered to the target cell. A variety of promoter/enhancer elements may be used depending on the level and tissue-specific expression desired. The promoter can be constitutive or inducible (e.g., the metallothionein promoter), depending on the pattern of expression desired. The promoter may be native or foreign and can be a natural or a synthetic sequence. By foreign, it is intended that the transcriptional initiation region is not found in the wild-type host into which the transcriptional initiation region is introduced. The promoter is chosen so that it will function in the target cell(s) or tissue(s) of interest. Brain-specific, hepatic-specific, and muscle-specific (including skeletal, cardiac, smooth, and/or diaphragm-specific) promoters are contemplated by the present invention. Mammalian and avian promoters are also included in an embodiment of this invention.

The promoter can advantageously be an “early” promoter. An “early” promoter is known in the art and is defined as a promoter that drives expression of a gene that is rapidly and transiently expressed in the absence of de novo protein synthesis. The promoter can also be a “strong” or “weak” promoter. The terms “strong promoter” and “weak promoter” are known in the art and can be defined by the relative frequency of transcription initiation (times per minute) at the promoter. A “strong” or “weak” promoter can also be defined by its affinity to RNA polymerase.

In one embodiment, the antigens or immunogens are operatively associated with, for example, a human cytomegalovirus (CMV) major immediate-early promoter, a simian virus 40 (SV40) promoter, a β-actin promoter, an albumin promoter, an Elongation Factor 1-α (EF1-α) promoter, a PγK promoter, a MFG promoter, or a Rous sarcoma virus promoter. Other expression control sequences include promoters derived from immunoglobin genes, adenovirus, bovine papilloma virus, herpes virus, and so forth. Any mammalian viral promoter can also be used in the practice of the invention, in addition to any avian viral promoter. Among avian promoters of viral origin, the promoters of immediate early (i.e., ICP4, ICP27) genes of the infectious laryngotracheitis virus (ILTV) virus, early (i.e., thymidine kinase, DNA helicase, ribonucleotide reductase) or late (i.e., gB, gD, gC, gK), of the Marek's disease virus, (i.e., gB, gC, pp 38, pp 14, ICP4, Meq) or of the herpes virus of turkeys (i.e., gB, gC, ICP4) can be used in the methods and vectors of the present invention. Moreover, it is well within the purview of the skilled artisan to select a suitable promoter that expresses the antigen or immunogen of interest at sufficiently high levels so as to induce or elicit an immunogenic response to the antigen or immunogen, without undue experimentation.

It has been speculated that driving heterologous nucleotide transcription with the CMV promoter can result in downregulation of expression in immunocompetent animals (see, e.g., Guo et al., 1996). The antigen or immunogen sequences may be operably associated with, for example, a modified CMV promoter that does not result in this down-regulation of antigen or immunogen expression.

The vectors of the invention may also comprise a polylinker or multiple cloning site (“MCS”), which can advantageously be located downstream of a promoter. The polylinker provides a site for insertion of the antigen or immunogen molecules that are “in-frame” with the promoter sequence, resulting in “operably linking” the promoter sequence to the antigen or immunogen of interest. Multiple cloning sites and polylinkers are well known to those skilled in the art. As used herein, the term “operably linked” means that the components described are in a relationship permitting them to function in their intended manner.

Depending on the vector, selectable markers encoding antibiotic resistance may be present when used for in vitro amplification and purification of the recombinant vector, and, in the context of the commercially available AdEasy, AdEasier, and AdHigh adenoviral systems, to monitor homologous recombination between a donor vector and an adenoviral helper vector. The AdEasy, AdEasier, and AdHigh systems facilitate homologous recombination between a donor vector and an adenoviral helper vector at the ITR sequences. Each vector comprises a different antibiotic resistance gene, and by dual selection, recombinants expressing the recombined vector can be selected. Examples of such antibiotic resistance genes that can be incorporated into the vectors of the invention include, but are not limited to, ampicillin, tetracycline, neomycin, zeocin, kanamycin, bleomycin, hygromycin, chloramphenicol, among others.

In embodiments wherein there is more than one antigen or immunogen, the antigen or immunogen sequences may be operatively associated with a single upstream promoter and one or more downstream internal ribosome entry site (IRES) sequences (e.g., the picornavirus EMC IRES sequence).

In embodiments of the invention in which the antigen or immunogen sequence(s) will be transcribed and then translated in the target cells, specific initiation signals are generally required for efficient translation of inserted protein coding sequences. These exogenous translational control sequences, which may include the ATG initiation codon and adjacent sequences, can be of a variety of origins, both natural and synthetic.

Any avian antigen or immunogen derived from an avian pathogen can be used in the methods and recombinant vectors of the invention. In one embodiment, antigens or immunogens include, but are not limited to, antigens or immunogens derived from avian influenza virus, such as hemagglutinin, neuraminidase, matrix, and nucleoprotein antigens or immunogens, infectious bursal disease virus antigens such as VP1, VP1s1, VP1s2, VP2 (Heine, H. G. et al., 1991; Dormitorio, T. V. et al., 1997; and Cao, Y. C. et al., 1998), VP2S, VP3, VP4, VP4S and VP5, Marek's disease virus antigens like thymidine kinase, gA, gB, gC, gD, gE, gH, gI, and gL (Coussens et al. 1988); Ross et al. 1989); Ross et al. 1991); International Publication No. WO 90/02803 (1990); Brunovskis and Velicer, 1995); and Yoshida et al. 1994), Herpesviruses such as infectious laryngotracheitis virus antigens including gA, gB, gD, gE, gI, and gG (Veits, J. et al 2003), avian infectious bronchitis virus antigens such as spike glycoprotein, integral membrane protein M, small membrane protein E, and polyprotein (Casais, R. et al 2003), avian reovirus antigens such as capsid, sigma NS, sigma A, sigma B, and sigma C proteins (Spandidos, D. A. et al, 1976), poxviruses including avipox, fowlpox, canarypox, pigeonpox, quailpox, and dovepox antigens such as thymidine kinase, avian polyomavirus antigens such as VP1, VP2, VP3, and VP4 (Rott, O. et al 1988), Newcastle Disease virus antigens such as HN, P, NP, M, F, and L proteins (reviewed in Alexander, D. J., 1990), avian pneumovirus antigens SH, F, G and N (Seal, B. S., 2000), avian rhinotracheitis virus antigens such as glycoprotein, matrix, fusion, and nucleocapsid (Cook, J. K., 1990), avian reticuloendotheliosis virus antigens such as p29, envelope, gag, protease, and polymerase (Dornburg, R. 1995), avian retroviruses including avian carcinoma virus antigens gag, pol, and env, avian endogenous virus gag, pol, env, capsid, and protease (Rovigatti, U. G. et al, 1983), avian erythroblastosis virus gag, erbA, erbB (Graf, T. et al, 1983), avian hepatitis virus core protein, pol, and surface protein (Cova, L. et al, 1993), avian anemia virus VP1, VP2, VP3 (Rosenberger, J. K. et al, 1998), avian enteritis virus antigens polymerase, 52K protein, penton, Ilia, and core proteins (Pitcovski, J. et al., 1988), Pacheco's disease virus IE protein, glycoprotein K, helicase, glycoprotein N, VP11-12, glycoprotein H, thymidine kinase, glycoprotein B, and nuclear phosphoprotein (Kaleta E. F., 1990), avian leukemia virus antigens envelope, gag, and polymerase (Graf, T. et al, 1978), avian parvovirus, avian rotavirus antigens like NSP1, NSP2, NSP3, NSP4, VP1, VP2, VP3, VP4, VP5, VP6, and VP7 (Mori, Y. et al, 2003; Borgan, M. A. et al, 2003), avian leukosis virus antigens such as envelope, gag, and polymerase (Bieth, E. et al, 1992); avian musculoaponeurotic fibrosarcoma virus (Kawai, S. et al, 1992), avian myeloblastosis virus antigens p15, p27, envelope, gag, and polymerase, nucleocapsid, and gs-b (Joliot, V. et al., 1993), avian myeloblastosis-associated virus (Perbal, B., 1995), avian myelocytomatosis virus (Petropoulos, C. J., 1997), avian sarcoma virus antigens such as p19 and envelope (Neckameyer, W. S. et al, 1985), and avian spleen necrosis virus gag, envelope, and polymerase (Purchase, H. G. et al, 1975).

Other immunogens/antigens that can be used in the context of the present invention include avian bacterial antigens from Pasteurella multocida strains, such as the 39 kDa capsular protein (Ali, H. A. et al, 2004; Rimler, R. B. 2001), 16-kDa outer membrane protein (Kasten, R. W., et al, 1995), lipopolysaccharide (Baert, K. et al, 2005), Escherichia coli, such as type 1 fimbriae, P fimbriae, and curli (Roland, K. et al, 2004); F1 pilus adhesin, P pilus adhesin, aerobactin receptor protein, and lipopolysaccharide (Kariyawasam, S. et al, 2002), Mycoplasma gallisepticum, such as the major membrane antigen pMGA (also known as P67; Jan, G. et al, 2001; Noormohammadi, A. H. et al, 2002a), TM-1 (Saito, S. et al, 1993), adhesin (Barbour, E. K. et al, 1989), P52 (Jan, G. et al, 2001) serum-plate-agglutination (SPA) antigen (Ahmad, I. et al, 1988), Mycoplasma gallinaceum, Mycoplasma gallinarum, Mycoplasma gallopavonis, Mycoplasma synoviae, including antigens such as major membrane antigen MSPB (Noormohammadi, A. H. et al, 2002b) and 165-kDa protein (Ben Abdelmoumen, B. et al, 1999), Mycoplasma meleagridis, Mycoplasma iowae, Mycoplasma pullorum, Mycoplasma imitans, Salmonella enteritidis such as flagellin, porins, OmpA, SEF21 and SEF14 fimbriae (Ochoa-Reparaz, J. et al, 2004), Salmonella enterica serovars such as Gallinarum and Typhimurium that express, for example, SEF14 and SEF21 (Li, W. et al, 2004), Campylobacter jejuni, such as flagellin, 67-kDa antigen, CjaA, CjaC, and CjaD proteins (Widders, P. R. et al, 1998; Wyszynska, A. et al, 2004), Haemophilus paragallinarum such as serogroups A, B, and C antigens like hemagglutinin (Yamaguchi, T. et al, 1988), Riemerella anatipestifer, such as bacterin antigens (Higgins, D. A. et al, 2000), Chlamydia psittaci strains such as serovar A and 6B and expressing, for example, major outer membrane protein (MOMP) (Vanrompay, D. et al, 1999), Erysipelothrix rhusiopathiae including 66-64 kDa protein antigen (Timoney, J. F. et al, 1993), Erysipelothrix insidiosa such as bacterin (Bigland, C. H. and Matsumoto, J. J., 1975), Brucella abortus, such as antigens P39 and bacterioferrin (Al-Mariri, A. et al, 2001), Borrelia anserina such as 22-kilodalton major outer surface protein (Sambri, V. et al, 1999), outer membrane protein P66 (Bunikis, J. et al, 1998), and OspC (Marconi, R. T. et al, 1993), Alcaligenes faecalis, Streptococcus faecalis, Staphylococcus aureus, among many others.

The invention also comprehends the use of immunogens/antigens derived from avian protozoal antigens, such as, but not limited to Eimeria acervulina such as 3-1E antigen (Lillehoj, H. S. et al, 2005; Ding, X. et al, 2004), apical complex antigens (Constantinoiu, C. C. et al, 2004), and lactate dehydrogenase (Schaap, D. et al, 2004), Eimeria maxima such as gam56 and gam82 (Belli, S. I. et al, 2004), 56 and 82 kDa antigen proteins (Belli, S. I. et al, 2002), and EmTFP250 (Witcombe, D. M. et al, 2004), Eimeria necatrix such as 35-kD protein (Tajima, O. et al, 2003), Eimeria tenella such as the TA4 and S07 gene products (Wu, S. Q. et al, 2004; Pogonka, T. et al, 2003) and 12-kDa oocyst wall protein (Karim, M. J. et al, 1996), Eimeria vermiformis, Eimeria adenoeides, Leucocytozoon caulleryi such as R7 outer membrane antigen (Ito, A., et al, 2005), Plasmodium relictum, Plasmodium gallinaceum such as CSP protein (Grim, K. C. et al, 2004) and 17- and 32-kDa protein antigens (Langer, R. C. et al, 2002), and Plasmodium elongatum, among others.

In one embodiment of the invention utilizes avian influenza viral antigens or immunogens. The invention also provides a recombinant vector expressing various avian antigens or immunogens, such as, for example, a multivalent vaccine or immunogenic composition that can protect avians against multiple avian diseases with a single injection.

Avian influenza viruses have been transmitted to humans, pigs, horses, and even sea mammals, and have been key contributors to the emergence of human influenza pandemics. Influenza viruses, which belong to the Orthomyxoviridae family, are classified as A, B, and C based on antigenic differences in their nucleoprotein (NP) and matrix (M1) protein. All avian influenza viruses are classified as type A. Further subtyping is based on the antigenicity of two surface glycoproteins, hemagglutinin (HA) and neuraminidase (NA). Currently, 15 HA and 9 NA subtypes have been identified among influenza A viruses (Murphy, B. R. et al, 1996; Röhm, C. et al, 1996b). The amino acid sequences of the HA1 region, which is responsible for HA antigenicity, differ from subtype to subtype by 30% or more (Röhm, C. et al, 1996b). Although viruses with all HA and NA subtypes are found in avian species, viral subtypes of mammalian influenza viruses are limited.

Avian influenza A viruses are defined by their virulence; highly virulent types that can cause fowl plagues, and avirulent types that generally cause only mild disease or asymptomatic infection. In rare instances, however, viruses with low pathogenicity in the laboratory cause outbreaks of severe disease in the field. Nonetheless, the morbidity and mortality associated with these viruses tend to be much lower than those caused by lethal viruses.

Highly virulent avian influenza viruses have caused outbreaks in poultry in Australia (1976 [H7] (Bashiruddin, J. B. et al, 1992); 1985 [H7] (Cross, G. M., 1987; Nestorowicz, A. et al, 1987), 1992 [H7] (Perdue, M. L. et al, 1997), 1995 [H7], and 1997 [H7]), England (1979 [H7] (Wood, G. W., et al, 1993) and 1991 [H5] (Alexander, D. J. et al, 1993), the United States (1983 to 1984 [H5] (Eckroade, R. J. et al, 1987), Ireland (1983 to 1984 [H5]) (Kawaoka, Y. et al, 1987), Germany (1979 [H7] (Röhm, C. et al, 1996a), Mexico (1994 to 1995 [H5] (Garcia, M. et al, 1996; Horimoto, T. et al, 1995), Pakistan (1995 [H7] (Perdue, M. L. et al, 1997), Italy (1997 [H5]), and Hong Kong (1997 [H5] (Claas, E. J. et al, 1988). Without wishing to be bound by any one theory, it is believed that all of the pathogenic avian influenza A viruses are of the H5 or H7 subtype, although the reason for this subtype specificity remains unknown. There appears to be no association of NA subtypes with virulent viruses. Two additional subtypes, H4 [A/Chicken/Alabama/7395/75 (H4N8)] (Johnson, D. C. et al, 1976) and H10 [A/Chicken/Germany/N/49 (H10N7)], have been isolated from chickens during severe fowl plague-like outbreaks.

The structures of influenza A viruses are quite similar (Lamb, R. A. et al, 1996). By electron microscopy, the viruses are pleomorphic, including virions that are roughly spherical (approximately 120 nm in diameter) and filamentous. Two distinct types of spikes (approximately 16 nm in length), corresponding to the HA and NA molecules, reside on the surface of the virions. These two glycoproteins are anchored to the lipid envelope derived from the plasma membrane of host cells by short sequences of hydrophobic amino acids (transmembrane region). HA is a type I glycoprotein, containing an N-terminal ectodomain and a C-terminal anchor, while NA is a type II glycoprotein, containing an N-proximal anchor and a C-terminal ectodomain. HA enables the virion to attach to cell surface sialyloligosaccharides (Paulson, J. C., 1985) and is responsible for its hemagglutinating activity (Hirst, G. K., 1941). HA elicits virus-neutralizing antibodies that are important in protection against infection. NA is a sialidase (Gottschalk, A., 1957) that prevents virion aggregation by removing cell and virion surface sialic acid (the primary moiety in sialyloligosaccharides recognized by HA) (Paulson, J. C., 1985). Antibodies to NA are also important in protecting hosts (Webster, R. G., et al, 1988).

In addition to HA and NA, a limited number of M1 proteins are integrated into the virions (Zebedee, S. L. et al, 1988). They form tetramers, have H1 ion channel activity, and, when activated by the low pH in endosomes, acidify the inside of the virion, facilitating its uncoating (Pinto, L. H. et al, 1992). M1 protein that lies within the envelope is thought to function in assembly and budding. Eight segments of single-stranded RNA molecules (negative sense, or complementary to mRNA) are contained within the viral envelope, in association with NP and three subunits of viral polymerase (PB1, PB2, and PA), which together form a ribonucleoprotein (RNP) complex that participates in RNA replication and transcription. NS2 protein, now known to exist in virions (Richardson, J. C. et al, 1991; Yasuda, J. et al, 1993), is thought to play a role in the export of RNP from the nucleus (O'Neill, R. E. et al, 1998) through interaction with M1 protein (Ward, A. C. et al, 1995). NS1 protein, the only nonstructural protein of influenza A viruses, has multiple functions, including regulation of splicing and nuclear export of cellular mRNAs as well as stimulation of translation (Lamb, R. A. et al, 1996). Its major function is believed to counteract the interferon activity of the host, since an NS 1 knockout virus was viable although it grew less efficiently than the parent virus in interferon-non-defective cells (Garcia-Sastre, A. et al, 1988).

The avian influenza immunogens or antigens useful in the present invention include, but are not limited to, HA, NA, as well as M1, NS2, and NS1. Particularly avian influenza immunogens or antigens include but are not limited to HA and NA. The avian influenza immunogens or antigens can be derived from any known strain of AI, including all avian influenza A strains, clinical isolates, field isolates, and reassortments thereof. Examples of such strains and subtypes include, but are not limited to, H10N4, H10N5, H10N7, H10N8, H10N9, H11N1, H11N13, H11N2, H11N4, H11N6, H11N8, H11N9, H12N1, H12N4, H12N5, H12N8, H13N2, H13N3, H13N6, H13N7, H14N5, H14N6, H15N8, H15N9, H16N3, H1N1, H1N2, H1N3, H1N6, H2N1, H2N2, H2N3, H2N5, H2N7, H2N8, H2N9, H3N1, H3N2, H3N3, H3N4, H3N5, H3N6, H3N8, H4N1, H4N2, H4N3, H4N4, H4N5, H4N6, H4N8, H4N9, H5N1, H5N2, H5N3, H5N7, H5N8, H5N9, H6N1, H6N2, H6N4, H6N5, H6N6, H6N7, H6N8, H6N9, H7N1, H7N2, H7N3, H7N5, H7N7, H7N8, H8N4, H8N5, H9N1, H9N2, H9N3, H9N5, H9N6, H9N7, H9N8, and H9N9. The invention also relates to the use of mutated or otherwise altered avian influenza immunogens or antigens that reflect, among other things, antigenic drift and antigenic shift.

The antigenicity of influenza viruses changes gradually by point mutation (antigenic drift) or drastically by genetic reassortment (antigenic shift) (Murphy, B. R. et al, 1996). Immunological pressure on HA and NA is thought to drive antigenic drift. Antigenic shift can be caused by either direct transmission of nonhuman influenza viruses to humans or the reassortment of genes from two different influenza viruses that have infected a single cell (Webster, R. G. et al, 1982). Theoretically, 256 different combinations of RNA can be produced from the shuffling of the eight different genomic segments of the virus. Genetic reassortment is well documented both in vitro and in vivo under laboratory conditions (Webster, R. G. et al, 1975). More importantly, mixed infections occur relatively frequently in nature and can lead to genetic reassortment, resulting in new field isolates, hybrid forms, or reassortant forms (Bean, W. J. et al, 1980; Hinshaw, V. S. et al, 1980; Young, J. F., et al, 1979). Reemergence of a previously circulating virus is another mechanism by which antigenic shift can occur.

Thus, the invention also concerns the use of avian influenza immunogens or antigens that have undergone antigenic drift or antigenic shift, including clinical isolates of avian influenza, field or environmental isolates of avian influenza, hybrid forms, and reassortant forms of avian influenza. Moreover, the invention comprehends the use of more than one avian influenza immunogen or antigen in the vectors and methods disclosed herein, delivered either in separate recombinant vectors, or together in one recombinant vector so as to provide a multivalent avian influenza vaccine or immunogenic composition that stimulates or modulates immunogenic response to one or more avian influenza strains and/or hybrids.

Many domestic and wild avian species are infected with influenza viruses. These include chickens, turkeys, ducks, guinea fowl, domestic geese, quail, pheasants, partridge, mynah birds, passerines, psittacines, budgerigars, gulls, shorebirds, seabirds, and emu (Easterday, B. C. et al, 1997; Webster, R. G. et al, 1988). Some infected birds show symptoms of influenza, while others do not. Among domestic avian species, turkeys are the most frequently involved in outbreaks of influenza; chickens have also been involved but less frequently. Avian influenza A viruses produce an array of syndromes in birds, ranging from asymptomatic to mild upper respiratory infections to loss of egg production to rapidly fatal systemic disease (Eckroade, R. J. et al, 1987). The severity of disease depends on multiple factors, including the virulence of the virus, the immune status and diet of the host, accompanying bacterial infections, and stresses imposed on the host. Depending on their pathogenicity in chickens and turkeys, avian influenza A viruses are classified as virulent (capable of causing fowl plague) or avirulent (causing mild or asymptomatic disease). Even when highly pathogenic for one avian species, influenza A viruses may not be pathogenic for another avian species (Alexander, D. J. et al, 1986). For example, ducks are typically resistant to viruses that are lethal in chickens. As another example, A/Turkey/Ireland/1378/85 (H5N8), which readily kills chickens and turkeys, does not cause disease symptoms in ducks, even though it can be detected in a variety of internal organs and in the blood of infected birds (Kawaoka, Y. et al, 1987).

Influenza viruses are secreted from the intestinal tract into the feces of infected birds (Kida, H., et al, 1980; Webster, R. G. et al, 1978). The modes of transmission can be either direct or indirect; they include contact with aerosol and other virus-contaminated materials. Since infected birds excrete large amounts of virus in their feces, many different items can become contaminated (e.g., feed, water, equipment, and cages) and contribute to dissemination of the virus. Waterborne transmission may provide a mechanism for the year-to-year perpetuation of avian influenza viruses in natural waterfowl habitats. The typical signs and symptoms manifested by commercial avians, such as poultry infected with highly pathogenic avian influenza viruses include decreased egg production, respiratory signs, rales, excessive lacrimation, sinusitis, cyanosis of unfeathered skin (especially the combs and wattles), edema of the head and face, ruffled feathers, diarrhea, and nervous system disorders.

The number of presenting features depends on the species and age of the bird, the strain of virus, and accompanying bacterial infections (Easterday, B. C. et al., 1997; Webster, R. G. et al., 1988). Occasionally, a bird will die without showing any signs of illness (Alexander, D. J. et al., 1993; Wood, G. W., et al., 1994). The gross and histological lesions in chickens inoculated with highly pathogenic viruses are quite similar but do show some strain variation (Alexander, D. J. et al., 1986; Mo, I. P. et al., 1997; Swayne, D. E. et al., 1997). Some of the differences among reported cases may reflect differences in experimental conditions, including the route of inoculation, the breed and age of the chickens, and the dose of virus. Swelling of the microvascular endothelium, systemic congestion, multifocal hemorrhages, perivascular mononuclear cell infiltration, and thrombosis are commonly seen in chickens infected with highly virulent viruses. Such viruses replicate efficiently in the vascular endothelium and perivascular parenchymatous cells, a property that can be important for viral dissemination and systemic infection (Kobayashi, Y. et al, 1996; Suarez, D. L. et al, 1998). Viral antigens can also be found in necrotic cardiac myocytes in addition to cells in other organs with necrotic and inflammatory changes (Kobayashi, Y. et al., 1996).

The present invention also relates to methods of expressing one or more antigens or immunogens in a cell. As a preliminary step in the laboratory setting, the antigen or immunogen can instead be replaced by a heterologous nucleotide sequences encoding proteins and peptides that include those encoding reporter proteins (e.g., an enzyme). Reporter proteins are known in the art and include, but are not limited to, Green Fluorescent Protein, β-galactosidase, β-glucuronidase, luciferase, alkaline phosphatase, and chloramphenicol acetyltransferase gene. Many of these reporter proteins and methods of their detection are included as a part of many commercially available diagnostic kits. The antigen or immunogen of interest may encode an antisense nucleic acid, small interfering RNAs (siRNAs), a ribozyme, or other non-translated RNAs, such as “guide” RNAs (Gorman et al., 1998), and the like.

The recombinant vectors and methods of the invention also comprehend the use of therapeutic proteins or adjuvant molecules that can modulate immune responses upon delivery of recombinant vectors or immunogenic compositions. Such therapeutic proteins or adjuvant molecules can include, but are not limited to, immunomodulatory molecules such as interleukins, interferon, and co-stimulatory molecules. Avian cytokines that are known in the art to modulate immune responses in an avian subject are chicken interferon-α (IFNα) (Karaca, K. et al., 1998; Schijns, V. E. et al., 2000), chicken interferon-γ (IFNγ), chicken interleukin-1β (ChIL-1β) (Karaca, K. et al., 1998), chicken interleukin-2 (ChIL-2) (Hilton, L. S. et al., 2002), and chicken myelo-monocytic growth factor (cMGF1 York, J. J. et al., 1996; Djeraba, A. et al., 2002). The immunomodulatory molecules can be co-administered with the inventive immunogenic compositions, or alternatively, the nucleic acid of the immunomodulatory molecule(s) can be co-expressed along with the avian immunogens or antigens in the recombinant vectors of the invention.

Expression in the subject of the heterologous sequence, i.e. avian influenza immunogens, can result in an immune response in the subject to the expression products of the antigen or immunogen. Thus, the recombinant vectors of the present invention may be used in an immunological composition or vaccine to provide a means to induce an immune response, which may be, but need not be, protective. The molecular biology techniques used in the context of the invention are described by Sambrook et al. (2001).

Even further alternatively or additionally, in the immunogenic or immunological compositions encompassed by the present invention, the nucleotide sequence encoding the antigens or immunogens can have deleted therefrom a portion encoding a transmembrane domain. Yet even further alternatively or additionally, the vector or immunogenic composition can further contain and express in a host cell a nucleotide sequence encoding a heterologous tPA signal sequence such as human or avian tPA and/or a stabilizing intron, such as intron II of the rabbit β-globin gene.

The present invention also provides a method of delivering and/or administering a heterologous nucleotide sequence into a cell in vitro or in vivo. According to this method a cell is infected with a recombinant human adenovirus vector according to the present invention (as described in detail herein). The cell may be infected with the adenovirus vector by the natural process of viral transduction. Alternatively, the vector may be introduced into the cell by any other method known in the art. For example, the cell may be contacted with a targeted adenovirus vector (as described below) and taken up by an alternate mechanism, e.g., by receptor-mediated endocytosis. As another example the vector may be targeted to an internalizing cell-surface protein using an antibody or other binding protein.

A vector can be administered to an avian subject in an amount to achieve the amounts stated for gene product (e.g., epitope, antigen, therapeutic, and/or antibody) compositions. The invention includes but is not limited to dosages below and above those exemplified herein, and for any composition to be administered to an avian subject, including the components thereof, and for any particular method of administration, the following may be determined: toxicity, such as by determining the lethal dose (LD) and LD₅₀ in a suitable avian model; and, the dosage of the composition(s), concentration of components therein and timing of administering the composition(s), which elicit a suitable response, such as by titrations of sera and analysis thereof, e.g., by ELISA and/or seroneutralization analysis. Such determinations do not require undue experimentation from the knowledge of the skilled artisan, this disclosure and the documents cited herein.

Examples of compositions of the invention include but are not limited to liquid preparations for orifice, or mucosal, e.g., oral, nasal, anal, vaginal, peroral, intragastric, etc., administration such as suspensions, solutions, sprays, syrups or elixirs; and, preparations for parenteral, epicutaneous, subcutaneous (i.e., through lower neck), intradermal, intraperitoneal, intramuscular (i.e., through wing-web, wing-tip, pectoral, and thigh musculature puncture), intranasal, or intravenous administration (e.g., injectable administration) such as sterile suspensions or emulsions. Reference is made to U.S. Pat. No. 6,716,823 issued Apr. 6, 2004; U.S. Pat. No. 6,706,693 issued Mar. 16, 2004; U.S. Pat. No. 6,348,450 issued Feb. 19, 2002; U.S. application Ser. Nos. 10/052,323 and 10/116,963; and 10/346,021, the contents of which are incorporated herein by reference and which disclose immunization and delivery of immunogenic or vaccine compositions through a non-invasive mode of delivery, i.e. epicutaneous and intranasal administration. Other methods of administration and delivery to avians include administering the recombinant vectors or immunogenic compositions in drinking water or feed, wherein the dose of vaccine can be selected between 10¹ and 10⁴ per animal.

For intramuscular injections, administration can occur through the breast (pectoral), leg (upper thigh/lateral flank musculature), wing-web (patagium), under wings (axilla), and wing-tip. The length and diameter (gauge) of the needle used should be such that it will allow delivery of the vaccine to the center of the chosen muscle.

A particular embodiment includes a method of administration such as in ovo delivery (Gildersleeve, R. P., 1993a; Gildersleeve, R. P. et al, 1993b; Sharma, J. M., 1985; Sharma, J. M. et al, 1984). In ovo delivery is emerging as a promising method for mass immunization of avians as administration of a uniform dose of vaccines by a robotic injector is both labor- and time-saving (Johnston et al., 1997; Oshop et al., 2002). To date, over 80% of U.S. commercial broiler chickens are treated in ovo with a mechanized injector against Marek's disease (Wakenell et al., 2002). This method is also being used increasingly to administer infectious bursal disease (IBD) and Newcastle disease (ND) vaccines. Immune responses have also been elicited in chickens by in ovo delivery of DNA vaccines (Kapczynski et al., 2003; Oshop et al., 2003) and a replicating alphavirus-vectored vaccine (Schultz-Cherry et al., 2000). Compared with their replicating counterparts, DNA and viral vectored in ovo vaccines and immunogenic compositions are less likely to kill or harm the embryo and Ad vectors in particular have a higher compliance rate due to their incompetence to replicate in ovo.

Mechanized systems, apparatuses, and devices, such as those commercially available as INOVOJECT®, gently inject compounds, vaccines, and immunogenic compositions in precisely calibrated volumes without causing trauma to the developing embryo, thereby reducing chick handling, improving hatchery manageability through automation, and reducing costs of live production. INOVOJECT® and other mechanized systems, devices, or apparatuses, work by gently lowering an injection head onto the top of the egg and a small diameter hollow punch pierces a small opening in the shell. A needle descends through a tube to a controlled depth (usually 2.54 cm), a small, pre-determined volume of vaccine, immunogenic composition, or compound is delivered to the embryo, and then the needle is withdrawn and cleansed in a sterilization wash. Methods of in ovo vaccine and gene delivery can be found in U.S. Pat. Nos. 4,458,630; RE 35973; 6,668,753; 6,601,534; 6,506,385; 6,395,961; 6,286,455; 6,244,214; 6,240,877; 6,032,612; 5,784,992; 5,699,751; 5,438,954; 5,339,766; 5,176,101; 5,136,979; 5,056,464; 4,903,635; 4,681,063; U.S. application Ser. Nos. 10/686,762, filed on Oct. 16, 2003; 10/216,427, filed on Aug. 9, 2002; 10/074/714, filed on Feb. 13, 2002; and 10/043,025, filed on Jan. 9, 2002, the contents of which are expressly incorporated by reference. Accordingly, the invention contemplates a device or apparatus for in ovo delivery or administration of the recombinant vectors, vaccine or immunogenic compositions described herein. The device or apparatus can optionally comprise the recombinant human adenovirus vectors or immunogenic compositions of the present invention, i.e. can be pre-loaded with the vectors or immunogenic compositions for in ovo administration into an avian.

A further method of administration is aerosol spray delivery. It has been shown that intranasal administration of human Ad5-vectored vaccines failed to immunize chickens (Gao et al., 2006; Toro et al., 2007). The surprising demonstration herein that aerosol spray of human Ad5-vectored vaccines was effective in vaccinating chickens may be attributed to intraocular administration, a combination of multiple routes (intranasal, intraocular, transdermal, and oral administration), and/or the fine mist generated during aerosol spray.

Aerosol spray delivery is a useful method for mass immunization of avians that is both labor- and time-saving, such that commercial concerns heavily favor method of administration such as aerosol spray in the poultry industry.

Mechanized systems, apparatuses, and devices that allow such aerosol spray delivery are therefore encompassed by the present invention. Accordingly, the invention contemplates a device or apparatus for aerosol spray delivery or administration of the recombinant vectors, vaccine or immunogenic compositions described herein. The device or apparatus can optionally comprise the recombinant human adenovirus vectors or immunogenic compositions of the present invention, i.e. can be pre-loaded with the vectors or immunogenic compositions for aerosol spray administration into an avian.

The invention also comprehends sequential administration of inventive compositions or sequential performance of herein methods, e.g., periodic administration of inventive compositions such as in the course of therapy or treatment for a condition and/or booster administration of immunological compositions and/or in prime-boost regimens; and, the time and manner for sequential administrations can be ascertained without undue experimentation.

Further, the invention comprehends compositions and methods for making and using vectors, including methods for producing gene products and/or immunological products and/or antibodies in vivo and/or in vitro and/or ex vivo (e.g., the latter two being, for instance, after isolation therefrom from cells from a host that has had an administration according to the invention, e.g., after optional expansion of such cells), and uses for such gene and/or immunological products and/or antibodies, including in diagnostics, assays, therapies, treatments, and the like.

Vector compositions are formulated by admixing the vector with a suitable carrier or diluent; and, gene product and/or immunological product and/or antibody compositions are likewise formulated by admixing the gene and/or immunological product and/or antibody with a suitable carrier or diluent; see, e.g., U.S. Pat. No. 5,990,091, WO 99/60164, WO 98/00166, documents cited therein, and other documents cited herein, and other teachings herein (for instance, with respect to carriers, diluents and the like).

In such compositions, the recombinant vectors may be in admixture with a suitable veterinarily or pharmaceutically acceptable carrier, diluent, or excipient such as sterile water, physiological saline, glucose or the like. The compositions can also be lyophilized. The compositions can contain auxiliary substances, such as wetting or emulsifying agents, pH buffering agents, adjuvants, gelling or viscosity enhancing additives, preservatives, flavoring agents, colors, and the like, depending upon the route of administration and the preparation desired.

DMSO has been known to enhance the potency of vaccine and immunogenic compositions, particularly in regard to in ovo or aerosol spray delivery of vectors or immunogenic compositions comprising vectors. DMSO is thought to enhance the potency of vaccines by increasing the permeability of cellular membranes (Oshop et al, 2003). Other agents or additives that are capable of permeabilizing cells, reducing the viscosity of amniotic fluid, and exhibiting a higher compliance rate as compared to DMSO can be used in the formulation of vaccines or immunogenic compositions, especially when administered by in ovo or aerosol spray delivery. Absorption of a variety of proteins, such as insulin, leptin, and somatotropin, have been shown to be enhanced by surfactants such as tetradecyl maltoside (TDM) without appreciable side effects, following intranasal administration (Arnold, et al, 2004). The present invention therefore comprehends the use of TDM in the methods and compositions described herein.

Formulations containing 0.125% TDM can cause moderate alterations in cell morphology, while higher concentrations of TDM (i.e., 0.5%) can transiently induce more extensive morphological changes. TDM is believed to enhance vector delivery in an in ovo or aerosol spray delivery setting due to the viscous nasal mucus in mammals and amniotic fluid of embryonated avian eggs. The safety profile of TDM as described in Arnold, et al (2004) is also particularly advantageous to promote the health of immunized avians and compliance for entering the food chain.

The quantity of vector to be administered will vary for the subject and condition being treated and will vary from one or a few to a few hundred or thousand micrograms of body weight per day and the dose of vaccine or immunogenic composition being chosen preferably between 10¹-10⁶ plaque forming units (PFU), preferably 10²-10⁵ PFU per bird. For injection, vaccines containing the above titer should be diluted with a pharmaceutically or veterinarily acceptable liquid such as physiological saline to a final volume of approximately 0.1 ml or 0.01 ml in the case of wing web administration. The vectors and methods of the present invention permit vaccination in ovo and vaccination by aerosol spray of 1-day-old chicks, as well as vaccination of older chicks and adults.

A vector can be non-invasively administered to an avian subject in an amount to achieve the amounts stated for gene product (e.g., epitope, antigen, therapeutic, and/or antibody) compositions. Of course, the invention envisages dosages below and above those exemplified herein, and for any composition to be administered to an avian subject, including the components thereof, and for any particular method of administration, the following may therefore be determine: toxicity, such as by determining the lethal dose (LD) and LD₅₀ in a suitable avian model; and, the dosage of the composition(s), concentration of components therein and timing of administering the composition(s), which elicit a suitable response, such as by titrations of sera and analysis thereof, e.g., by ELISA and/or seroneutralization analysis. Such determinations do not require undue experimentation from the knowledge of the skilled artisan, this disclosure and the documents cited herein.

Recombinant vectors can be administered in a suitable amount to obtain in vivo expression corresponding to the dosages described herein and/or in herein cited documents. For instance, suitable ranges for viral suspensions can be determined empirically. If more than one gene product is expressed by more than one recombinant, each recombinant can be administered in these amounts; or, each recombinant can be administered such that there is, in combination, a sum of recombinants comprising these amounts.

In vector or plasmid compositions employed in the invention, dosages can be as described in documents cited herein or as described herein or as in documents referenced or cited in herein cited documents. Advantageously, the dosage should be a sufficient amount of plasmid to elicit a response analogous to compositions wherein the antigen(s) are directly present; or to have expression analogous to dosages in such compositions; or to have expression analogous to expression obtained in vivo by recombinant compositions.

However, the dosage of the composition(s), concentration of components therein and timing of administering the composition(s), which elicit a suitable immunological response, can be determined by methods such as by antibody titrations of sera, e.g., by ELISA and/or seroneutralization assay analysis. Such determinations do not require undue experimentation from the knowledge of the skilled artisan, this disclosure and the documents cited herein. And, the time for sequential administrations can be likewise ascertained with methods ascertainable from this disclosure, and the knowledge in the art, without undue experimentation.

The immunogenic or immunological compositions contemplated by the invention can also contain an adjuvant. Suitable adjuvants include fMLP(N-formyl-methionyl-leucyl-phenylalanine; U.S. Pat. No. 6,017,537) and/or acrylic acid or methacrylic acid polymer and/or a copolymer of maleic anhydride and of alkenyl derivative. The acrylic acid or methacrylic acid polymers can be cross-linked, e.g., with polyalkenyl ethers of sugars or of polyalcohols. These compounds are known under the term “carbomer” (Pharmeuropa, Vol. 8, No. 2, June 1996). A person skilled in the art may also refer to U.S. Pat. No. 2,909,462 (incorporated by reference), which discusses such acrylic polymers cross-linked with a polyhydroxylated compound containing at least 3 hydroxyl groups: in one embodiment, a polyhydroxylated compound contains not more than 8 hydroxyl groups; in another embodiment, the hydrogen atoms of at least 3 hydroxyls are replaced with unsaturated aliphatic radicals containing at least 2 carbon atoms; in other embodiments, radicals contain from about 2 to about 4 carbon atoms, e.g., vinyls, allyls and other ethylenically unsaturated groups. The unsaturated radicals can themselves contain other substituents, such as methyl. The products sold under the name Carbopol® (Noveon Inc., Ohio, USA) are particularly suitable for use as an adjuvant. They are cross-linked with an allyl sucrose or with allylpentaerythritol, as to which, mention is made of the products Carbopol® 974P, 934P, and 971P.

As to the copolymers of maleic anhydride and of alkenyl derivative, mention is made of the EMA® products (Monsanto), which are copolymers of maleic anhydride and of ethylene, which may be linear or cross-linked, for example cross-linked with divinyl ether. Also, reference may be made to U.S. Pat. No. 6,713,068 and Regelson, W. et al., 1960; incorporated by reference).

Cationic lipids containing a quaternary ammonium salt are described in U.S. Pat. No. 6,713,068, the contents of which are incorporated by reference, can also be used in the methods and compositions of the present invention. These cationic lipids include but are not limited to DMRIE (N-(2-hydroxyethyl)-N,N-dimethyl-2,3-bis(tetradecyloxy)-1-propane ammonium; WO96/34109), advantageously associated with a neutral lipid, advantageously DOPE (dioleoyl-phosphatidyl-ethanol amine; Behr J. P., 1994), to form DMRIE-DOPE.

A recombinant vaccine or immunogenic or immunological composition can also be formulated in the form of an oil-in-water emulsion. The oil-in-water emulsion can be based, for example, on light liquid paraffin oil (European Pharmacopea type); isoprenoid oil such as squalane, squalene, EICOSANE™ or tetratetracontane; oil resulting from the oligomerization of alkene(s), e.g., isobutene or decene; esters of acids or of alcohols containing a linear alkyl group, such as plant oils, ethyl oleate, propylene glycol di(caprylate/caprate), glyceryl tri(caprylate/caprate) or propylene glycol dioleate; esters of branched fatty acids or alcohols, e.g., isostearic acid esters. The oil advantageously is used in combination with emulsifiers to form the emulsion. The emulsifiers can be nonionic surfactants, such as esters of sorbitan, mannide (e.g., anhydromannitol oleate), glycerol, polyglycerol, propylene glycol, and oleic, isostearic, ricinoleic, or hydroxystearic acid, which are optionally ethoxylated, and polyoxypropylene-polyoxyethylene copolymer blocks, such as the Pluronic® products, e.g., L121. The adjuvant can be a mixture of emulsifier(s), micelle-forming agent, and oil such as that which is available under the name Provax® (IDEC Pharmaceuticals, San Diego, Calif.).

The recombinant adenovirus, or recombinant adenoviral vector expressing one or more antigen or immunogen of interest, e.g., vector according to this disclosure, can be preserved and/or conserved and stored either in liquid form, at about 5° C., or in lyophilized or freeze-dried form, in the presence of a stabilizer. Freeze-drying can be according to well-known standard freeze-drying procedures. The pharmaceutically acceptable stabilizers may be SPGA (sucrose phosphate glutamate albumin; Bovarnick, et al., 1950), carbohydrates (e.g., sorbitol, mannitol, lactose, sucrose, glucose, dextran, trehalose), sodium glutamate (Tsvetkov, T. et al., 1983; Israeli, E. et al., 1993), proteins such as peptone, albumin or casein, protein containing agents such as skimmed milk (Mills, C. K. et al., 1988; Wolff, E. et al., 1990), and buffers (e.g., phosphate buffer, alkaline metal phosphate buffer). An adjuvant and/or a vehicle or excipient may be used to make soluble the freeze-dried preparations.

The invention will now be further described by way of the following non-limiting Examples, given by way of illustration of various embodiments of the invention and are not meant to limit the present invention in any fashion.

### EXAMPLES

### Example 1

### Construction of an Ad Vector Encoding the A/Panama/2007/99 HA

The influenza virus strain, A/Panama/2007/99 (H3N2) (SEQ ID NOs: 1, 2), selected for vaccine production in 2003-2004, was provided by the Centers for Disease Control (CDC). The hemagglutinin (HA) gene was cloned by reverse transcription of the influenza RNA, followed by amplification of the HA gene with polymerase chain reaction (PCR) using the following primers in Table 1.

TABLE 1
Primers Used in Construction of Ad vectors

| Primer | Sequence |
|---|---|
| 5′ HA | 5′-CACACAGGTACCGCCATGAAGACTATCAT |
| | TGCTTTGAGC-3′ (SEQ ID NO: 9) |
| 3′ HA | 5′-CACACAGGTACCTCAAATGCAAATGTTGCACC-3′ |
| | (SEQ ID NO: 10) |

These primers contain sequences that anneal to the 5′ and 3′ ends of the A/Panama/2007/99 HA gene, as well as sequences corresponding to an eukaryotic ribosomal binding site (Kozak, 1986) immediately upstream from the HA initiation ATG codon, and KpnI sites for subsequent cloning. The KpnI fragment containing the full-length HA gene was inserted into the KpnI site of pShuttle-CMV (He et al., 1998) (provided by T. He) in the correct orientation under transcriptional control of the human cytomegalovirus (CMV) early promoter. An E1/E3-defective Ad5 vector encoding the A/Panama/2007/99 HA (AdPNM2007/99.H3) was generated in human 293 cells using a simplified recombination system as described (Zeng et al., 2001).

The AdPNM2007/99.H3 vector was validated by sequencing both 5′ and 3′ junctions between the HA insert and vector. HI antibodies against A/Panama/2007/99 were elicited in mice after intranasal administration of AdPNM2007/99.H3.

### Example 2

### Immunization of Chickens by in Ovo and Intramuscular Injection of a Recombinant Ad Vector

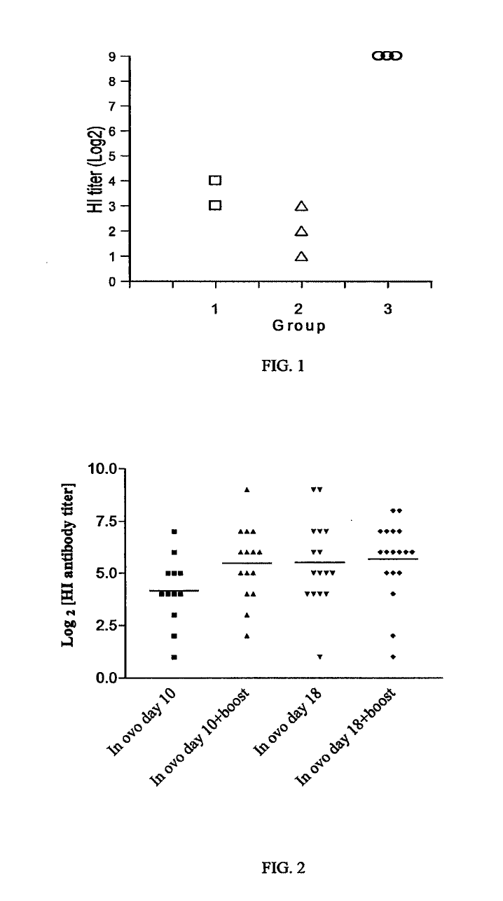

Immunization of chickens by inoculation of a human Ad-vectored vaccine has not been heretofore reported. Since Ad5 is not naturally found in birds, this vector was believed to be unable to infect and/or replicate in chicken cells efficiently. Surprisingly, serum HI titers of 512 were achieved in all 3 chickens two weeks after intramuscular injection of AdPNM2007/99.H3 (FIG. 1).

When AdPNM2007/99.H3 vectors were injected into 9-day-old and 18-day-old embryonated chicken eggs, serum HI titers of 8 and 16 were achieved in the former, and HI titers of <4, 4, and 8 were achieved in the latter two weeks post-hatch. The results suggest that the E1/E3-defective human Ad5 vector can be used as a vaccine carrier in avians due to its competence to transduce and inability to replicate in avian cells. The relatively low HI titers induced by in ovo vaccination may be attributed to, among other things, the dosage and the age of the embryos. The Ad5 vector may have transduced part of the chicken embryo through binding of its fiber to the coxsackievirus and adenovirus receptor (CAR) found on the surface of chicken cells (Tan et al., 2001). An immune response can be elicited in chickens following transduction of only a small number of cells, because Ad is a potent vector capable of protecting the vector DNA by disrupting endosomes after internalization (Curiel, 1994). In addition, at least one of the Ad components, the hexon, is highly immunogenic and confers adjuvant activity to exogenous antigens (Molinier-Frenkel et al., 02).

The AdPNM2007/99.H3 vector was injected into the amnion of 9-day-old (Group 1) and 18-day-old (Group 2) embryonated chicken eggs, respectively, in a volume of 200 μl at a dose of 5×10¹⁰ pfu per egg. There were 6 eggs per group; however, only 2 birds hatched in Group 1 and 3 birds in Group 2. Serum HI titers were determined as described (Van Kampen et al., 2005) 2 weeks post-hatch. In Group 3, the AdPNM2007/99.H3 vector was injected intramuscularly into three 4-week-old chickens in a volume of 100 μl at a dose of 2.5×10¹⁰ pfu per animal. HI titers were determined two weeks post-immunization.

In Group 1 (in ovo immunization of 9-day-old embryos), HI titers of 8 and 16 were achieved; in Group 2 (in ovo immunization of 18-day-old embryos), HI titers of <4 (arbitrarily assigned a titer of 2), 4, and 8 were achieved; and in Group 3 (intramuscular immunization of 4-week-old chickens), HI titers of 512 were achieved in all three birds. FIG. 1 shows the HI titers on log₂ scale. The squares correspond to HI titers in individual birds in Group 1; while the triangles correlate to HI titers in individual birds in Group 2. The circles correspond to HI titers in individual birds in Group 3.

### Example 3

### Construction of an Ad Vector Encoding the A/turkey/Wisconsin/68 H Gene (AdTW68. H5)

The DNA template of the A/Turkey/WI/68 H (SEQ ID NOs: 3, 4) encoding the H of the AI virus strain, was provided by USDA Southeast Poultry Research Laboratory, Athens Ga., and was PCR amplified using the primers shown in Table 2.

TABLE 2
Primers Used in Construction of Ad vectors

| Primer | Sequence |
|---|---|
| 5′ HA | 5′CACACAAAGCTTGCCGCCATGGAAAGAATAGTGATTGC3′ |
| | (SEQ ID NO: 10) |
| 3′ HA | 5′CACACAGGATCCATCTGAACTCACAATCCTAGATGC3′ |
| | (SEQ ID NO: 11) |

These primers contain sequences that anneal to the 5′ and 3′ ends of the A/turkey/Wisconsin/68 H gene, an eukaryotic ribosomal binding site (Kozak, 1986) immediately upstream from the H initiation ATG codon, and unique restriction sites for subsequent cloning. The fragment containing the full-length H gene was inserted into the HindIII-BamHI site of the shuttle plasmid pAdApt (provided by Crucell, Leiden, The Netherlands) in the correct orientation under transcriptional control of the human cytomegalovirus (CMV) early promoter. An RCA-free, E1/E3-defective Ad vector encoding the A/turkey/Wisconsin/68 H gene (AdTW68.H5) was subsequently generated in human PER.C6 cells (provided by Crucell) by co-transfection of pAdApt-TW68.H5 with the Ad5 backbone plasmid pAdEasy1 (He et al., 1998) as described (Shi et al., 2001). The AdTW68.H5 vector was validated by sequencing both 5′ and 3′ junctions between the H insert and the vector backbone.

Ad-vectored in ovo AI vaccines may be produced rapidly and mass-administered into chicken populations within the context of a superb safety profile in response to an emerging AI pandemic. Large-scale production of RCA-free Ad5 vectors in the well-characterized PER.C6 cell line in serum-free suspension bioreactors (Lewis, 2006) in conjunction with chromatography-mediated purification (Konz, 2005) and buffers that do not require freezers for long-term storage (Evans, 2004) should greatly reduce the production costs of Ad5 vectors. The use of cultured cells instead of embryonated eggs as a substrate for AI vaccine production is significant, particularly during an AI outbreak when fertile eggs may be in short supply. This Ad5-vectored AI vaccine is in compliance with a DIVA strategy because the vector only encodes the viral HA. Thus, analysis of serum HI antibodies together with measurement of anti-AI nucleoprotein by enzyme-linked immunosorbent assay would allow rapid determination of exposure to the AI vaccine or virus.

The RCA-free Ad5-vectored in ovo AI vaccine provides a unique platform capable of arresting HPAI virus infections in immunized birds through automated delivery of a uniform dose of non-replicating AI vaccine that is compatible with a DIVA strategy. Unlike replicating recombinant vectors that are associated with the risk of generating revertants and allow spread of genetically modified organisms in both target and non-target species in the environment, the RCA-free Ad5 vectors will not propagate in the field. In contrast to the reassortant AI virus vaccine that may generate undesirable further reassortments with a concurrently circulating wild influenza virus (Hilleman, 2002), it is not possible for the DNA genome of Ad5 to undergo reassortment with the segmented RNA genome of an influenza virus.

### Example 4

### In Ovo Inoculation of AdTW68.H5

In ovo inoculation was performed as described (Senne, 1998; Sharma et al., 1982). Before inoculation all embryos were candled for viability, and the site of inoculation marked and disinfected with a solution of 70% ethyl alcohol containing 3.5% iodine. A hole was made in the shell using a rotating drill equipped with a pointed tip. Inoculation was performed by the amnion-allantoic route by use of 1 ml syringes. After inoculation, the hole was sealed with melted paraffin.

### Example 5

### Serology Post-Inoculation of AdTW68.H5

AI strain A/turkey/WI/68 was passaged in SPF embryonated chicken eggs to achieve a titer of 10⁶ embryo infective doses 50%/ml. Amnioallantoic fluids were tested for hemagglutinating activity. Antibody titers in individual serum samples were determined by hemagglutination inhibition using 4 hemagglutinating units of the AI virus as described (Swayne et al., 1998, Thayer et al., 1998).

### Example 6

### Sampling and Quantification of AI Genomes

Oro-pharyngeal samples from individual birds were suspended in 1.0 ml of brain heart infusion medium (Difco, Detroit, Mich.) and stored at −70° C. RNA was extracted by using the RNeasy mini kit (Qiagen). Quantitative real-time RT-PCR was performed with primers specific for type A influenza virus matrix RNA, as described previously (Spackman, 2002). Viral RNA was interpolated from the cycle thresholds by using standard curves generated from known amounts of control A/chicken/Queretaro/14588-19/95 RNA (10^{1.0} to 10^{6.0} EID₅₀/mL).

### Example 7

### Immunization of Chickens by in Ovo Inoculation of AdTW68.H5 Followed by Post-Hatch Boost

Immunization of chickens by inoculation was accomplished by administering 300 μl of the AdTW68.H5 containing 10¹¹ viral particles/ml (vp/ml) into SPF embryonated eggs on days 10 or 18 of embryonation. Hatched chicks of each group were equally divided into two groups: half of the chickens were revaccinated via the nasal route with the same dose of AdTW68.H5 at day 15 post-hatch, and the remaining chickens did not receive a booster application post-hatch.

The HI antibody titers detected in sera obtained at day 28 post-hatch from these bird groups are shown in FIG. 2. Chicks vaccinated in ovo on day 10 of embryonation showed HI titers varying between 2 and 7 log₂ (mean of 4.2); chickens vaccinated at day 10 of embryonation with post-hatch booster application showed HI titers varying between 2 and 9 log₂ (mean of 5.5); chicks vaccinated at day 18 of embryonation showed titers varying between 2 and 9 log₂ (mean of 5.5); and chickens vaccinated at day 18 of embryonation and boosted at day 15 post-hatch showed HI values between 2 and 8 log₂ (mean of 5.7). Overall, in ovo administration of this human Ad-vectored AI vaccine induced a robust immune response against AI in chickens, whereas intranasal instillation of this vectored vaccine into chickens, as recently demonstrated (Gao, 2006), is ineffective.

### Example 8

### In Ovo Inoculation of AdTW68.H5 Protects Against Lethal Challenge with the Highly Pathogenic Avian Influenza Strain HPAI A/chicken/Queretaro/19/95 (H5N2)

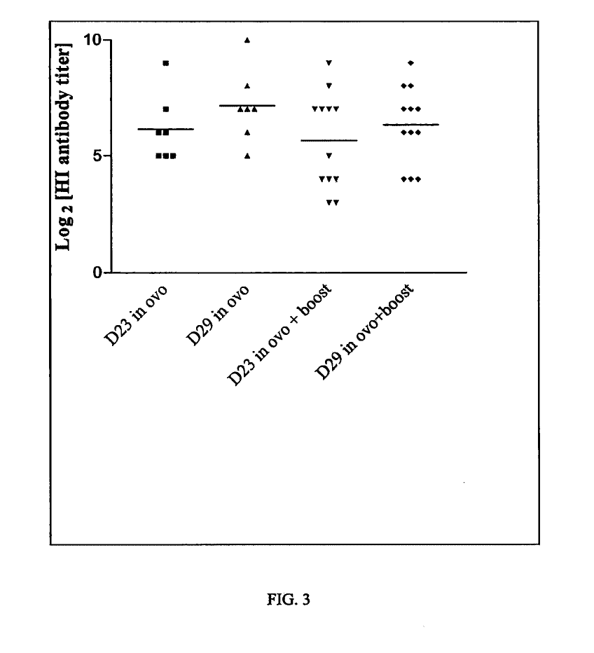

19 SPF chicken embryos were immunized by the in ovo route at day 18 of incubation with the same dose of AdTW68.H5 as described in Example 7. Hatched chickens were individually identified by wing band. A group of 12 chickens was boosted via the nasal route at day 15 after hatch and the remaining 7 chickens were not boosted. Blood samples were obtained from each wing-banded bird at days 23 and 29 of age and tested by HI for antibodies against avian influenza strain A/turkey/Wisconsin/68. Overall, the HI antibody titers detected in these birds (FIG. 3) were similar to the values obtained in the previous trial (FIG. 2). Most birds achieved titers ≧5 log₂. Chicks inoculated only in ovo achieved titers between 5 and 9 log₂ at day 23 post-hatch (FIG. 3). Those chickens either maintained or increased their antibody titers by day 29 post-hatch. In ovo vaccination in conjunction with intranasal booster showed antibody titers varying between 3 and 9 log₂ by day 23 post-hatch (FIG. 3). Similarly as in the previous group, most chicks had increased their titers by 1 or 2 log₂ steps by day 29 post-hatch.

Challenge was performed in biosafety level 3+ facilities by oro-pharyngeal inoculation with 10⁵ embryo infective doses (EID₅₀) of the HPAI A/chicken/Queretaro/19/95 (H5N2) (Horimoto, 1995, Garcia, 1998). The H gene of this challenge strain has 90.1% nucleotide identity and 94.4% deduced amino acid similarity with the H of AI strain A/turkey/Wisconsin/68 used in the Ad-vectored vaccine (GenBank accessions U79448 & U79456) (SEQ ID NOs: 5, 6, 7, 8).

A total of 30 chickens, including 7 chicks vaccinated in ovo and 12 chicks vaccinated in ovo and subsequently boosted intranasally at day 15 post-hatch, as well as 11 unvaccinated controls, were challenged at day 34 post-hatch.

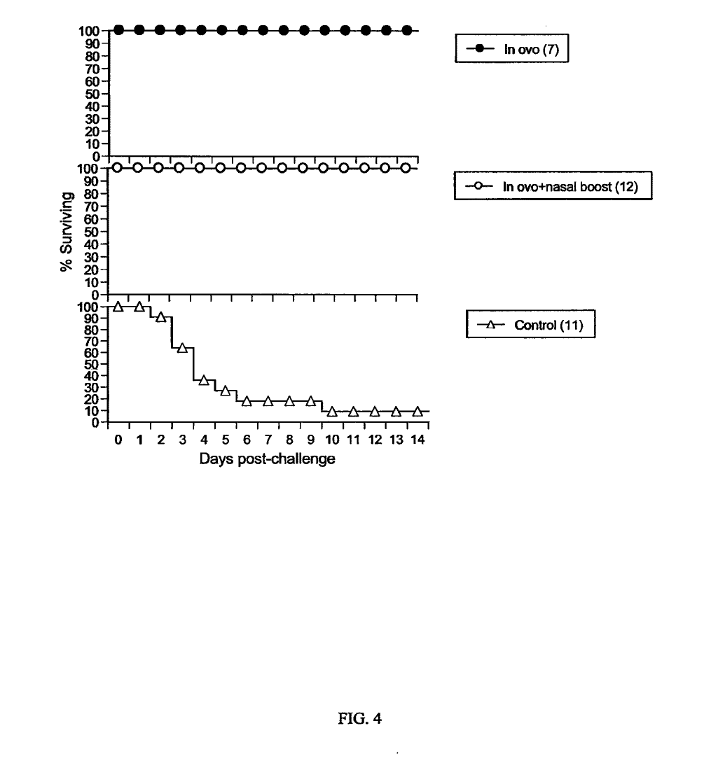

Challenged birds were observed daily for morbidity and mortality throughout an experimental period of 14 days. Clinical signs of AI, including swelling of comb and wattles, conjunctivitis, anorexia and hypothermia, were observed two days post-challenge in 10 of 11 control birds. Two days later, most survivors in the control group exhibited comb necrosis, swelling of wattles, diarrhea, dehydration, lethargy, and subcutaneous hemorrhages of the leg shank. No signs of disease were developed in any of the vaccinated birds. All birds vaccinated with the AdTW68.H5 (19/19) (in ovo only and in ovo+nasal boost) survived the challenge (FIG. 4).

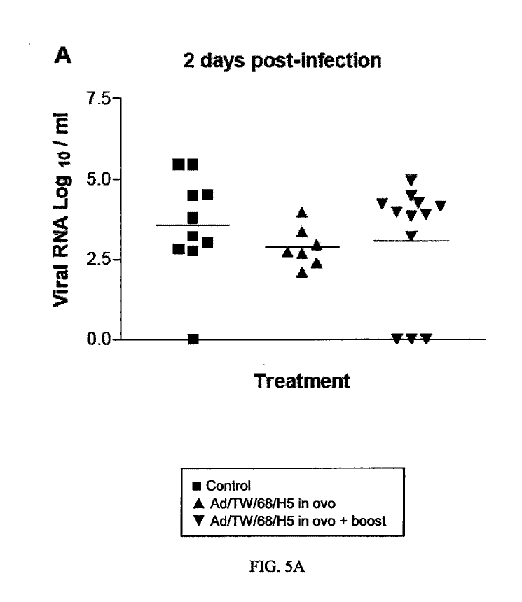

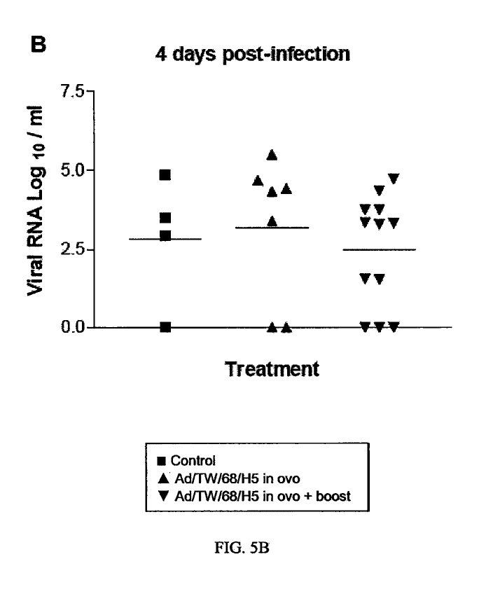

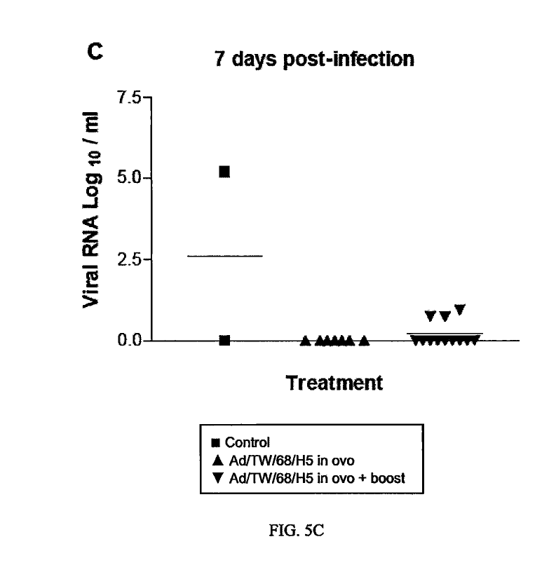

Viral genomes of the A/chicken/Queretaro/19/95 in challenged birds were quantitatively determined by real-time reverse transcriptase-polymerase chain reaction (RT-PCR) in oropharyngeal swabs collected 2, 4, and 7 days post-challenge. There was a significant difference (P<0.05) in the concentration of AI viral genomes between vaccinated and unvaccinated chickens 7 days after challenge (FIG. 5). Absence of detectable viral RNA in immunized birds provides evidence that in ovo vaccination elicited an immune response capable of controlling AI virus shedding within a week.

These results collectively show that chickens immunized in ovo with RCA-free human Ad vectors encoding H genes from different influenza viruses (human and avian origin) developed HI antibody titers against the homologous AI virus, and were protected against lethal challenge with a highly pathogenic AI virus strain of the same H type.

### Example 9

### In Ovo Inoculation of AdTW68.H5 Protects Against Lethal Challenge with the Highly Pathogenic Avian Influenza Strain A/swan/Mongolia/244L/2005 (H5N1)

To determine whether the AdTW68.H5-vectored AI vaccine can confer protection against a recent H5N1 HPAI virus strain, 31 chickens were vaccinated in ovo with the AdTW68.H5 vector at a dose of 2×10⁸ ifu. Control groups included 10 chickens vaccinated with an Ad5 vector (AdCMV-tetC) encoding an irrelevant antigen (tetanus toxin C-fragment) (Shi et al., 2001) and 10 chickens which were not exposed to Ad5 vectors.

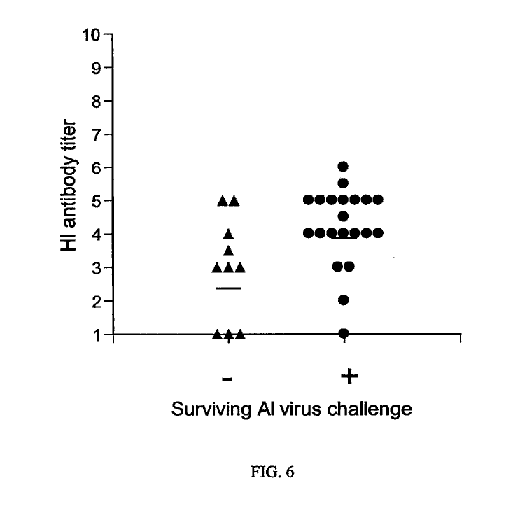

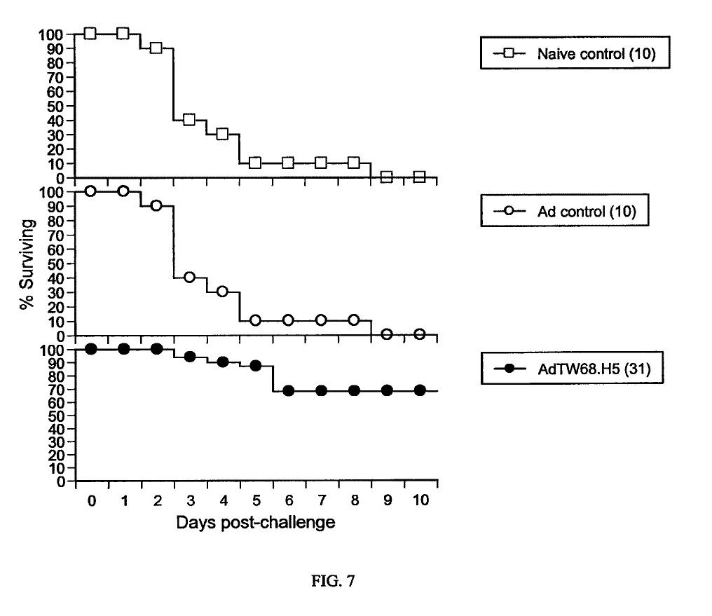

On D31, control and immunized chickens were challenged with the H5N1 AI virus A/swan/Mongolia/244L/2005 (the HA of this challenge strain has 89% deduced HA amino acid sequence similarity with the HA of the A/turkey/Wisconsin/68 strain). As shown in FIG. 6, in ovo immunization induced antibodies within a range of 1 and 6 log₂ on D25. None of the unvaccinated (10/10) and AdCMV-tetC-immunized (10/10) birds produced measurable HI antibodies and all died from AI within 9 days post-challenge, whereas 68% (21/31) of the AdTW68.H5-vaccinated birds survived without clinical signs 10 days after the challenge (FIG. 7). Notably, 7 birds in the immunized group with HI titers of ≧3 log₂ (FIG. 6) were still killed by this highly lethal H5N1 AI virus. It is likely that the survival rate against this H5N1 AI virus may be improved by in ovo vaccination with an Ad5 vector encoding an HA with closer antigenicity.

These results demonstrate that chickens immunized in ovo with an RCA-free human Ad5 vector encoding avian H5 HA could elicit protective immunity against HPAI viruses.

### Example 10

### Construction of an Adenovirus Vector Encoding the A/chicken/New York/13142-5/94 Hemagglutinin

An E1/E3-defective human adenovirus serotype 5 (Ad5)-derived vector encoding the avian influenza (AI) virus strain A/chicken/New York/13142-5/94 H7 hemagglutinin (HA) was generated as described previously (Toro et al., 2008). The full-length H7 HA gene of the AI virus was inserted into the shuttle plasmid pAdApt to generate the plasmid pAdApt-NY94.H7. A replication-competent adenovirus (RCA)-free Ad5 vector (AdChNY94.H7) encoding this H7 HA was generated by co-transfection of PER.C6 cells with pAdApt-NY94.H7 and the Ad5 backbone plasmid pJ M17, followed by multiple cycles of plaque purification after the cytopathic effect (CPE) appeared.

### Example 11

### Immunization of Chickens by Aerosol Spray of the AdChNY94.H7 Vector

One-day-old chickens were separated into three groups, each of which was administered a composition via aerosol spray. Group A (n=15) was immunized by aerosol spray of the AdChNY94.H7-vectored AI vaccine (as prepared in Example 10) in a volume of 8 ml containing 1.1×10¹⁰ infectious units (ifu) per ml in a single-dose regimen. Group B (n=15) was immunized by aerosol spray of the same vaccine in a volume of 24 ml (also 1.1×10¹⁰ ifu of Ad5 per ml) and additionally received a booster application on day 16 of age. Group C was the unvaccinated control.

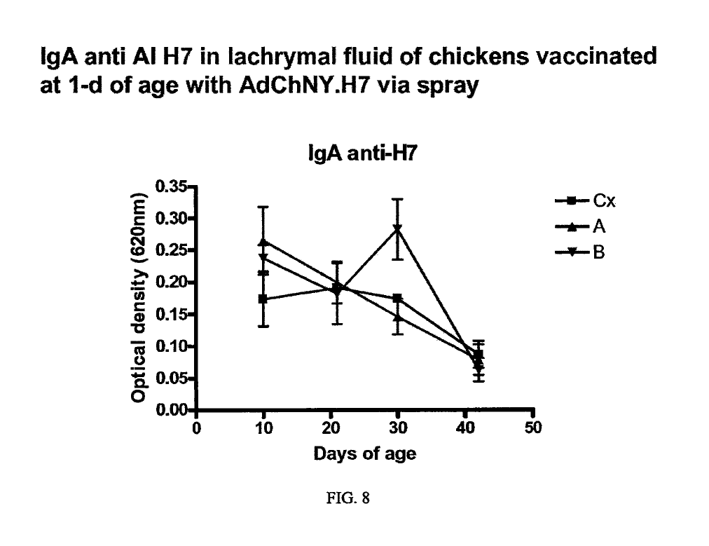

FIG. 8 shows specific IgA levels in lachrymal fluid as measured by ELISA.

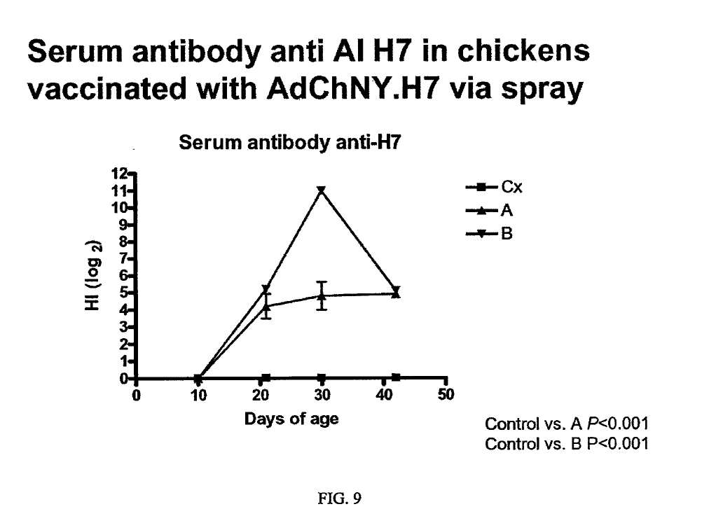

FIG. 9 shows hemagglutination-inhibition (HI) antibody titers in chicken sera.

As can be seen from the figures, both immune responses were elicited in both Groups

A and B, with the members of Group B demonstrating an increase in immune response following the booster dose on day 16. This demonstration that aerosol spray of human Ad5-vectored vaccines was effective in vaccinating chickens may be attributed to intraocular administration, a combination of multiple routes (intranasal, intraocular, transdermal, and oral administration), and/or the fine mist generated during aerosol spray.

### Example 12

### Induction of Mucosal Immunity in the Avian Harderian Gland with a Replication-Deficient Human Adenovirus Vector Encoding an Avian Influenza H5 Hemagglutinin

Chickens

Specific pathogen free (SPF) white leghorn chicken fertilized eggs (Charles River Laboratories, North Franklin, Conn.) were incubated and hatched. Chickens were maintained under BSL2 conditions in Horsfall-type isolation units throughout the experimental period. Experimental procedures and animal care were performed in compliance with federal and institutional animal care and use guidelines.

RCA-free Ad-vectored AI vaccine.

The RCA-free E1/E3-defective AdTW68.H5_{ck} vector encoding a codon-optimized H5 HA of the A/turkey/Wisconsin/68 AI virus under transcriptional control of the cytomegalovirus (CMV) early promoter was generated in PER.C6 cells (provided by Crucell Holland BV).

Ocular Vaccination

In order to induce H5-specific immunity 9 or 10-day-old SPF White leghorn chickens (Charles River Labs) were vaccinated via the ocular route with 2.5×10⁸ ifu of AdTW68.H5_{ck} per bird in a volume of 80-100 μl. In specific experiments the chickens were boosted twice at intervals of 14 days with the same vaccine dose via the ocular route. The control group was either naïve birds or birds vaccinated with Ad5 expressing an irrelevant protein (tetanus toxin C fragment).

Sample Collection

Blood samples were collected after puncturing the brachial vein, allowed to clot and serum was obtained by centrifugation for 1 min. at 4,500×g. The serum was collected and A 10× protease inhibitor cocktail containing [4-(2-aminoethyl)benzenesulfonyl fluoride hydrochloride, aprotinin, bestatin hydrochloride, [N-(trans-epoxysuccinyl)-L-leucine 4-guanidinobutylamide], EDTA, leupeptin (Sigma, Saint Louis, Mo.) was added to the sera prior to storage at 4° C. or for long-term storage at −80° C. Lachrymal fluid was obtained as previously described (Toro, 1996), centrifuged for 5 min. at 3,300×g and mixed with a 10× protease cocktail, stored at 4° C. or long-term at −80° C. Serum and lachrymal fluid were collected at days 17, 40, 50, 63 and 68 of age for antibody determinations.

Antibody Measurements

Serum AI H5 antibody levels were measured by hemagglutination inhibition (HI) assay against 4 hemagglutinating units of the low pathogenic A/turkey/Wisconsin/68 (H5N9) strain. Titers of <1.0 Log₂ were arbitrarily assigned a titer of 1.0. No HI antibodies were detected in control chickens.

Serum and lachrymal fluids were analyzed by ELISA for Ad5-specifc IgA and IgG levels. The ELISA for Ad5 was performed as previously reported (van Ginkel) except that HRP-conjugated goat-anti chicken IgA, IgG and IgM antibodies (Gallus Immunotech Inc., Fergus, Canada) were used as detection antibodies. ELISA plates were coated with 10⁸ particles/well of wild-type Ad5. The wells were blocked and serial two-fold dilutions of the samples were added and incubated overnight at 4° C. Horseradish peroxidase (HRP) conjugated goat anti-chicken IgA or IgG antibodies (Gallus Immunotech Inc., Fergus, Canada) were used to detect Ad5-specific antibodies. The wells were washed and substrate was added. After 30 minutes at room temperature the reaction was stopped and the absorption at 405 nm was measured. The highest dilution with an OD₄₀₅ of 0.100 or more above background was defined as the endpoint-titer.

Chicken B Cell Enzyme-Linked Immunospot (ELISPOT) Assay

Harderian glands and spleens were collected from these chicks on days 20, 30, 40, 50, and 60 of age in order to measure the number of IgA and IgG antibody secreting cells specific for Ad5 or H5 (coated with A/tk/WI/68 AI strain) using an ELISPOT assays as described (Czerkinsky). In brief, the HGs were mechanically disrupted and lymphocytes were isolated by centrifugation over a 1.077 g/ml histopaque-ficoll density gradient. The isolated lymphocytes were counted on a hemocytometer using trypan blue exclusion. The lymphocytes were loaded at various concentrations onto nitrocellulose backed, 96-well microplates coated with UV killed avian influenza virus (A/tk/WI/68) at 2× the hemagglutination titer or heat-killed Ad5 virus (10⁸ particles/well) and blocked with complete RPMI-1640 medium containing 10% fetal calf serum (FCS). The cells were incubated for approximately 18 hrs at 37° C. in a humidified incubator with 5% CO2. The plates were washed 5× with PBS-Tween 20 (0.05%) and incubated overnight at 4° C. with goat-anti-IgG or goat-anti-IgA conjugated to HRP (Gallus Immunotechnology, Inc.). The plates were washed and incubated at room temperature for 15-30 minutes with peroxidase substrate (Moss Inc.) prior to stopping the reaction by washing the plates with water.

Immunoprecipitation

Tears or serum was incubated overnight at 4° C. with 16.5 μl biotinylated mouse-anti-chicken IgA monoclonal antibody (Southern Biotechnology Associates, Inc., Birmingham, Ala.) or 16 μl biotinylated goat-anti-chicken IgA (Southern Biotechnology Associates, Inc.). The next day 50 μl washed Streptavidin-conjugated Sepharose beads (GE Healthcare Bio-Sciences AB, Uppsala, Sweden)) were added overnight at 4° C. while continuously agitated on a shaker. The next day the beads were precipitated by centrifugation and washed 3× with 1 ml of PBS with 0.05% Tween20 and one final wash with PBS. A 2× Tris-Glycine SDS sample buffer (Invitrogen, Carlsbad, Calif.) was added and the samples were boiled for 10 min. at 100° C. The samples were subsequently centrifuged to remove the beads and the supernatants were analyzed by SDS-polyacrylamide gel electrophoresis (SDS-PAGE) on a 4-12% pre-cast gradient gel (Invitrogen). The proteins were visualized using the Silver SNAP® silverstain kit according to manufacturer's recommendations (Pierce, Rockford, Ill.).

Immunohistochemistry

White leghorns were exposed to 2.5×10⁸ ifu of AdTW68.H5_{ck}. The tissues were fixed in acidic acetate-alcohol for two hours at 4° C. followed by incubation in sucrose (30%) in PBS to cryoprotect the tissues, after which the tissues were embedded in Neg 50 medium (Richard-Allan Scientific, Kalamazoo, Mich.) and frozen into an embedding mold (Fisher Scientific) in isopentane (Fisher Scientific) cooled with liquid nitrogen. Five μm sections were cut on a cryostat (Microm HM550, Waldorf, Germany) and were placed on precleaned, Superfrost® Plus microscope slides (Labsco, Inc., Louisville, Ky.) and allowed to dry. The slides were treated to remove lipids with ice cold acetone for 4 minutes, dried, blocked with 10% FCS for 1 hr at room temperature, followed by incubation overnight at 4° C. with an anti-H5 affinity-purified rabbit-anti H5 antibody (eEnzyme LLC, Gaithersburg, Md.) diluted in 10% FCS. This step was followed by incubation with biotinylated Donkey-anti-rabbit IgG (R&D Systems) at 4° C. overnight and a final staining step with Neutralite Avidin-FITC (Southern Biotechnology Associates, Inc., Birmingham, Ala.) in 1% FCS for 4 hours at room temperature. Between steps the slides were extensively washed in PBS and cover glasses were mounted with Vectashield Hard Set mounting medium (Vector Laboratories, Inc., Burlingame, Calif.). All images were captured in gray scale using SPOT software (Diagnostic Instruments) and edited in Microsoft Office Picture Viewer. The pictures were colored green by using the following settings; “amount” adjusted to 70, “hue” adjusted to 100, “saturation” adjusted to 100.

RT-PCR Chicken Polymeric Immunoglobulin Receptor

Total RNA was isolated from the Harderian glands of three chickens using Tri-reagent (Molecular Research, Inc.) according to the manufacturer's protocols. One microgram of total RNA was reverse transcribed and amplified by 35 PCR cycles of 94° C., 1 min., 58° C., 1 min to detect expression of the chicken polymeric immunoglobulin receptor (pIgR). The forward primer starts at nucleotide 206 3′-CCAGGAGTTGCTTGACTGT-5′ and the reverse primer starts at nt 605 3′-CTCAGCAGGATTCTCCCTTG-5′, thus, when separated on a 1.5% agarose gel and stained with ethidium bromide a 400 bp diagnostic PCR product will be observed if pIgR mRNA is amplified. Since the primers cover an intron an approximately ˜750 bp amplicon was seen after genomic DNA amplification. To eliminate potential DNA contamination the RNA was treated with RNase-free DNase (Sigma).

Statistical Analysis

All statistical analyses were performed using the Student's unpaired, two-tailed t test.

Results

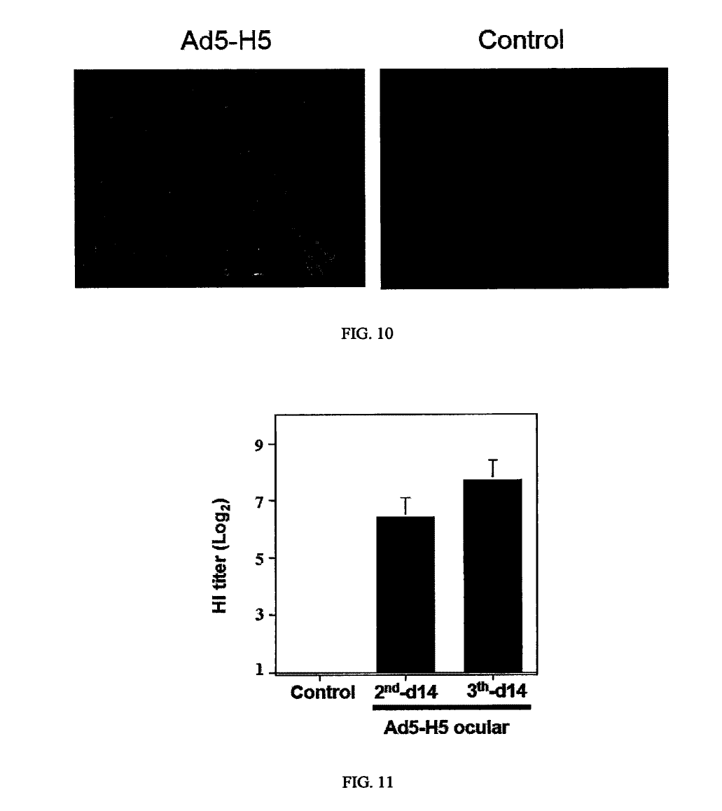

It was demonstrated that expression of the H5 HA from AdTW68.H5_{ck} could be detected in HG 9 days post-ocular immunization (FIG. 10) using immunofluorescent staining. The H5 expressing cells aligned to a large extent to the canals present in HGs. No expression of H5 was noted in control tissues. Compelling evidence shows that the human Ad5 is able to transduce specific chicken cells in vivo.

To measure the systemic antibody response to AI virus the HI assay was performed on serum samples. Serial dilutions were made from the sera collected 2 weeks after the second and third ocular administration of AdTW68.H5_{ck}. As shown in FIG. 11, a strong HI antibody response was observed both after the second AdTW68.H5_{ck} application (HI titer 6.4, n=15) and the third application (HI titer 7.7, n=9), while no HI titer was detected in any of the uninoculated controls (HI=0, n=11).

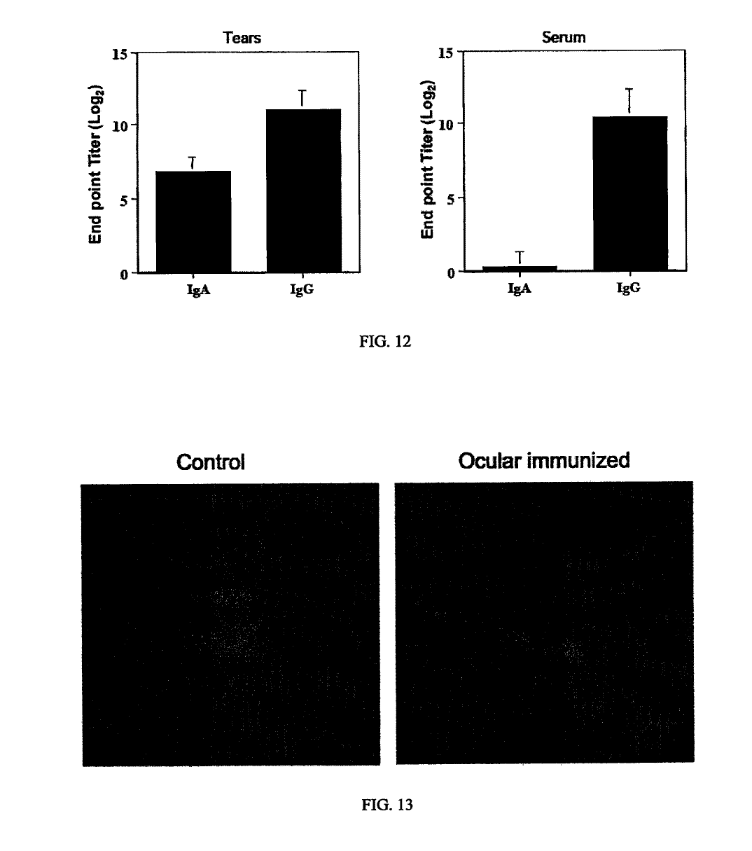

ELISA revealed high IgA and IgG Ad5-specific antibodies in tears, while only high IgG titers were detected in serum (FIG. 12). No IgA or IgG antibodies were detected in control chickens when starting at 2× diluted samples for IgA detection or 32× diluted samples for IgG detection (data not shown). The IgG levels in tears and sera were not significantly different (P=0.4072). This could indicate that IgG antibodies in tears were produced in the HGs or were derived from serum exudates or more likely were derived from a combination of these two. Elevated IgA levels were observed in tears (mean Log₂ endpoint titer of 6.8) while IgA serum levels were undetectable in most chickens (mean Log₂ endpoint titer of 0.3). The IgA levels in tears were significantly higher than those observed in serum (P<0.0001). This indicated that most IgA antibodies are locally produced in the HGs after ocular immunization. In order to confirm this and also determine IgG production in the HGs, we developed a chicken ELISPOT assay.

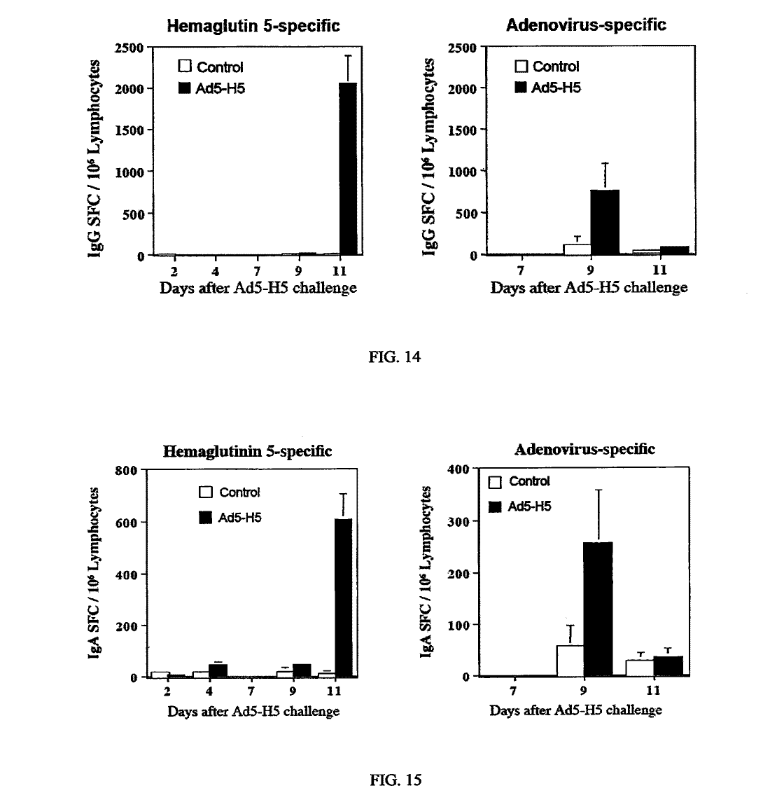

IgA and IgG ELISPOT assays were developed for the Ad5 vector and the expressed H5 HA as described in detail in the Material and Methods. Illustrated in FIG. 13 are the IgA spot forming cells (SFC) observed after ocular challenge in the HG. The IgG SFC response specific for Ad5 and H5 induced in the HG after ocular challenge are shown in FIG. 14. The Ad5-specific immune response peaks at 9 days following ocular administration with 752 SFC/10⁶ lymphocytes. The peak IgG response to the H5 HA was 2 days later, 11 days after AdTW68.H5_{ck} administration and is 2,047 SFC/10⁶ lymphocytes. This is 2.7× higher in magnitude than the Ad5 IgG SFC response (FIG. 14). The same delay of 2 days was observed for the H5 IgA SFC response (FIG. 15) when compared with the Ad5 response and is 2.4× higher than the Ad5 IgA SFC response. H5 and Ad5 peak IgA responses were 605 SFC/10⁶ lymphocytes and 257 SFC/10⁶ lymphocytes, respectively.

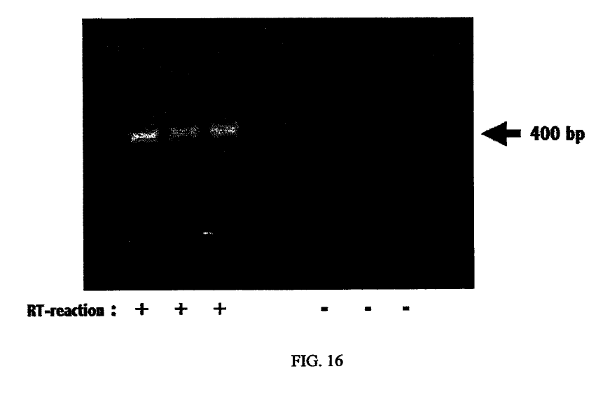

Since the HGs are located adjacent to the eye they are perfectly located to form the first line of defense against invading pathogens by secreting polymeric IgA. Although the chicken polymeric immunoglobulin receptor (pIgR) has been cloned and its expression has been analyzed in various tissues (Wieland, 2004) as well as its association with polymeric IgM and IgA has been demonstrated (Kobayahi, 1980), no data is available on the expression of this essential receptor to generate mucosal antibody responses in the chicken HGs. Total RNA isolated from the HGs was analyzed by RT-PCR after treatment with RNase-free DNase to eliminate potential DNA contamination. As shown in FIG. 16, the pIgR was expressed in the HGs. No PCR product was observed when reverse transcription of the RNA was left out. Furthermore, amplification of pIgR DNA would result into a ˜750 bp product, which was not observed. This observation confirms that pIgR mRNA was expressed in the HGs. In addition, the RT-PCR product was sequenced at the Auburn University sequencing facility and was found to be identical to the published chicken pIgR mRNA sequence (Wieland, 2004).

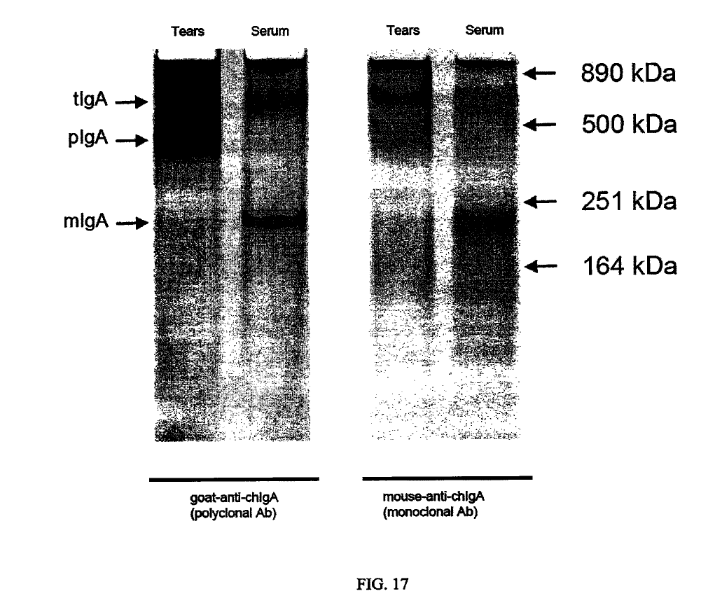

To confirm that expression of pIgR in the HGs resulted in secretion of polymeric IgA in tears, immunoprecipitations were performed of both tears and serum IgA. The proteins isolated by this procedure were analyzed on SDS-PAGE and visualized using the Silver SNAP® stain kit (FIG. 17) according to manufacturer's recommendations (Pierce, Rockford, Ill.). Three different size proteins were precipitated with mouse monoclonal antibodies to chicken IgA. The smallest protein had an estimated molecular weight between ˜200-230 kDa, which was consistent with a monomeric IgA (mIgA) molecule, composed of two light chains and two heavy chains. A clear difference in abundance of mIgA in tears and serum proteins was observed. The mIgA was the most prevalent form of IgA in serum and the least prevalent in tears. The next highest molecular weight (MW) protein with an estimated MW of ˜470 kDa is the most abundant protein in tears and the least abundant protein in serum. This protein was based on size (Wieland, 2004; Watanabe, 1975; Watanabe, 1974), dimeric IgA associated with the secretory component (SC) of the pIgR. The largest protein precipitated was based on size (Watanabe, 1975; Watanabe, 1974), presumably tetrameric IgA (tIgA), with an estimated MW of ˜710 kDa. The tIgA was abundant in both tears and serum. This data demonstrated that monomeric IgA was most prevalent in serum while dimeric IgA was most prevalent in mucosal secretions such as tears. This scenario was identical to that observed in mammals.

Thus, these results demonstrate that the human replication-deficient AdTW68.H5_{ck} vaccine vector; 1) effectively transduced HG's cells upon ocular exposure, which 2) resulted in both systemic and mucosal immune responses. In addition, this is the first quantitative analysis of antibody secreting cells in the chicken HGs using the ELISPOT assay and the first report on the use of the IgA ELISPOT in chicken. Induction of IgG and IgA responses to both the H5 transgene and the Ad5 vector were observed. This combined with the finding that HGs expressed the pIgR and secreted predominantly pIgA in tears, confirmed the importance of the HGs for generating protective immune responses to pathogens in both the systemic and mucosal compartments.

The question of how a human Ad5 vector transduced chicken cells, still remains to be solved. It is likely that chickens do not express the traditional Coxackie-Adenovirus-Receptor (CAR), which is known to interact with the Ad5 fiber knob (Bergelson, 1997), based on the fact that wild-type human Ad5 is not an avian pathogen. Other mechanisms of Ad5 to transduce cells other than through the CAR receptor have been proposed. For example, MHC class I may contain a CAR mimotope that may be involved in Ad5 uptake (Hong, 1997). In addition, the penton base proteins have a RGD motif, which promotes internalization of adenovirus by αv-integrins (Wickham, 1993; Davison, 2001). Whether or not integrins could play a role in transduction of chicken cells with Ad5 in the absence of the CAR receptor, as has been reported for shorter shafted adenoviruses (Roelvink, 1998), is not known. Ad5 transduced dendritic cells (DCs) in vitro at a relative low efficiency due to a lack of CAR receptor expression on these cells (Okada, 2001; Rea, 1999). A recent study, comparing Ad5 and Ad35 (known to target DCs through CD46) for their ability to transduce DCs after intradermal delivery, demonstrated that Ad35 was approximately 2× more efficient in transducing DCs than Ad5, however; a considerable percentage of DCs, including CD83⁺ mature DCs, were transduced by Ad5 (de Gruijl, 2006). Considering the ability of DCs to function as potent inducers of adaptive immune responses, we speculate that in the absence of CAR receptor, the presumed scenario in chickens, the Ad5 could be more prone to target DCs. Targeting DCs may further increase upon boosting the Ad5-specific immune response, since IgG antibodies to Ad5, induced during priming, may opsonize the Ad5 and target them to CD64⁺ (high affinity Fcγ-R1 receptor) on DCs. A study, targeting the Ad5 vector to CD64 demonstrated a 10-15 fold increase of human DCs transduction compared to unmodified Ad5 (Sapinoro, 2007). It is interesting in this context, that H5-expressing cells in the HGs after ocular Ad5-H5 administration, seemed to line to a large extent the draining canals of the HGs, a similar distribution recently reported for CD83⁺DCs in chicken HGs (Hansell, 2007).

Only two publications pertaining to the B cell ELISPOT assay in chickens have been published. The first was published in 1998 as a research note in Poultry Science and demonstrated IgG and IgM spot-forming cells (SFC) specific for infectious bursal disease virus (IBDV) in the spleen (Wu, 1998) and formed the proof of concept that the ELISPOT was a valuable and sensitive tool to dissect the immune responses in chickens. The second publication was a more elaborate study, in which a more detailed analysis of the dynamics of infectious bronchitis virus-specific IgG SFC in peripheral blood mononuclear cells and spleen was performed (Pei, 2005). Our analyses involved both IgG and the IgA SFC specific for Ad5 and AI H5 in the HGs. No previous analyses of IgA SFC in chicken have been reported nor has there been any analysis of SFC induced in the HGs. The Ad5-H5 induced H5-specific IgA and IgG responses in the HGs were approximately 2.5× higher than the Ad5-specific responses. The IgG SFC responses in the HGs were approximately 3× higher than the IgA SFC responses to H5 and Ad5. It is possible that some of the IgG SFC responses originated from blood-derived lymphocytes, since we did not perfuse the HGs with PBS prior to isolation of lymphocytes. Nevertheless, a strong IgG response was observed in the HGs, which was also reflected by Ad5-specific IgG antibody levels in tears. The 2 days delay in the H5-specific antibody SFC response compared to the Ad5-specific response would argue that there is a delay between exposure to the Ad5 vector and expression of the H5 transgene. It is not clear why this delay would occur and this observation was different from what has been shown in mice using Ad5 (van Ginkel, 1995). Whether or not this is related to the route of infection by the Ad5 vector of chicken cells, remained to be determined.

IBV-specific lachrymal IgA levels are related to the level of resistance to ocular IBV challenge (Toro, 1994). This illustrates the importance of IgA antibodies in tears for protection of mucosal surfaces to exposure to pathogens. From the applied point of view, these findings are relevant as they prove the feasibility of vaccinating existing (adult) chicken populations against AI with the same adenovirus recombinant technology previously reported to effectively protect chickens after in ovo vaccination.

Although the avian J chain has been cloned and was demonstrated to be expressed in HG B cells (Takahashi, 2000) limited analyses of the molecular composition of tears-derived IgA exist. Watanabe et al. reported secretion of a tetrameric-IgA (tIgA) in tears with an approximately molecular weight of 650 kDa and this tIgA was not associated with a secretory component (SC) of the polymeric immunoglobulin receptor (pIgR) (Watanabe, 1975). The intestines were the only organ in which IgA secretion was associated with SC (Wieland, 2004; Watanabe, 1975; Watanabe, 1974). The molecular weight of this polymeric IgA (pIgA) was estimated between 350-500 kDa (van Ginkel, 1995; Watanabe, 1975; Watanabe, 1974). The cloning of chicken pIgR enabled the demonstration of pIgR mRNA by Northern blot analyses. The mRNA was not only expressed in the intestines but also in liver, thymus and bursa of Fabricius. In contradiction with previous reports (Watanabe, 1975), SC was also demonstrated to associate with bile-derived IgA (Wieland, 2004). Based on the analysis of immunoprecipitated IgA from tears and serum of chickens, at least three different forms of IgA are present in chickens. Based on molecular weights a monomeric form of IgA is most prevalent in serum and almost absent in tears. A dimeric IgA, presumably associated with the SC based on molecular weight, was the most prevalent form in tears when compared to serum. Finally, a multimeric, presumably tIgA was present in both tears and serum at approximately equivalent levels.

In addition, it was demonstrated by RT-PCR that expression of the pIgR in chicken HGs occurred. A lack of pIgR expression in the HGs would have questioned the function of pIgR in chicken. This finding is consistent with a role of the pIgR for transporting pIgA across a mucosal epithelium into tears and high prevalence of pIgA in mucosal secretions of 25 chickens but not in serum. In serum monomeric IgA (mIgA) prevails. This same scenario was found in mammals. Thus, the lack of a hinge region in chicken IgA (Mansikka, 1992), the region most susceptible to proteolysis in mammals, did not alter the association of pIgA with the SC in mucosal secretions. If association with SC did not provide the avian pIgA more protection against proteases, it raised the question whether or not its sole function was 30 transport of pIgA across the epithelium. It was interesting in this context that both dimeric IgA and pentameric mammalian IgM were transported across the mucosal epithelium by the pIgR. Pentameric IgM required a J-chain for proper assembly, while hexameric IgM did not contain a J-chain (Randall, 1990; Wiersma, 1998). The J-chain is highly conserved in chicken when compared to mammals (Takahashi, 2000) and contained two conserved regions, which were important for interaction with the pIgR (Braathen, 2007). The lack of the J-chain in hexameric IgM makes it ˜20-fold more efficient than J-chain containing pentameric IgM in complement fixation (Randall, 1990; Wiersma, 1998). Thus, it could be hypothesized that the J-chain in combination with the SC were bound to polymeric antibodies in order to prevent activation of complement, inflammation and associated tissue damage at mucosal surfaces. These functions may be more important for IgM than IgA, since mammalian IgA was inherently poor in activating complement. No data was available whether these antibodies would play a similar role at avian mucosal surfaces.

The tIgA found in both serum and tears of chicken may account for differences in size reported by different investigators for bile- and tears-derived IgA in chicken (Kobayashi, 1980, Watanabe, 1975; Watanabe, 1974), which were reported not to contain SC (Kobayashi, 1980, Watanabe, 1975). IgA in mucosal secretions of humans also contained tetrameric and trimeric forms, but these forms bound to the pIgR and contained the SC of this receptor (Song, 1995). It is possible that chicken IgA is different from mammalian IgA in this respect, and the mechanism as to how tIgA is transported into mucosal secretion such as tears, bile and saliva of chicken requires further analysis. In addition, these data demonstrated the importance of the HGs to generate both mucosal and systemic immunity following ocular exposure and prove the feasibility of eyedrop vaccination of adult chickens against avian pathogens with human Ad5-vectored vaccines.

Having thus described in detail embodiments of the present invention, it is to be understood that the invention defined by the appended claims is not to be limited by particular details set forth in the above description as many apparent variations thereof are possible without departing from the spirit or scope thereof.

The invention will now be further described by the following numbered paragraphs:
1. A method of inducing an immune response in an animal, comprising administering an effective amount of an immunogenic or vaccine composition such that a protective immune response is induced in the animal, wherein the administration is via aerosol spray.
2. The method of paragraph 1, wherein immunogenic or vaccine composition comprises a recombinant human adenovirus expression vector that comprises and expresses an adenoviral DNA sequence, and a promoter sequence operably linked to a foreign sequence encoding one or more avian antigens or immunogens of interest.
3. The method according to paragraph 2, wherein the adenoviral DNA sequence is derived from adenovirus serotype 5 (Ad5).
4. The method according to paragraph 2, wherein the adenoviral DNA sequence is selected from the group consisting of replication-defective adenovirus, non-replicating adenovirus, replication-competent adenovirus, and wild-type adenovirus.
5. The method according to paragraph 4, wherein the adenoviral DNA sequence is E1/E3 deleted.
6. The method according to paragraph 2, wherein the promoter sequence is selected from the group consisting of viral promoters, avian promoters, CMV promoter, SV40 promoter, β-actin promoter, albumin promoter, Elongation Factor 1-α (EF1-α) promoter, PγK promoter, MFG promoter, and Rous sarcoma virus promoter.
7. The method according to paragraph 2, wherein the foreign sequence encoding the one or more avian antigens or immunogens of interest is derived from avian influenza virus, infectious bursal disease virus, Marek's disease virus, avian herpesvirus, infectious laryngotracheitis virus, avian infectious bronchitis virus, avian reovirus, avipox, fowlpox, canarypox, pigeonpox, quailpox, and dovepox, avian polyomavirus, Newcastle Disease virus, avian pneumovirus, avian rhinotracheitis virus, avian reticuloendotheliosis virus, avian retroviruses, avian endogenous virus, avian erythroblastosis virus, avian hepatitis virus, avian anemia virus, avian enteritis virus, Pacheco's disease virus, avian leukemia virus, avian parvovirus, avian rotavirus, avian leukosis virus, avian musculoaponeurotic fibrosarcoma virus, avian myeloblastosis virus, avian myeloblastosis-associated virus, avian myelocytomatosis virus, avian sarcoma virus, or avian spleen necrosis virus.
8. The method according to paragraph 7, wherein the foreign sequence encoding the one or more avian antigens or immunogens of interest is derived from one or more avian viruses.
9. The method according to paragraph 7, wherein the foreign sequence encoding the one or more avian antigens or immunogens of interest is derived from avian influenza virus.
10. The method of paragraph 7, wherein the foreign sequence encoding the one or more avian antigens or immunogens of interest is selected from the group consisting of genes encoding hemagglutinin, spike protein, other external proteins, nucleoprotein, matrix, neuraminidase and genes coding non-structural proteins such as enzymes or other regulatory proteins.
11. The method of paragraph 7, wherein the foreign sequence encoding the one or more avian antigens or immunogens of interest is selected from the group consisting of hemagglutinin subtype 3, 5, 7, and 9.
12. An immunogenic composition or vaccine for aerosol spray delivery to an avian subject comprising a veterinarily acceptable vehicle or excipient and a recombinant human adenovirus expression vector that comprises and expresses an adenoviral DNA sequence, and a promoter sequence operably linked to a foreign sequence encoding one or more avian antigens or immunogens of interest.
13. The immunogenic composition or vaccine of paragraph 12, wherein the adenoviral DNA sequence is derived from adenovirus serotype 5 (Ad5).
14. The immunogenic composition or vaccine of paragraph 12, wherein the adenoviral DNA sequence is selected from the group consisting of replication-defective adenovirus, non-replicating adenovirus, replication-competent adenovirus, and wild-type adenovirus.
15. The immunogenic composition or vaccine of paragraph 14, wherein the adenoviral DNA sequence is E1/E3 deleted.
16. The immunogenic composition or vaccine of paragraph 12, wherein the promoter sequence is selected from the group consisting of viral promoters, avian promoters, CMV promoter, SV40 promoter, β-actin promoter, albumin promoter, Elongation Factor 1-α (EF1-α) promoter, PγK promoter, MFG promoter, and Rous sarcoma virus promoter.
17. The immunogenic composition or vaccine of paragraph 12, wherein the foreign sequence encoding the one or more avian antigens or immunogens of interest is derived from avian influenza virus, infectious bursal disease virus, Marek's disease virus, avian herpesvirus, infectious laryngotracheitis virus, avian infectious bronchitis virus, avian reovirus, avipox, fowlpox, canarypox, pigeonpox, quailpox, and dovepox, avian polyomavirus, Newcastle Disease virus, avian pneumovirus, avian rhinotracheitis virus, avian reticuloendotheliosis virus, avian retroviruses, avian endogenous virus, avian erythroblastosis virus, avian hepatitis virus, avian anemia virus, avian enteritis virus, Pacheco's disease virus, avian leukemia virus, avian parvovirus, avian rotavirus, avian leukosis virus, avian musculoaponeurotic fibrosarcoma virus, avian myeloblastosis virus, avian myeloblastosis-associated virus, avian myelocytomatosis virus, avian sarcoma virus, or avian spleen necrosis virus.
18. The immunogenic composition or vaccine of paragraph 12, wherein the foreign sequence encoding the one or more avian antigens or immunogens of interest is derived from one or more avian viruses.
19. The immunogenic composition or vaccine of paragraph 12, wherein the foreign sequence encoding the one or more avian antigens or immunogens of interest is derived from avian influenza virus.
20. The immunogenic composition or vaccine of paragraph 19, wherein the foreign sequence encoding the one or more avian antigens or immunogens of interest is selected from the group consisting of genes encoding hemagglutinin, spike protein, other external proteins, nucleoprotein, matrix, neuraminidase and genes coding non-structural proteins such as enzymes and other regulatory proteins.
21. The immunogenic composition or vaccine of paragraph 20, wherein the foreign sequence encoding the one or more avian antigens or immunogens of interest is selected from the group consisting of hemagglutinin subtype 3 and 5.
22. The composition or vaccine of paragraph 12, further comprising an adjuvant.
23. The composition or vaccine of paragraph 12, further comprising an additional vaccine.
24. A method of eliciting an immunogenic response to avian influenza in an avian subject, comprising administering an immunologically effective amount of the composition of any one of paragraphs 12-23 to the avian subject.
25. A method of eliciting an immunogenic response in an avian subject, comprising infecting the avian subject with an immunologically effective amount of an immunogenic composition comprising a recombinant human adenovirus expression vector that comprises and expresses an adenoviral DNA sequence, and a promoter sequence operably linked to a foreign sequence encoding one or more avian antigens or immunogens of interest, wherein the one or more avian antigens or immunogens of interest are expressed at a level sufficient to elicit an immunogenic response to the one or more avian antigens or immunogens of interest in the avian subject, wherein the immunogenic composition is administered via aerosol spray.
26. The method of paragraph 25, wherein the one or more avian antigens or immunogens of interest are derived from avian influenza virus, infectious bursal disease virus, Marek's disease virus, avian herpesvirus, infectious laryngotracheitis virus, avian infectious bronchitis virus, avian reovirus, avipox, fowlpox, canarypox, pigeonpox, quailpox, and dovepox, avian polyomavirus, Newcastle Disease virus, avian pneumovirus, avian rhinotracheitis virus, avian reticuloendotheliosis virus, avian retroviruses, avian endogenous virus, avian erythroblastosis virus, avian hepatitis virus, avian anemia virus, avian enteritis virus, Pacheco's disease virus, avian leukemia virus, avian parvovirus, avian rotavirus, avian leukosis virus, avian musculoaponeurotic fibrosarcoma virus, avian myeloblastosis virus, avian myeloblastosis-associated virus, avian myelocytomatosis virus, avian sarcoma virus, or avian spleen necrosis virus.
27. The method of paragraph 25, wherein the foreign sequence encoding the one or more avian antigens or immunogens of interest is derived from avian influenza virus.
28. The method of paragraph 27, wherein the foreign sequence encoding the one or more avian antigens or immunogens of interest is selected from the group consisting of genes encoding hemagglutinin, spike protein, other external proteins, nucleoprotein, matrix, neuraminidase and genes coding non-structural proteins such as enzymes and other regulatory proteins.
29. The method of paragraph 28, wherein the foreign sequence encoding the one or more avian antigens or immunogens of interest is selected from the group consisting of hemagglutinin subtype 3, 5, 7, and 9.
30. The method of paragraph 25 further comprising an additional vaccine.
31. A method for inoculation of an avian subject, comprising aerosol spray administration of a recombinant human adenovirus containing and expressing an heterologous nucleic acid molecule encoding an antigen of a pathogen of the avian subject.
32. The method of paragraph 31, wherein the human adenovirus comprises sequences derived from adenovirus serotype 5.
33. The method of paragraph 31, wherein the human adenovirus comprises sequences derived from replication-defective adenovirus, non-replicating adenovirus, replication-competent adenovirus, or wild-type adenovirus.
34. The method of paragraph 33, wherein the adenoviral DNA sequence is E1/E3 deleted.
35. The method of paragraph 31, wherein the antigen of a pathogen of the avian is derived from avian influenza virus, infectious bursal disease virus, Marek's disease virus, avian herpesvirus, infectious laryngotracheitis virus, avian infectious bronchitis virus, avian reovirus, avipox, fowlpox, canarypox, pigeonpox, quailpox, and dovepox, avian polyomavirus, Newcastle Disease virus, avian pneumovirus, avian rhinotracheitis virus, avian reticuloendotheliosis virus, avian retroviruses, avian endogenous virus, avian erythroblastosis virus, avian hepatitis virus, avian anemia virus, avian enteritis virus, Pacheco's disease virus, avian leukemia virus, avian parvovirus, avian rotavirus, avian leukosis virus, avian musculoaponeurotic fibrosarcoma virus, avian myeloblastosis virus, avian myeloblastosis-associated virus, avian myelocytomatosis virus, avian sarcoma virus, or avian spleen necrosis virus.
36. The method of paragraph 35, wherein the antigen of a pathogen of the avian is derived from avian influenza virus.
37. The method of paragraph 36, wherein the foreign sequence encoding the one or more avian antigens or immunogens of interest is selected from the group consisting of genes encoding hemagglutinin, spike protein, other external proteins, nucleoprotein, matrix, neuraminidase and genes coding non-structural proteins such as enzymes and other regulatory proteins.
38. The method of paragraph 36, wherein the avian influenza antigens or immunogens are selected from the group consisting of hemagglutinin subtype 3, 5, 7, and 9.
39. The method of paragraph 31, further comprising administering an additional vaccine.
40. An aerosol spray administration apparatus for delivery of an immunogenic composition to one or more avians wherein the apparatus contains a recombinant human adenovirus expression vector expressing one or more avian antigens or immunogens of interest, wherein the apparatus delivers the recombinant human adenovirus to the one or more avians.
41. The apparatus of paragraph 40, wherein the human adenovirus expression vector comprises sequences derived from adenovirus serotype 5.
42. The apparatus of paragraph 40, wherein the human adenovirus expression vector comprises sequences derived from replication-defective adenovirus, non-replicating human adenovirus, replication-competent adenovirus, or wild-type adenovirus.
43. The apparatus of paragraph 40, wherein the adenoviral DNA sequence is E1/E3 deleted.
44. The apparatus of paragraph 40, wherein the one or more avian antigens or immunogens of interest are derived from avian influenza virus, infectious bursal disease virus, Marek's disease virus, avian herpesvirus, infectious laryngotracheitis virus, avian infectious bronchitis virus, avian reovirus, avipox, fowlpox, canarypox, pigeonpox, quailpox, and dovepox, avian polyomavirus, Newcastle Disease virus, avian pneumovirus, avian rhinotracheitis virus, avian reticuloendotheliosis virus, avian retroviruses, avian endogenous virus, avian erythroblastosis virus, avian hepatitis virus, avian anemia virus, avian enteritis virus, Pacheco's disease virus, avian leukemia virus, avian parvovirus, avian rotavirus, avian leukosis virus, avian musculoaponeurotic fibrosarcoma virus, avian myeloblastosis virus, avian myeloblastosis-associated virus, avian myelocytomatosis virus, avian sarcoma virus, or avian spleen necrosis virus.
45. The method of paragraph 40, wherein the one or more avian antigens or immunogens of interest are derived from avian influenza virus.
46. The method of paragraph 40, wherein the foreign sequence encoding the one or more avian antigens or immunogens of interest is selected from the group consisting of genes encoding hemagglutinin, spike protein, other external proteins, nucleoprotein, matrix, neuraminidase and genes coding non-structural proteins such as enzymes and other regulatory proteins.
47. The method of paragraph 40, wherein the avian influenza antigens or immunogens of interest is selected from the group consisting of hemagglutinin subtype 3, 5, 7, and 9.
48. The method of paragraph 40 further comprising administering an additional vaccine.

### REFERENCES

1. Ahmad, I., Kleven, S. H., Avakian, A. P., and Glisson, J. R. (1988) Sensitivity and specificity of Mycoplasma gallisepticum agglutination antigens prepared from medium with artificial liposomes substituting for serum. Avian Dis. 32, 519-26.
2. Al-Mariri, A., Tibor, A., Mertens, P., De Bolle, X., Michel, P., Godefroid, J., Walravens, K., and Letesson, J. J. (2001) Protection of BALB/c mice against Brucella abortus 544 challenge by vaccination with bacterioferritin or P39 recombinant proteins with CpG oligodeoxynucleotides as adjuvant. Infect Immun. 69, 4816-22.
3. Alexander, D. J., Lister, S. A., Johnson, M. J., Randall, C. J., and Thomas, P. J. (1993). An outbreak of highly pathogenic avian influenza in turkeys in Great Britain in 1991. Vet. Rec. 132, 535-536.
4. Alexander D. J. (1990) Avian Paramyxoviridae—recent developments. Vet Microbiol. 23, 103-14.
5. Alexander, D. J., Parsons, G., and Manvell, R. J. (1986) Experimental assessment of the pathogenicity of eight avian influenza A viruses of H5 subtype for 20 chickens, turkeys, ducks and quail. Avian Pathol. 15, 647-662.
6. Ali, H. A., Sawada, T., and Noda K. (2004) Protectivity of an immunoaffinity-purified 39 kDa capsular protein of avian Pasteurella multocida in mice. J. Vet. Med. Sci. 66, 1603-4.
7. Arnold, J. J., Ahsan, F., Meezan, E., and Pillion, D. J. (2004) Correlation of tetradecylmaltoside induced increases in nasal peptide drug delivery with morphological changes in nasal epithelial cells. J. Pharm. Sci. 93, 2205-13.
8. Baert, K., Duchateau, L., De Boever, S., Cherlet, M., and De Backer, P. (2005) Antipyretic effect of oral sodium salicylate after an intravenous E. coli LPS injection in broiler chickens. Br Poult Sci. 46, 137-43.
9. Barbour, E. K., Newman, J. A., Sasipreeyajan, J., Caputa, A. C., and Muneer, M. A. (1989) Identification of the antigenic components of the virulent Mycoplasma gallisepticum (R) in chickens: their role in differentiation from the vaccine strain (F). Vet Immunol, Immunopathol. 21, 197-206.
10. Baserga, R., and Denhardt, D. T. (eds.)(1992), Antisense Strategies, Annals of 40 the New York Academy of Sciences. Vol. 600, New York Academy of Sciences, New York, N.Y.
11. Bashiruddin, J. B., Gould, A. R., and Westbury, H. A. (1992) Molecular pathotyping of two avian influenza viruses isolated during the Victoria 1976 outbreak. Aust. Vet. J. 69, 140-142.
12. Bean, W. J., Cox, N. J., and Kendal, A. P. (1980) Recombination of human influenza A viruses in nature. Nature 284, 638-640.
13. Beard, C. W., Schnitzlein, W. M., and Tripathy, D. N. (1992). Effect of route of administration on the efficacy of a recombinant fowlpox virus against H5N2 avian influenza. Avian Dis 36, 1052-1055.
14. Behr, J. P. (1994) Gene transfer with synthetic cationic amphiphiles: prospects for gene therapy. Bioconjug Chem. 5, 382-9.
15. Belli, S. I., Lee, M., Thebo, P., Wallach, M. G., Schwartsburd, B., and Smith, N. C. (2002) Biochemical characterisation of the 56 and 82 kDa immunodominant gametocyte antigens from Eimeria maxima. Int J. Parasitol. 32, 805-16.
16. Ben Abdelmoumen, B., Roy, R. S., and Brousseau, R. (1999) Cloning of Mycoplasma synoviae genes encoding specific antigens and their use as species-specific DNA probes. J Vet Diagn Invest. 11, 162-9.
17. Bergelson J M, Cunningham J A, Droguett G, Kurt-Jones E A, Krithivas A, Hong J S, et al. Isolation of a common receptor for Coxsackie B viruses and adenoviruses 2 and 5. Science. 1997; 275:1320-1323.
18. Berk, A. J. (1986) Adenovirus promoters and E1A transactivation. Annu Rev Genet. 20, 45-79.
19. Bieth, E., and Darlix, J. L. (1992) Complete nucleotide sequence of a highly infectious avian leukosis virus. Nucleic Acids Res. 20, 367.
20. Bigland, C. H., and Matsumoto, J. J. (1975) Nonspecific reactions to Mycoplasma antigens caused in turkeys sera by Erysipelothrix insidiosa bacterin. Avian Dis. 19, 617-21.
21. Borgan, M. A., Mori, Y., Ito, N., Sugiyama, M., and Minamoto, N. (2003) Antigenic analysis of nonstructural protein (NSP) 4 of group A avian rotavirus strain PO-13. Microbiol Immunol. 47, 661-8.
22. Bovarnick, M. R., Miller, J. C., and Snyder, J. C. (1950) The influence of certain salts, amino acids, sugars, and proteins on the stability of rickettsiae. J. Bacteriol. 59, 509-22.
23. Braathen R, Hohman V S, Brandtzaeg P, Johansen F E. Secretory antibody formation: conserved binding interactions between J chain and polymeric Ig receptor from humans and amphibians. J. Immunol. 2007; 178:1589-97.
24. Brody, S. L., and Crystal, R. G. (1994) Adenovirus-mediated in vivo gene transfer. Ann N Y Acad. Sci. 716, 90-101; discussion 101-3.
25. Brunovskis, P., and Velicer, L. F. (1995) The Marek's disease virus (MDV) unique short region: alphaherpesvirus-homologous, fowlpox virus-homologous, and MDV-specific genes. Virology 206, 324-38.
26. Bunikis, J., Luke, C. J., Bunikiene, E., Bergstrom, S., and Barbour, A. G. (1998) A surface-exposed region of a novel outer membrane protein (P66) of Borrelia spp. is variable in size and sequence. J. Bacteriol. 180, 1618-23.
27. Cao, Y. C., Yeung, W. S., Law, M., Bi, Y. Z., Leung, F. C., and Lim, B. L. (1998) Molecular characterization of seven Chinese isolates of infectious bursal disease virus: classical, very virulent, and variant strains Avian Dis. 42, 340-51.
28. Casais, R., Dove, B., Cavanagh, D., and Britton, P. (2003) Recombinant avian infectious bronchitis virus expressing a heterologous spike gene demonstrates that the spike protein is a determinant of cell tropism. J. Virol. 77, 9084-9.
29. Chambers, T. M., Kawaoka, Y., and Webster, R. G. (1988). Protection of 10 chickens from lethal influenza infection by vaccinia-expressed hemagglutinin. Virology 167, 414-421.
30. Chieffi G, Baccari G C, Di Matteo L, d'Istria M, Minucci S, Varriale B. Cell biology of the harderian gland. Int. Rev. Cytol. 1996; 168:1-80.
31. Chiocca, S., Kurzbauer, R., Schaffner, G., Baker, A., Mautner, V., and Cotten, M. (1996). The complete DNA sequence and genomic organization of the avian adenovirus CELO. J Virol 70, 2939-2949.
32. Claas, E. J., Osterhaus, A. E., Van Beek, R., De Jong, J. C., Rimmelzwaan, G. F., Senne, D. A., Krauss, S., Shortridge, K. F., and Webster, R. G. (1998) Human influenza A H5N1 virus related to a highly pathogenic avian influenza virus Lancet 351, 472-477.
33. Constantinoiu, C. C., Lillehoj, H. S., Matsubayashi, M., Tani, H., Matsuda, H., Sasai, K., Baba, E. (2004) Characterization of stage-specific and cross-reactive antigens from Eimeria acervulina by chicken monoclonal antibodies. J Vet Med. Sci. 66, 403-8.
34. Cook, J. K. (2000) Avian rhinotracheitis. Rev Sci Tech. 19, 602-13.
35. Crawford, J., Wilkinson, B., Vosnesensky, A., Smith, G., Garcia, M., Stone, H., and Perdue, M. L. (1999) Baculovirus-derived hemagglutinin vaccines protect against lethal influenza infections by avian H5 and H7 subtypes. Vaccine 17, 2265-2274.
36. Crawford, J. M., Garcia, M., Stone, H., Swayne, D., Slemons, R., and Perdue, M. L. (1998). Molecular characterization of the hemagglutinin gene and oral immunization with a waterfowl-origin avian influenza virus. Avian Dis 42, 486-496.
37. Crook, S, and Lebleu, B. (eds.)(1993) Antisense Research and Applications, CRC Press, Boca Raton, Fla.
38. Cross, G. M. (1987) The status of avian influenza in poultry in Australia, p. 96-103. In Proceedings of the Second International Symposium on Avian Influenza.
39. Coussens, P. M., and Velicer, L. F. (1988) Structure and complete nucleotide sequence of the Marek's disease herpesvirus gp57-65 gene. J. Virol. 62, 2373-9.
40. Cova, L., Duflot, A., Prave, M., and Trepo, C. (1993) Duck hepatitis B virus infection, aflatoxin B1 and liver cancer in ducks. Arch Virol Suppl. 8, 81-7.
41. Curiel, D. T. (1994). High-efficiency gene transfer employing adenovirus-polylysine-DNA complexes. Nat Immun 13, 141-164.
42. Czerkinsky C, Nilsson L A, Nygren H, Ouchterlony O, Tarkowski A. A solid-phase enzyme-linked immunospot (ELISPOT) assay for enumeration of specific antibody-secreting cells. J. Immunol. Methods 1983; 65:109-121.
43. Davison E, Kirby I, Whitehouse J, Hart I, Marshall J F, Santis G. Adenovirus type 5 uptake by lung adenocarcinoma cells in culture correlates with Ad5 fibre binding is mediated by alpha(v)beta1 integrin and can be modulated by changes in beta1 integrin function. J. Gene Med. 2001; 3:550-559.
44. de Gruijl T D, Ophorst O J, Goudsmit J, Verhaagh S, Lougheed S M, Radosevic K, et al. Intradermal delivery of adenoviral type-35 vectors leads to high efficiency transduction of mature, CD8+T cell-stimulating skin-emigrated dendritic cells. J. Immunol. 2006; 177:2208-2215.
45. Ding, X., Lillehoj, H. S., Quiroz, M. A., Bevensee, E., and Lillehoj, E. P. (2004) Protective immunity against Eimeria acervulina following in ovo immunization with a recombinant subunit vaccine and cytokine genes. Infect Immun. 72, 6939-44.
46. Djeraba, A., Musset, E., Lowenthal, J. W., Boyle, D. B., Chausse, A. M., Peloille, M., and Quere, P. (2002) Protective effect of avian myelomonocytic growth factor in infection with Marek's disease virus. J. Virol. 76, 1062-70.
47. Dormitorio, T. V., Giambrone, J. J., and Duck, L. W. (1997) Sequence comparisons of the variable VP2 region of eight infectious bursal disease virus isolates. Avian Dis. 41, 36-44.
48. Dornburg, R. (1995) Reticuloendotheliosis viruses and derived vectors. Gene Ther. 2, 301-10.
49. Easterday, B. C., Hinshaw, V. S., and Halvorson, D. A. (1997) Influenza, p. 583-605. In B. W. Calnek, H. J. Barnes, C. W. Beard, L. R. McDougald, and Y. M. Saif (eds), Diseases of Poultry. Iowa State University Press, Ames.
50. Eckroade, R. J. and Bachin, L. A. S. (1987) Avian influenza in Pennsylvania: the beginning, p. 22-32. In Proceedings of the Second International Symposium on Avian Influenza.
51. Eckstein, F. (eds.) (1992) Oligonucleotides and Analogues, A Practical Approach, Oxford University Press, New York, N.Y.
52. Evans, R. K., Nawrocki, K. K., Isopi, L. A., Williams, D. M., Casimiro, D. R., Chin, S., Chen, M., Zhu, D. M., Shiver, J. W., Volkin, D. B. (2004) Development of stable liquid formulations for adenovirus-based vaccines. J. Pharm. Sci. 93, 2458-2475.
53. Fields, B. N., Howley, P. M., Griffin, D. E., Lamb, R. A., Martin, M. A., Roizman, B., Straus, S. E., and Knipe, D. M. (eds)(2001) Fields—Virology, Lippincott, Williams, and Wilkins, Philadelphia, Pa.
54. Francois, A., Chevalier, C., Delmas, B., Eterradossi, N., Toquin, D., Rivallan, G., and Langlois, P. (2004). Avian adenovirus CELO recombinants expressing VP2 of infectious bursal disease virus induce protection against bursal disease in chickens. Vaccine 22, 2351-2360.
55. Fynan, E. F., Webster, R. G., Fuller, D. H., Haynes, J. R., Santoro, J. C., and Robinson, H. L. (1993). DNA vaccines: Protective immunizations by parenteral, mucosal, and gene-gun inoculations. Proc Natl Acad Sci USA 90, 11478-11482.
56. Gao, W., Soloff, A. C. and Lu, X. et al., (2006) Protection of mice and poultry from lethal H5N1 avian influenza virus through adenovirus-based immunization. J. Virol. 80, 1959.
57. Garcia, M., Crawford, J. M., Latimer, J. W., Rivera-Cruz, E., and Perdue, M. L. (1996) Heterogeneity in the haemagglutinin gene and emergence of the highly pathogenic phenotype among recent H5N2 avian influenza viruses from Mexico. J. Gen Virol. 77, 1493-1504.
58. Garcia, A. Johnson, H., Srivastava, D. K., Jayawardene, D. A., Wehr, D. R., Webster, R. G., (1998) Efficacy of inactivated H5N2 influenza vaccines against lethal A/Chicken/Queretaro/19/95 infection. Avian Dis. 42, 248.
59. Garcia-Sastre, A., Egorov, A., Matassov, D., Brandt, S., Levy, D. E., Durbin, J. E., Palese, P., and Muster, T. (1998) Influenza A virus lacking the NS1 gene replicates in interferon-deficient systems. Virology 252, 324-330.
60. Gildersleeve, R. P., (1993) In ovo technology update. Zootec. Int. 73-77.
61. Gildersleeve, R. P., Hoyle, C. M., Miles, A. M., Murray, D. L., Ricks, C. A., Secrest, M. N., Williams, C. J., and Womack, C. L. (1993) Developmental performance of an egg injection machine for administration of Marek's disease vaccine. J. Appl. Poult. Res. 2, 337-346.
62. Gorman, L., Suter, D., Emerick, V., Schumperli, D., and Kole, R. (1998) Stable alteration of pre-mRNA splicing patterns by modified U7 small nuclear RNAs. Proc Natl Acad Sci USA. 95, 4929-34.
63. Gottschalk, A. (1957) The specific enzyme of influenza virus and Vibrio cholerae. Biochim. Biophys. Acta 23, 645-646.
64. Graf, T., and Beug, H. (1978) Avian leukemia viruses: interaction with their 40 target cells in vivo and in vitro. Biochim Biophys Acta 516, 269-99.
65. Graf, T., and Beug, H. (1983) Role of the v-erbA and v-erbB oncogenes of avian erythroblastosis virus in erythroid cell transformation. Cell. 34, 7-9.
66. Graham, F. L., and Prevec, L. (1995). Methods for construction of adenovirus vectors. Mol Biotechnol 3, 207-220.
67. Grim, K. C., McCutchan, T., Li, J., Sullivan, M., Graczyk, T. K., McConkey, G., and Cranfield, M. (2004) Preliminary results of an anticircumsporozoite DNA vaccine trial for protection against avian malaria in captive African black-footed penguins (Spheniscus demersus). J Zoo Wildl Med. 35, 154-61.
68. Guo, Z. S., Wang, L. H., Eisensmith, R. C., and Woo, S. L. (1996) Evaluation of promoter strength for hepatic gene expression in vivo following adenovirus-mediated gene transfer. Gene Ther. 3, 802-10.
69. Hansell C, Zhu X W, Brooks H, Sheppard M, Withanage S, Maskell D, et al. Unique features and distribution of the chicken CD83+ cell. J. Immunol. 2007; 179:5117-5125.
70. Havenga, M. J., Lemckert, A. A., Grimbergen, J. M., Vogels, R., Huisman, L. G., Valerio, D., Bout, A., and Quax, P. H. (2001) Improved adenovirus vectors for infection of cardiovascular tissues. J. Virol. 75, 3335-42.
71. Havenga, M. J., Lemckert, A. A., Ophorst, O. J., van Meijer, M., Germeraad, W. T., Grimbergen, J., van Den Doel, M. A., Vogels, R., van Deutekom, J., Janson, A. A., de Bruijn, J. D., Uytdehaag, F., Quax, P. H., Logtenberg, T., Mehtali, M., and Bout, A. (2002) Exploiting the natural diversity in adenovirus tropism for therapy and prevention of disease. J. Virol. 76, 4612-20.
72. He, T. C., Zhou, S., da Costa, L. T., Yu, J., Kinzler, K. W., Vogelstein, B. (1998) A simplified system for generating recombinant adenovirus. Proc. Nat. Acad. Sci. USA. 95, 2509 (1998).
73. He, T. C., Zhou, S., da Costa, L. T., Yu, J., Kinzler, K. W., and Vogelstein, B. 25 (1998). A simplified system for generating recombinant adenoviruses. Proc Natl Acad Sci USA 95, 2509-2514.
74. Heine, H. G., Haritou, M., Failla, P., Fahey, K., and Azad, A. (1991) Sequence analysis and expression of the host-protective immunogen VP2 of a variant strain of infectious bursal disease virus which can circumvent vaccination with standard type I strains. J Gen Virol. 72, 1835-43.
75. Higgins, D. A., Henry, R. R., and Kounev, Z. V. (2000) Duck immune responses to Riemerella anatipestifer vaccines. Dev Comp Immunol. 24, 153-67.
76. Hilleman, M. R. (2002). Realities and enigmas of human viral influenza: pathogenesis, epidemiology and control. Vaccine 20, 3068-3087.
77. Hinshaw, V. S., Bean, W. J., Webster, R. G., and Sriram, G. (1980) Genetic reassortment of influenza A viruses in the intestinal tract of ducks. Virology 102, 412-419.
78. Hilton, L. S., Bean, A. G., Kimpton, W. G., and Lowenthal, J. W. (2002) Interleukin-2 directly induces activation and proliferation of chicken T cells in vivo. J Interferon Cytokine Res. 22, 755-63.
79. Hirst, G. K. (1941) Agglutination of red cells by allantoic fluid of chick embryos infected with influenza virus. Science 94, 22-23.
80. Hong S S, Karayan L, Tournier J, Curiel D T, Boulanger P A. Adenovirus type 5 fiber knob binds to MHC class I α2 domain at the surface of human epithelial and B-lymphoblastoid cells. EMBO J. 1997; 16:2294-2306.
81. Horimoto, T., Rivera, E., Pearson, J., Senne, D., Krauss, S., Kawaoka, Y., and Webster, R. G. (1995) Origin and molecular changes associated with emergence of a highly pathogenic H5N2 influenza virus in Mexico. Virology 213, 223-230.
82. Israeli, E., Shaffer, B. T., and Lighthart, B. (1993) Protection of freeze-dried Escherichia coli by trehalose upon exposure to environmental conditions. Cryobiology 30, 519-23.
83. Ito A. Gotanda, T., Kobayashi, S., Kume, K., Sugimoto, C., and Matsumura, T. (2005) Increase of antibody titer against Leucocytozoon caulleryi by oral administration of recombinant R7 antigen. J. Vet. Med. Sci. 67, 211-3.
84. Jan, G., Le Henaff, M., Fontenelle, C., and Wroblewski, H. (2001) Biochemical and antigenic characterisation of Mycoplasma gallisepticum membrane proteins P52 and P67 (pMGA). Arch Microbiol. 177, 81-90.
85. Jochemsen, A. G., Peltenburg, L. T., to Pas, M. F., de Wit, C. M., Bos, J. L., and van der Eb, A. J. (1987) Activation of adenovirus 5 E1A transcription by region E1B in transformed primary rat cells. EMBO J. 6, 3399-405.
86. Johansen F E, Braathen R, Brandtzaag P. Role of J chain in secretory immunoglobulin formation. Scand. J. Immunol. 2000; 52:240-248.
87. Johnson, D. C., Maxfield, B. G., and Moulthrop, J. I. (1976) Epidemiologic studies of the 1975 avian influenza outbreak in chickens in Alabama. Avian Dis. 21, 167-177.
88. Johnston, P. A., Liu, H., O'Connell, T., Phelps, P., Bland, M., Tyczkowski, J., Kemper, A., Harding, T., Avakian, A., Haddad, E., et al. (1997). Applications in in ovo technology. Poult Sci 76, 165-178.
89. Joliot, V., Boroughs, K., Lasserre, F., Crochet, J., Dambrine, G., Smith, R. E., and Perbal, B. (1993) Pathogenic potential of myeloblastosis-associated virus: implication of env proteins for osteopetrosis induction. Virology 195, 812-9.
90. Kaleta, E. F. (1990) Herpesviruses of birds—a review. Avian Pathol. 10, 193-211.
91. Kapczynski, D. R., Hilt, D. A., Shapiro, D., Sellers, H. S., and Jackwood, M. W. (2003). Protection of chickens from infectious bronchitis by in ovo and intramuscular vaccination with a DNA vaccine expressing the S1 glycoprotein. Avian Dis 47, 272-285.
92. Karaca, K., Sharma, J. M., Winslow, B. J., Junker, D. E., Reddy, S., Cochran, M., and McMillen, J. (1998) Recombinant fowlpox viruses coexpressing chicken type I IFN and Newcastle disease virus HN and F genes: influence of IFN on protective efficacy and humoral responses of chickens following in ovo or post-hatch administration of recombinant viruses. Vaccine. 16, 1496-503.
93. Karim, M. J., Basak, S.C., and Trees, A. J. (1996) Characterization and immunoprotective properties of a monoclonal antibody against the major oocyst wall protein of Eimeria tenella. Infect Immun. 64, 1227-32.
94. Kariyawasam, S., Wilkie, B. N., Hunter, D. B., and Gyles, C. L. (2002) Systemic and mucosal antibody responses to selected cell surface antigens of avian pathogenic Escherichia coli in experimentally infected chickens. Avian Dis. 46, 668-78.
95. Kasten, R. W., Hansen, L. M., Hinojoza, J., Bieber, D., Ruehl, W. W., and Hirsh, D. C. (1995) Pasteurella multocida produces a protein with homology to the P6 outer membrane protein of Haemophilus influenzae. Infect Immun. 63, 989-93.
96. Kawai, S., Goto, N., Kataoka, K., Saegusa, T., Shinno-Kohno, H., and Nishizawa, M. (1992) Isolation of the avian transforming retrovirus, AS42, carrying the v-maf oncogene and initial characterization of its gene product. Virology 188, 778-84.
97. Kawaoka, Y., Nestorowicz, A., Alexander, D. J., and Webster, R. G. (1987) Molecular analyses of the hemagglutinin genes of H5 influenza viruses: origin of a virulent turkey strain. Virology 158, 218-227.
98. Kawaoka, Y., Krauss, S., and Webster, R. G. (1989). Avian-to-human transmission of the PB1 gene of influenza A viruses in the 1957 and 1968 pandemics. J Virol 63, 4603-4608.
99. Kida, H., Yanagawa, R., and Matsuoka, Y. (1980) Duck influenza lacking evidence of disease signs and immune response. Infect. Immun. 30, 547-553.
100. Kobayashi K, Hirai H. Studies on subunit components of chicken polymeric immunoglobulins. J. Immunol. 1980; 124:1695-1704.
101. Kobayashi, Y., Horimoto, T., Kawaoka, Y., Alexander, D. J., and Itakura, C. (1996) Pathological studies of chickens experimentally infected with two highly pathogenic avian influenza strains. Avian Pathol. 25, 285-304.
102. Konz, J. O. et al. (2005) Serotype specificity of adenovirus purification using anion-exchange chromatography. Hum. Gene Ther. 16, 1346-1353.
103. Kozak, M. (1986). Point mutations define a sequence flanking the AUG initiator codon that modulates translation by eukaryotic ribosomes. Cell 44, 283-292.
104. Lamb, R. A. and Krug, R. M. (1996) Orthomyxoviruses: the viruses and their replication, p. 1353-1395. In B. N. Fields, D. M. Knipe, and P. M. Howley (ed.), Fields virology, 3^{rd} ed. Lippincott-Raven, Philadelphia, Pa.
105. Langer, R. C., Li, F., and Vinetz, J. M. Identification of novel Plasmodium gallinaceum zygote- and ookinete-expressed proteins as targets for blocking malaria transmission. Infect Immun. 70, 102-6.
106. Lee, C. W., Senne, D. A., and Suarez, D. L. (2004). Generation of reassortant influenza vaccines by reverse genetics that allows utilization of a DIVA (Differentiating Infected from Vaccinated Animals) strategy for the control of avian influenza. Vaccine 22, 3175-3181.
107. Lewis, J. A., Brown, E. L. & Duncan, P. A. (2006) Approaches to the release of a master cell bank of PER.C6 cells; a novel cell substrate for the manufacture of human vaccines. Dev. Biol. (Basel) 123, 165-176.
108. Li, W., Watarai, S., Iwasaki, T., and Kodama, H. (2004) Suppression of Salmonella enterica serovar Enteritidis excretion by intraocular vaccination with fimbriae proteins incorporated in liposomes. Dev Comp Immunol. 28, 29-38.
109. Lillehoj, H. S., Ding, X., Quiroz, M. A., Bevensee, E., and Lillehoj, E. P. (2005) Resistance to intestinal coccidiosis following DNA immunization with the cloned 3-1E Eimeria gene plus IL-2, IL-15, and IFN-gamma. Avian Dis. 49, 112-7.
110. Mansikka A. Chicken IgA H chains Implications concerning the evolution of H chain genes. J. Immunol. 1992; 149:855-861.
111. Marconi, R. T., Samuels, D. S., Schwan, T. G., and Garon, C. F. (1993) Identification of a protein in several Borrelia species which is related to OspC of the Lyme disease spirochetes. J Clin Microbiol. 31, 2577-83.
112. Mata, J. E., Joshi, S. S., Palen, B., Pirruccello, S. J., Jackson, J. D., Elias, N., Page, T. J., Medlin, K. L., and Iversen, P. L. (1997) A hexameric phosphorothioate oligonucleotide telomerase inhibitor arrests growth of Burkitt's lymphoma cells in vitro and in vivo. Toxicol Appl Pharmacol. 144, 189-97.
113. McEwan, N. R., and Gatherer, D. (1998) Adaptation of standard spreadsheet software for the analysis of DNA sequences. Biotechniques 24, 131-6, 138.
114. Milligan, J. F., Matteucci, M. D., and Martin, J. C. (1993) Current concepts in antisense drug design. J Med. Chem. 36, 1923-37.
115. Mills, C. K., and Gherna, R. L. (1988) Cryopreservation studies of Campylobacter. Cryobiology 25, 148-52.
116. Mo, I. P., Brugh, M., Fletcher, O. J., Rowland, G. N., and Swayne, D. E. (1997) Comparative pathology of chickens experimentally inoculated with avian influenza viruses of low and high pathogenicity. Avian Dis. 41, 125-136.
117. Mori, Y., Borgan, M. A., Takayama, M., Ito, N., Sugiyama, M., and Minamoto, N. (2003) Roles of outer capsid proteins as determinants of pathogenicity and host range restriction of avian rotaviruses in a suckling mouse model. Virology 316, 126-34.
118. Molinier-Frenkel, V., Lengagne, R., Gaden, F., Hong, S. S., Choppin, J., Gahery-Segard, H., Boulanger, P., and Guillet, J. G. (2002). Adenovirus hexon protein is a potent adjuvant for activation of a cellular immune response. J Virol 76, 127-135.
119. Murphy, B. R. and Webster, R. G. (1996) Orthomyxoviruses, p. 1397-1445. In B. N. Fields, D. M. Knipe, and P. M. Howley (ed.), Fields virology, 3^{rd} ed. Lippincott-Raven, Philadelphia, Pa.
120. Nakamura, Y., Wada, K., Wada, Y., Doi, H., Kanaya, S., Gojobori, T., and Ikemura, T. (1996) Codon usage tabulated from the international DNA sequence databases. Nucleic Acids Res. 24, 214-5.
121. Neckameyer, W. S., and Wang, L. H. (1985) Nucleotide sequence of avian sarcoma virus UR2 and comparison of its transforming gene with other members of the tyrosine protein kinase oncogene family. J. Virol. 53, 879-84.
122. Nestorowicz, A., Kawaoka, Y., Bean, W. J., and Webster, R. G., (1987) Molecular analysis of the hemagglutinin genes of Australian H7N7 influenza viruses: role of passerine birds in maintenance or transmission? Virology 160, 411-418.
123. Noormohammadi, A. H., Browning, G. F., Cowling, P. J., O'Rourke, D., Whithear, K. G., and Markham, P. F. (2002a) Detection of antibodies to Mycoplasma gallisepticum vaccine ts-11 by an autologous pMGA enzyme-linked immunosorbent assay. Avian Dis. 46, 405-11.
124. Noormohammadi, A. H., Browning, G. F., Jones, J., and Whithear, K. G. (2002b) Improved detection of antibodies to Mycoplasma synoviae vaccine MS-H using an autologous recombinant MSPB enzyme-linked immunosorbent assay. Avian Pathol. 31, 611-7.
125. Normile, D. (2004). Influenza: girding for disaster. Vaccinating birds may help to curtail virus's spread. Science 306, 398-399.
126. O'Neill, R. E., Talon, J., and Palese, P. (1998) The influenza virus NEP (NS2 protein) mediates the nuclear export of viral ribonucleoproteins. EMBO J. 17, 288-296.
127. Ochoa-Reparaz, J., Sesma, B., Alvarez, M., Jesus Renedo, M., Irache, J. M., and Gamazo, C. (2004) Humoral immune response in hens naturally infected with Salmonella Enteritidis against outer membrane proteins and other surface structural antigens. Vet Res. 35, 291-8.
128. Okada N, Tsukada Y, Nakagawa S, Mizuguchi H, Mori K, Saito T, et al. Efficient gene delivery into dendritic cells by fiber-mutant adenovirus vectors. Biochem. Biophys. Res. Commun. 2001; 282: 173-179.
129. Oshop, G. L., Elankumaran, S., and Heckert, R. A. (2002). DNA vaccination in the avian. Vet Immunol Immunopathol 89, 1-12.
130. Oshop, G. L., Elankumaran, S., Vakharia, V. N., and Heckert, R. A. (2003). In ovo delivery of DNA to the avian embryo. Vaccine 21, 1275-1281.
131. Paulson, J. C. (1985) Interactions of animal viruses with cell surface receptors, p. 131-219. In M. Connor (ed.), The receptors. Academic Press, Inc., Orlando, Fla.
132. Payne A P. The harderian gland: a tercentennial review. J. Anat. 1994; 185:1-49.
133. Pei J, Collisson E W. Specific antibody secreting cells from chickens can be detected by three days and memory B cells by three weeks post-infection with the avian respiratory coronavirus. Dev. Comp. Immunol. 2005; 29:153-160.
134. Perbal, B. (1995) Pathogenic potential of myeloblastosis-associated viruses. Infect Agents Dis. 4, 212-27.
135. Perdue, M. L., Garcia, M., and Senne, D. (1997) Virulence-associated sequence duplication at the hemagglutinin cleavage site of avian influenza viruses. Virus Res. 49, 173-186.
136. Petropoulos, C. J., Appendix 2: Retroviral Taxonomy, protein structure, sequences, and genetic maps. In: Coffin, J. M. (Ed.); RETROVIRUSES: Cold Spring Harbor Laboratory Press, Cold Spring Harbor, N.Y. USA (1997).
137. Pinto, L. H., Holsinger, L. J., and Lamb, R. A. (1992) Influenza virus M2 protein has ion channel activity. Cell 69, 517-528.
138. Pitcovski, J., Mualem, M., Rei-Koren, Z., Krispel, S., Shmueli, E., Peretz, Y., Gutter, B., Gallili, G. E., Michael, A., and Goldberg, D. (1998) The complete DNA sequence and genome organization of the avian adenovirus, hemorrhagic enteritis virus. Virology 249, 7-15.
139. Pogonka, T., Klotz, C., Kovacs, F., and Lucius, R. (2003) A single dose of recombinant Salmonella typhimurium induces specific humoral immune responses against heterologous Eimeria tenella antigens in chicken. Int J. Parasitol. 33, 81-8.
140. Purchase, H. G., and Witter, R. L. (1975) The reticuloendotheliosis viruses. Curr Top Microbiol Immunol. 71, 103-24.
141. Rajakumar, A., Swierkosz, E. M., and Schulze, I. T. (1990). Sequence of an influenza virus hemagglutinin determined directly from a clinical sample. Proc Natl Acad Sci USA 87, 4154-4158.
142. Randall T D, King L B, Corley R B. The biological effects of IgM hexamer formation. Eur. J. Immunol. 1990; 20:1971-1979.
143. Rea D, Schagen F H E, Hoeben R C, Mehtali M, Havenga M J E, Toes R E M, et al. Adenoviruses activate human dendritic cells without polarization toward a T-helper type 1-inducing subset. J. Virol. 1999; 73:10245-10253.
144. Regelson, W., Kuhar, S., Tunis, M., Fields, J., Johnson, J., Gluesenkamp, E. (1960) Synthetic polyelectrolytes as tumour inhibitors. Nature. 186, 778-80.
145. Richardson, J. C., and Akkina, R. K. (1991) NS2 protein of influenza virus is found in purified virus and phosphorylated in infected cells. Arch. Virol. 116, 69-80.
146. Rimler, R. B. (2001) Purification of a cross-protective antigen from Pasteurella multocida grown in vitro and in vivo. Avian Dis. 45, 572-80.
147. Roberts, B. E., Miller, J S, Kimelman, D., Cepko, C. L., Lemischka, I. R., and Mulligan, R. C. (1985) Individual adenovirus type 5 early region 1A gene products elicit distinct alterations of cellular morphology and gene expression. J. Virol. 56, 404-13.
148. Roelvink P W, Lizonova A, Lee J G M, Li Y, Bergelson J M, Finberg R W, et al. The Coxsackievirus-Adenovirus Receptor Protein Can Function as a Cellular Attachment Protein for Adenovirus Serotypes from Subgroups A, C, D, E, and F. J. Virol. 1998; 72: 7909-7915.
149. Röhm, C., Siiss, J., Pohle, V., and Webster, R. G. (1996a) Different hemagglutinin cleavage site variants of H7N7 in an influenza outbreak in chickens in Leipzig, Germany. Virology 218, 253-257.
150. Röhm, C., Zhou, N. A., Siiss, J., Mackenzie, J., and Webster, R. G. (1996b) Characterization of a novel influenza hemagglutinin, H 15: criteria for determination of influenza A subtypes. Virology 217, 508-516.
151. Roland, K., Karaca, K., and Sizemore, D. (2004) Expression of Escherichia coli antigens in Salmonella typhimurium as a vaccine to prevent airsacculitis in chickens. Avian Dis. 48, 595-605.
152. Rosenberger, J. K., and Cloud, S. S. (1998) Chicken anemia virus. Poult Sci. 77, 1190-2.
153. Ross, L. J., Sanderson, M., Scott, S. D., Binns, M. M., Doel, T., and Milne, B. (1989) Nucleotide sequence and characterization of the Marek's disease virus homologue of glycoprotein B of herpes simplex virus. J Gen Virol. 70, 1789-804.
154. Ross, L. J., and Binns, M. M. (1991) Properties and evolutionary relationships of the Marek's disease virus homologues of protein kinase, glycoprotein D and glycoprotein I of herpes simplex virus. J Gen Virol. 72, 939-47.
155. Rott, O., Kroger, M., Muller, H., and Hobom, G. (1988) The genome of budgerigar fledgling disease virus, an avian polyomavirus. Virology 165, 74-86.
156. Rovigatti, U. G., and Astrin, S. M. (1983) Avian endogenous viral genes. Curr Top Microbiol Immunol. 103, 1-21.
157. Saito, S., Fujisawa, A., Ohkawa, S., Nishimura, N., Abe, T., Kodama, K., Kamogawa, K., Aoyama, S., Iritani, Y., and Hayashi, Y. (1993) Cloning and DNA sequence of a 29 kilodalton polypeptide gene of Mycoplasma gallisepticum as a possible protective antigen. Vaccine 11, 1061-6.
158. Sambri, V., Marangoni, A., Olmo, A., Storni, E., Montagnani, M., Fabbi, M., and Cevenini, R. (1999) Specific antibodies reactive with the 22-kilodalton major outer surface protein of Borrelia anserina Ni-NL protect chicks from infection. Infect Immun. 67, 2633-7.
159. Sambrook, J., Russell, D. W., and Sambrook, J. (2001) Molecular Cloning, Cold Spring Harbor Press, Cold Spring Harbor, N.Y.
160. Samstag, W., Eisenhardt, S., Offensperger, W. B., and Engels, J. W. (1996) Synthesis and properties of new antisense oligodeoxynucleotides containing benzylphosphonate linkages. Antisense Nucleic Acid Drug Dev. 6, 153-6.
161. Sapinoro R, Maguire C A, Burgess A, Dewhurst S. Enhanced transduction of dendritic cells by FcgammaR1-targeted adenovirus vectors. J. Gene Med. 2007; 9:1033-45.
162. Schaap, D., Arts, G., Kroeze, J., Niessen, R., Roosmalen-Vos, S. V., Spreeuwenberg, K., Kuiper, C. M., Beek-Verhoeven, N. V., Kok, J. J., Knegtel, R. M., and Vermeulen, A. N. (2004) An Eimeria vaccine candidate appears to be lactate dehydrogenase; characterization and comparative analysis. Parasitology. 128, 603-16.
163. Schijns, V. E., Weining, K. C., Nuijten, P., Rijke, E. O., and Staeheli, P. (2000) Immunoadjuvant activities of E. coli- and plasmid-expressed recombinant chicken IFN-alpha/beta, IFN-gamma and IL-lbeta in 1-day- and 3-week-old chickens. Vaccine. 18, 2147-54.
164. Schultz-Cherry, S., Dybing, J. K., Davis, N. L., Williamson, C., Suarez, D. L., Johnston, R., and Perdue, M. L. (2000). Influenza virus (A/HK/156/97) hemagglutinin expressed by an alphavirus replicon system protects chickens against lethal infection with Hong Kong-origin H5N1 viruses. Virology 278, 55-59.
165. Seal, B. S. (2000) Avian pneumoviruses and emergence of a new type in the United States of America. Anim Health Res Rev. 1, 67-72.
166. Senne, D. A., in A laboratory manual for the isolation and identification of avian pathogens Swayne, D. E., Glisson, J. R., Jackwood, M. W., Pearson, J. E., Reed, W. M., Eds. (American Association of Avian Pathologists, Kennett Square, Pa., 1998) pp. 235-240.
167. Sharma, J. M. (1985) Embryo vaccination with infectious bursal disease virus alone or in combination with Marek's disease vaccine. Avian Dis. 27, 134-139.
168. Sharma, J. M. and Burmester, B. R. (1982) Resistance of Marek's disease at hatching in chickens vaccinated as embryos with the turkey herpesvirus. Avian Dis. 26, 134-139.
169. Shi, Z., Zeng, M., Yang, G., Siegel, F., Cain, L. J., van Kampen, K., Elmets, C. A., and Tang, D. C. Protection against tetanus by needle-free inoculation of adenovirus-vectored nasal and epicutaneous vaccines. J. Virol. 75, 11474 (2001).
170. Song W, Vaerman J-P, Mostov K E. Dimeric and tetrameric IgA are transcytosed equally by the polymeric Ig receptor. J. Immunol. 1995; 155: 715-721.
171. Spackman, E, Senne, D. A., Myers, T. J., Bulaga, L. L., Garber, L. P., Perdue, M. L., Lohman, K., Daum, L. T., Suarez, D. L. Development of a real-time reverse transcriptase PCR assay for type A influenza virus and the avian H5 and H7 hemagglutinin subtypes. J. Clin. Microbiol. 40, 3256 (2002).
172. Spandidos, D. A., and Graham, A. F. (1976) Physical and chemical characterization of an avian reovirus. J. Virol. 19, 968-76.
173. Strauss-Soukup, J. K., Vaghefi, M. M., Hogrefe, R. I., Maher, L. J., 3rd. (1997) Effects of neutralization pattern and stereochemistry on DNA bending by methylphosphonate substitutions. Biochemistry. 36, 8692-8.
174. Suarez D. L., Perdue, M. K., Cox, N., Rowe, T., Bender, C., Huang, J., and Swayne, D. E. (1998) Comparisons of highly virulent H5N1 influenza A viruses isolated from humans and chickens from Hong Kong. J. Virol. 72, 6678-6688.
175. Subbarao, K., Klimov, A., Katz, J., Regnery, H., Lim, W., Hall, H., Perdue, M., Swayne, D., Bender, C., Huang, J., et al. (1998). Characterization of an avian influenza A (H5N1) virus isolated from a child with a fatal respiratory illness. Science 279, 393-396.
176. Swayne, D. E., Perdue, M. L., Garcia, M., Rivera-Cruz, E., and Brugh, M. (1997) Pathogenicity and diagnosis of H5N2 Mexican avian influenza viruses in chickens. Avian Dis. 41, 335-346.
177. Swayne, D. E. (2003). Vaccines for List A poultry diseases: emphasis on avian influenza. Dev Biol (Basel) 114, 201-212.
178. Swayne, D. E., Senne, D. A. and Beard., C. W., in A laboratory Manual for the Isolation and Identification of Avian Pathogens D. E. Swayne, J. R. Glisson, M. W. Jackwood, J. E. Pearson, and W. M. Reed, Eds. (American Association of Avian Pathologists, Kennett Square, Pa., 1998) pp. 150-155.
179. Tajima, O., Onaga, H., and Nakamura, T. (2003) An enzyme-linked immunosorbent assay with the recombinant merozoite protein as antigen for detection of antibodies to Eimeria necatrix. Avian Dis. 47, 309-18.
180. Takahashi T, Iwase T, Tachibana T, Komiyama K, Kobayashi K, Chen C L, et al. Cloning and expression of the chicken immunoglobulin joining (J)-chain cDNA. Immunogenetics. 2000; 51:85-91.
181. Tan, P. K., Michou, A. I., Bergelson, J. M., and Cotten, M. (2001). Defining CAR as a cellular receptor for the avian adenovirus CELO using a genetic analysis of the two viral fibre proteins. J Gen Virol 82, 1465-1472.
182. Telling, G. C., Perera, S., Szatkowski-Ozers, M., and Williams, J. (1994) Absence of an essential regulatory influence of the adenovirus E1B 19-kilodalton protein on viral growth and early gene expression in human diploid WI38, HeLa, and A549 cells. J. Virol. 68, 541-7.
183. Thayer, S. G. and Beard, C. W., in A laboratory Manual for the Isolation and Identification of avian pathogens D. E. Swayne, J. R. Glisson, M. W. Jackwood, J. E. Pearson, and W. M. Reed, Eds. (American Association of Avian Pathologists, Kennett Square, Pa., 1998) pp. 255-266.
184. Timoney, J. F., and Groschup, M. M. (1993) Properties of a protective protein antigen of Erysipelothrix rhusiopathiae. Vet Microbiol. 37, 381-7.
185. Tollis, M., and Di Trani, L. (2002). Recent developments in avian influenza research: epidemiology and immunoprophylaxis. Vet J 164, 202-215.
186. Tooze, J. (1980) DNA Tumor Viruses (Part 2): Molecular Biology of Tumor Viruses, Cold Spring Harbor Laboratory Press, Cold Spring Harbor, N.Y.
187. Toro H, Fernandez I. Avian infectious bronchitis: specific lachrymal IgA level and resistance against challenge. Zentralbl. Veterinarmed B. 1994; 41: 467-472.
188. Toro H, Reyes E, Redmann T, Kaleta E F. Local and systemic specific antibody response of different chicken lines after ocular vaccination against infectious bronchitis. Zentralbl. Veterinarmed. B. 1996; 43:449-454.
189. Toro, H., Tang, D. C., Suarez, D. L., Sylte, M. J., Pfeiffer, J. and Van Kampen, K. R. (2007) Protective avian influenza in ovo vaccination with non-replicating human adenovirus vector. Vaccine 25, 2886-2891.
190. Toro, H., Tang, D. C., Suarez, D. L., Zhang, J., and Shi, Z. (2008) Protection of chickens against avian influenza with non-replicating adenovirus-vectored vaccine. Vaccine 26, 2640-2646.
191. Tsvetkov, T., and Brankova, R. (1983) Viability of micrococci and lactobacilli upon freezing and freeze-drying in the presence of different cryoprotectants. Cryobiology 20, 318-23.
192. Ungchusak, K., Auewarakul, P., Dowell, S. F., Kitphati, R., Auwanit, W., Puthavathana, P., Uiprasertkul, M., Boonnak, K., Pittayawonganon, C., Cox, N. J., et al. (2005). Probable person-to-person transmission of avian influenza A (H5N1). N Engl J Med 352, 333-340.
193. van Ginkel F W, Liu C-J, Simecka J W, Dong J-Y, Greenway T, Frizzell R A, et al. Intratracheal gene delivery with adenoviral vector induces elevated systemic IgG and mucosal IgA antibodies to adenovirus and p-galactosidase. Hum. Gene Ther. 1995; 6: 895-903.
194. Van Kampen, K. R., Shi, Z., Gao, P., Zhang, J., Foster, K. W., Chen, D. T., Marks, D., Elmets, C. A., and Tang, D. C. (2005). Safety and immunogenicity of adenovirus-vectored nasal and epicutaneous influenza vaccines in humans. Vaccine 23, 1029-1036.
195. Vanrompay, D., Cox, E., Volckaert, G., and Goddeeris, B. (1999) Turkeys are protected from infection with Chlamydia psittaci by plasmid DNA vaccination against the major outer membrane protein. Clin Exp Immunol. 118, 49-55.
196. Veits, J., Mettenleiter, T. C., and Fuchs, W. (2003) Five unique open reading frames of infectious laryngotracheitis virus are expressed during infection but are dispensable for virus replication in cell culture. J Gen Virol. 84, 1415-25.
197. Wakenell, P. S., Bryan, T., Schaeffer, J., Avakian, A., Williams, C., and Whitfill, C. (2002). Effect of in ovo vaccine delivery route on herpesvirus of turkeys/SB-1 efficacy and viremia. Avian Dis 46, 274-280.
198. Wang, T. T., Cheng, W. C., and Lee, B. H. (1998) A simple program to calculate codon bias index. Mol. Biotechnol. 10, 103-6.
199. Ward, A. C., Castelli, L. A., Lucantoni, A. C., White, J. F., Azad, A. A., and Macreadie, I. G. (1995) Expression and analysis of the NS2 protein of influenza A virus. Arch. Virol. 140, 2067-2073.
200. Watanabe H, Kobayashi K. Peculiar secretory IgA system identified in chickens. J. Immunol. 1974; 113:1405-1409.
201. Watanabe H, Kobayashi K, Isayama Y. Peculiar Secretory IgA System Identified in Chickens II. Identification and Distribution of Free Secretory Component and Immunoglobulins of IgA, IgM, and IgG in Chicken External Secretions. J. Immunol. 1975; 115: 998-1001.
202. Webby, R. J., Perez, D. R., Coleman, J. S., Guan, Y., Knight, J. H., Govorkova, E. A., McClain-Moss, L. R., Peiris, J. S., Rehg, J. E., Tuomanen, E. I., and Webster, R. G. (2004). Responsiveness to a pandemic alert: use of reverse genetics for rapid development of influenza vaccines. Lancet 363, 1099-1103.
203. Webster, R. G., and Laver, W. G. (1975) Antigenic variation of influenza viruses, P. 270-314. In E. D. Kilbourne (ed.), The influenza viruses and influenza. Academic Press, Inc., New York, N.Y.
204. Webster, R. G., Yakhno, M. A., Hinshaw, V. S., Bean, W. J., and Murti, K. G. (1978) Intestinal influenza: replication and characterization of influenza viruses in ducks. Virology 84, 268-278.
205. Webster, R. G., Laver, W. G., Air, G. M., and Schild, G. C. (1982) Molecular mechanisms of variation in influenza viruses. Nature 296, 115-121.
206. Webster, R. G., and Kawaoka, Y. (1988) Avian influenza. Crit. Rev. Poult. Biol. 1, 211-246.
207. Webster, R. G., Reay, P. A., and Laver, W. G. (1998) Protection against lethal influenza with neuraminidase. Virology 164, 230-237.
208. Wickham T J, Mathias P, Cheresh D A, Nemerow G R. Integrins αᵥβ₃ and αᵥβ₅ promote adenovirus internalization but not virus attachment. Cell 1993; 73:309-319.
209. Wieland W H, Orzáez D, Lammers A, Parmentier H K, Verstegen M W, Schots A. A functional polymeric immunoglobulin receptor in chicken (Gallus gallus) indicates ancient role of secretory IgA in mucosal immunity. Biochem. J. 2004; 380:669-676.
210. Wiersma E J, Collins C, Fazel S, Shulman M J. Structural and functional analysis of J chain-deficient IgM. J. Immunol. 1998; 160:5979-5989.
211. White, E., Denton, A., and Stillman, B. (1988) Role of the adenovirus E1B 19,000-dalton tumor antigen in regulating early gene expression. J. Virol. 62, 3445-54.
212. Widders, P. R., Thomas, L. M., Long, K. A., Tokhi, M. A., Panaccio, M., and Apos, E. (1998) The specificity of antibody in chickens immunised to reduce intestinal colonisation with Campylobacter jejuni. Vet Microbiol. 64, 39-50.
213. Wolff, E., Delisle, B., Corrieu, G., and Gibert, H. (1990) Freeze-drying of Streptococcus thermophilus: a comparison between the vacuum and the atmospheric method. Cryobiology 27, 569-75.
214. Wood, G. W., McCauley, J. W., Bashiruddin, J. B., and Alexander, D. J. (1993) Deduced amino acid sequences at the hemagglutinin cleavage site of avian influenza A viruses of H5 and H7 subtypes. Arch. Virol. 130, 209-217.
215. Wood, G. W., Banks, J., McCauley, J. W., and Alexander, D. J. (1994) Deduced amino acid sequences of the haemagglutinin of H5N1 avian influenza virus isolates from an outbreak in turkeys in Norfolk, England. Arch. Virol. 134, 185-194.
216. Wood, J. M., Major, D., Newman, R. W., Dunleavy, U., Nicolson, C., Robertson, J. S., and Schild, G. C. (2002). Preparation of vaccines against H5N1 influenza. Vaccine 20, S84-S87.
217. Wu C C, Thiagarajan D, Lin T L. Research notes: ELISPOT assay for detection of antibody secreting cells to infectious bursal disease virus in chickens. Poult. Sci. 1998; 77:662-665.
218. Wu, S. Q., Wang, M., Liu, Q., Zhu, Y. J., Suo, X., and Jiang, J. S. (2004) Construction of DNA vaccines and their induced protective immunity against experimental Eimeria tenella infection. Parasitol Res. 94, 332-6.
219. Wyszynska, A., Raczko, A., Lis, M., and Jagusztyn-Krynicka, E. K. (2004) Oral immunization of chickens with avirulent Salmonella vaccine strain carrying C. jejuni 72Dz/92 cjaA gene elicits specific humoral immune response associated with protection against challenge with wild-type Campylobacter. Vaccine 22, 1379-89.
220. Yamaguchi, T., Iritani, Y., and Hayashi, Y. (1988) Serological response of chickens either vaccinated or artificially infected with Haemophilus paragallinarum. Avian Dis. 32, 308-12.
221. Yasuda, J., Nakada, S., Kato, A., Toyoda, T., and Ishihama, A., (1993) Molecular assembly of influenza virus: association of the NS2 protein with virion matrix. Virology 196, 249-255.
222. York, J. J., Strom, A. D., Connick, T. E., McWaters, P. G., Boyle, D. B., and Lowenthal, J. W. (1996) In vivo effects of chicken myelomonocytic growth factor: delivery via a viral vector. J. Immunol. 156, 2991-7.
223. Yoshida, S., Lee, L. F., Yanagida, N., and Nazerian, K. (1994) Identification and characterization of a Marek's disease virus gene homologous to glycoprotein L of herpes simplex virus. Virology 204, 414-9.
224. Young, J. F., and Palese, P. (1979) Evolution of human influenza A viruses in nature. Recombination contributes to genetic variation of H1N1 strains. Proc. Natl. Acad. Sci. USA 76, 6547-6551.
225. Zebedeem S. L., and Lamb, R. A. (1988) Influenza A virus M2 protein: monoclonal antibody restriction of virus growth and detection of M2 in virions. J. Virol. 62, 2762-2772.
226. Zeng, M., Smith, S. K., Siegel, F., Shi, Z., Van Kampen, K. R., Elmets, C. A., and Tang, D. C. (2001). AdEasy system made easier by selecting the viral backbone plasmid preceding homologous recombination. Biotechniques 31, 260-262.

What is claimed is:

1. A method of inducing an immune response in an animal, comprising administering an effective amount of an immunogenic or vaccine composition such that a protective immune response is induced in the animal, wherein the administration is via aerosol spray.

2. The method of claim 1, wherein immunogenic or vaccine composition comprises a recombinant human adenovirus expression vector that comprises and expresses an adenoviral DNA sequence, and a promoter sequence operably linked to a foreign sequence encoding one or more avian antigens or immunogens of interest.

3. The method according to claim 2, wherein the adenoviral DNA sequence is derived from adenovirus serotype 5 (Ad5).

4. The method according to claim 2, wherein the adenoviral DNA sequence is selected from the group consisting of replication-defective adenovirus, non-replicating adenovirus, replication-competent adenovirus, and wild-type adenovirus.

5. The method according to claim 4, wherein the adenoviral DNA sequence is E1/E3 deleted.

6. The method according to claim 2, wherein the promoter sequence is selected from the group consisting of viral promoters, avian promoters, CMV promoter, SV40 promoter, β-actin promoter, albumin promoter, Elongation Factor 1-α (EF 1-α) promoter, PγK promoter, MFG promoter, and Rous sarcoma virus promoter.

7. The method according to claim 2, wherein the foreign sequence encoding the one or more avian antigens or immunogens of interest is derived from avian influenza virus, infectious bursal disease virus, Marek's disease virus, avian herpesvirus, infectious laryngotracheitis virus, avian infectious bronchitis virus, avian reovirus, avipox, fowlpox, canarypox, pigeonpox, quailpox, and dovepox, avian polyomavirus, Newcastle Disease virus, avian pneumovirus, avian rhinotracheitis virus, avian reticuloendotheliosis virus, avian retroviruses, avian endogenous virus, avian erythroblastosis virus, avian hepatitis virus, avian anemia virus, avian enteritis virus, Pacheco's disease virus, avian leukemia virus, avian parvovirus, avian rotavirus, avian leukosis virus, avian musculoaponeurotic fibrosarcoma virus, avian myeloblastosis virus, avian myeloblastosis-associated virus, avian myelocytomatosis virus, avian sarcoma virus, or avian spleen necrosis virus.

8. The method according to claim 7, wherein the foreign sequence encoding the one or more avian antigens or immunogens of interest is derived from one or more avian viruses.

9. The method according to claim 7, wherein the foreign sequence encoding the one or more avian antigens or immunogens of interest is derived from avian influenza virus.

10. The method of claim 7, wherein the foreign sequence encoding the one or more avian antigens or immunogens of interest is selected from the group consisting of genes encoding hemagglutinin, spike protein, other external proteins, nucleoprotein, matrix, neuraminidase, and non-structural proteins such as enzymes and other regulatory proteins.

11. The method of claim 7, wherein the foreign sequence encoding the one or more avian antigens or immunogens of interest is selected from the group consisting of hemagglutinin subtype 3, 5, 7, and 9.

12. An immunogenic composition or vaccine for aerosol spray delivery to an avian subject comprising a veterinarily acceptable vehicle or excipient and a recombinant human adenovirus expression vector that comprises and expresses an adenoviral DNA sequence, and a promoter sequence operably linked to a foreign sequence encoding one or more avian antigens or immunogens of interest.

13. The immunogenic composition or vaccine of claim 12, wherein the adenoviral DNA sequence is derived from adenovirus serotype 5 (Ad5).

14. The immunogenic composition or vaccine of claim 12, wherein the adenoviral DNA sequence is selected from the group consisting of replication-defective adenovirus, non-replicating adenovirus, replication-competent adenovirus, and wild-type adenovirus.

15. The immunogenic composition or vaccine of claim 14, wherein the adenoviral DNA sequence is E1/E3 deleted.

16. The immunogenic composition or vaccine of claim 12, wherein the promoter sequence is selected from the group consisting of viral promoters, avian promoters, CMV promoter, SV40 promoter, β-actin promoter, albumin promoter, Elongation Factor 1-α (EF1-α) promoter, PγK promoter, MFG promoter, and Rous sarcoma virus promoter.

17. The immunogenic composition or vaccine of claim 12, wherein the foreign sequence encoding the one or more avian antigens or immunogens of interest is derived from avian influenza virus, infectious bursal disease virus, Marek's disease virus, avian herpesvirus, infectious laryngotracheitis virus, avian infectious bronchitis virus, avian reovirus, avipox, fowlpox, canarypox, pigeonpox, quailpox, and dovepox, avian polyomavirus, Newcastle Disease virus, avian pneumovirus, avian rhinotracheitis virus, avian reticuloendotheliosis virus, avian retroviruses, avian endogenous virus, avian erythroblastosis virus, avian hepatitis virus, avian anemia virus, avian enteritis virus, Pacheco's disease virus, avian leukemia virus, avian parvovirus, avian rotavirus, avian leukosis virus, avian musculoaponeurotic fibrosarcoma virus, avian myeloblastosis virus, avian myeloblastosis-associated virus, avian myelocytomatosis virus, avian sarcoma virus, or avian spleen necrosis virus.

18. The immunogenic composition or vaccine of claim 12, wherein the foreign sequence encoding the one or more avian antigens or immunogens of interest is derived from one or more avian viruses.

19. The immunogenic composition or vaccine of claim 12, wherein the foreign sequence encoding the one or more avian antigens or immunogens of interest is derived from avian influenza virus.

20. The immunogenic composition or vaccine of claim 19, wherein the foreign sequence encoding the one or more avian antigens or immunogens of interest is selected from the group consisting of genes encoding hemagglutinin, spike protein, other external proteins, nucleoprotein, matrix, neuraminidase, and non-structural proteins such as enzymes and other regulatory proteins.

21. The immunogenic composition or vaccine of claim 20, wherein the foreign sequence encoding the one or more avian antigens or immunogens of interest is selected from the group consisting of hemagglutinin subtype 3 and 5.

22. The composition or vaccine of claim 12, further comprising an adjuvant.

23. The composition or vaccine of claim 12, further comprising an additional vaccine.

24. A method of eliciting an immunogenic response to avian influenza in an avian subject, comprising administering an immunologically effective amount of the composition of claim 12 to the avian subject.

25. Method of eliciting an immunogenic response in an avian subject, comprising infecting the avian subject with an immunologically effective amount of an immunogenic composition comprising a recombinant human adenovirus expression vector that comprises and expresses an adenoviral DNA sequence, and a promoter sequence operably linked to a foreign sequence encoding one or more avian antigens or immunogens of interest, wherein the one or more avian antigens or immunogens of interest are expressed at a level sufficient to elicit an immunogenic response to the one or more avian antigens or immunogens of interest in the avian subject, wherein the immunogenic composition is administered via aerosol spray.

26. The method of claim 25, wherein the one or more avian antigens or immunogens of interest are derived from avian influenza virus, infectious bursal disease virus, Marek's disease virus, avian herpesvirus, infectious laryngotracheitis virus, avian infectious bronchitis virus, avian reovirus, avipox, fowlpox, canarypox, pigeonpox, quailpox, and dovepox, avian polyomavirus, Newcastle Disease virus, avian pneumovirus, avian rhinotracheitis virus, avian reticuloendotheliosis virus, avian retroviruses, avian endogenous virus, avian erythroblastosis virus, avian hepatitis virus, avian anemia virus, avian enteritis virus, Pacheco's disease virus, avian leukemia virus, avian parvovirus, avian rotavirus, avian leukosis virus, avian musculoaponeurotic fibrosarcoma virus, avian myeloblastosis virus, avian myeloblastosis-associated virus, avian myelocytomatosis virus, avian sarcoma virus, or avian spleen necrosis virus.

27. The method of claim 25, wherein the foreign sequence encoding the one or more avian antigens or immunogens of interest is derived from avian influenza virus.

28. The method of claim 27, wherein the foreign sequence encoding the one or more avian antigens or immunogens of interest is selected from the group consisting of genes encoding hemagglutinin, spike protein, other external proteins, nucleoprotein, matrix, neuraminidase, and non-structural proteins such as enzymes and other regulatory proteins.

29. The method of claim 28, wherein the foreign sequence encoding the one or more avian antigens or immunogens of interest is selected from the group consisting of hemagglutinin subtype 3, 5, 7, and 9.

30. The method of claim 25, further comprising an additional vaccine.

31. A method for inoculation of an avian subject, comprising aerosol spray administration of a recombinant human adenovirus containing and expressing an heterologous nucleic acid molecule encoding an antigen of a pathogen of the avian subject.

32. The method of claim 31, wherein the human adenovirus comprises sequences derived from adenovirus serotype 5.

33. The method of claim 31, wherein the human adenovirus comprises sequences derived from replication-defective adenovirus, non-replicating adenovirus, replication-competent adenovirus, or wild-type adenovirus.

34. The method of claim 33, wherein the adenoviral DNA sequence is E1/E3 deleted.

35. The method of claim 31, wherein the antigen of a pathogen of the avian is derived from avian influenza virus, infectious bursal disease virus, Marek's disease virus, avian herpesvirus, infectious laryngotracheitis virus, avian infectious bronchitis virus, avian reovirus, avipox, fowlpox, canarypox, pigeonpox, quailpox, and dovepox, avian polyomavirus, Newcastle Disease virus, avian pneumovirus, avian rhinotracheitis virus, avian reticuloendotheliosis virus, avian retroviruses, avian endogenous virus, avian erythroblastosis virus, avian hepatitis virus, avian anemia virus, avian enteritis virus, Pacheco's disease virus, avian leukemia virus, avian parvovirus, avian rotavirus, avian leukosis virus, avian musculoaponeurotic fibrosarcoma virus, avian myeloblastosis virus, avian myeloblastosis-associated virus, avian myelocytomatosis virus, avian sarcoma virus, or avian spleen necrosis virus.

36. The method of claim 35, wherein the antigen of a pathogen of the avian is derived from avian influenza virus.

37. The method of claim 36, wherein the foreign sequence encoding the one or more avian antigens or immunogens of interest is selected from the group consisting of genes encoding hemagglutinin, spike protein, other external proteins, nucleoprotein, matrix, neuraminidase, and non-structural proteins such as enzymes and other regulatory proteins.

38. The method of claim 36, wherein the avian influenza antigens or immunogens are selected from the group consisting of hemagglutinin subtype 3, 5, 7, and 9.

39. The method of claim 31, further comprising administering an additional vaccine.

40. An aerosol spray administration apparatus for delivery of an immunogenic composition to one or more avians wherein the apparatus contains a recombinant human adenovirus expression vector expressing one or more avian antigens or immunogens of interest, wherein the apparatus delivers to the recombinant human adenovirus to the one or more avians.

41. The apparatus of claim 40, wherein the human adenovirus expression vector comprises sequences derived from adenovirus serotype 5.

42. The apparatus of claim 40, wherein the human adenovirus expression vector comprises sequences derived from replication-defective adenovirus, non-replicating human adenovirus, replication-competent adenovirus, or wild-type adenovirus.

43. The apparatus of claim 40, wherein the adenoviral DNA sequence is E1/E3 deleted.

44. The apparatus of claim 40, wherein the one or more avian antigens or immunogens of interest are derived from avian influenza virus, infectious bursal disease virus, Marek's disease virus, avian herpesvirus, infectious laryngotracheitis virus, avian infectious bronchitis virus, avian reovirus, avipox, fowlpox, canarypox, pigeonpox, quailpox, and dovepox, avian polyomavirus, Newcastle Disease virus, avian pneumovirus, avian rhinotracheitis virus, avian reticuloendotheliosis virus, avian retroviruses, avian endogenous virus, avian erythroblastosis virus, avian hepatitis virus, avian anemia virus, avian enteritis virus, Pacheco's disease virus, avian leukemia virus, avian parvovirus, avian rotavirus, avian leukosis virus, avian musculoaponeurotic fibrosarcoma virus, avian myeloblastosis virus, avian myeloblastosis-associated virus, avian myelocytomatosis virus, avian sarcoma virus, or avian spleen necrosis virus.

45. The method of claim 40, wherein the one or more avian antigens or immunogens of interest are derived from avian influenza virus.

46. The method of claim 40, wherein the foreign sequence encoding the one or more avian antigens or immunogens of interest is selected from the group consisting of genes encoding hemagglutinin, spike protein, other external proteins, nucleoprotein, matrix, neuraminidase, and non-structural proteins such as enzymes and other regulatory proteins.

47. The method of claim 40, wherein the avian influenza antigens or immunogens of interest is selected from the group consisting of hemagglutinin subtype 3, 5, 7, and 9.

48. The method of claim 40 further comprising administering an additional vaccine.


SEQUENCE LISTING (14 sequences)

SEQ ID NO: 1 - DNA, 1701 bp, Influenza A virus
  CDS (1)..(1698)
atgaagactatcattgctttgagctacattttatgtctggttttcgctcaaaaacttcccggaaatgacaacagcacggcaacgctgtgcctggggcaccatgcagtgtcaaacggaacgctagtgaaaacaatcacgaatgaccaaattgaagtgactaatgctactgagctggttcagagttcctcaacaggtagaatatgcgacagtcctcaccaaatccttgatggagaaaactgcacactaatagatgctctattgggagaccctcattgtgatggcttccaaaataaggaatgggacctttttgttgaacgcagcaaagcctacagcaactgttacccttatgatgtgccggattatgcctcccttaggtcactagttgcctcatccggcacactggagtttaacaatgaaagcttcaattggactggagtcgctcagaatggaacaagctctgcttgcaaaaggagatctaataaaagtttctttagtagattgaattggttgcaccaattaaaatacaaatatccagcactgaacgtgactatgccaaacaatgaaaaatttgacaaattgtacatttggggggttcaccacccgagtacggacagtgaccaaatcagcatatatgctcaagcatcagggagagtcacagtctctaccaaaagaagccaacaaactgtaatcccgaatatcggatctagtccctgggtaaggggtgtctccagcagaataagcatctattggacaatagtaaaaccgggagacatacttttgattaacagcacagggaatctaattgctcctcggggttacttcaaaatacgaagtgggaaaagctcaataatgaggtcagatgcacccattggcaaatgcaattctgaatgcatcactccaaatggaagcattcccaatgacaaaccatttcaaaatgtaaacaggatcacatatggggcctgtcccagatatgttaagcaaaacactctgaaattggcaacagggatgcggaatgtaccagagaaacaaactagaggcatattcggcgcaatcgcgggtttcatagaaaatggttgggagggaatggtggacggttggtacggtttcaggcatcaaaattctgagggcacaggacaagcagcagatcttaaaagcactcaagcagcaatcaaccaaatcaacgggaaactgaataggttaatcgagaaaacgaacgagaaattccatcaaattgaaaaagaattctcagaagtagaagggagaattcaggacctcgagaaatatgttgaggacactaaaatagatctctggtcgtacaacgcggagcttcttgttgccctggagaaccaacatacaattgatctaactgactcagaaatgaacaaactgtttgaaagaacaaagaagcaactgagggaaaatgctgaggatatgggcaatggttgtttcaaaatataccacaaatgtgacaatgcctgcatagggtcaatcagaaatggaacttatgaccatgatgtatacagagacgaagcattaaacaaccggttccagatcaaaggtgttgagctgaagtcaggatacaaagattggatcctatggatttcctttgccatatcatgctttttgctttgtgttgttttgctggggttcatcatgtgggcctgccaaaaaggcaacattaggtgcaacatttgcatttga

SEQ ID NO: 2 - protein, 566 aa, Influenza A virus
MKTIIALSYILCLVFAQKLPGNDNSTATLCLGHHAVSNGTLVKTITNDQIEVTNATELVQSSSTGRICDSPHQILDGENCTLIDALLGDPHCDGFQNKEWDLFVERSKAYSNCYPYDVPDYASLRSLVASSGTLEFNNESFNWTGVAQNGTSSACKRRSNKSFFSRLNWLHQLKYKYPALNVTMPNNEKFDKLYIWGVHHPSTDSDQISIYAQASGRVTVSTKRSQQTVIPNIGSSPWVRGVSSRISIYWTIVKPGDILLINSTGNLIAPRGYFKIRSGKSSIMRSDAPIGKCNSECITPNGSIPNDKPFQNVNRITYGACPRYVKQNTLKLATGMRNVPEKQTRGIFGAIAGFIENGWEGMVDGWYGFRHQNSEGTGQAADLKSTQAAINQINGKLNRLIEKTNEKFHQIEKEFSEVEGRIQDLEKYVEDTKIDLWSYNAELLVALENQHTIDLTDSEMNKLFERTKKQLRENAEDMGNGCFKIYHKCDNACIGSIRNGTYDHDVYRDEALNNRFQIKGVELKSGYKDWILWISFAISCFLLCVVLLGFIMWACQKGNIRCNICI

SEQ ID NO: 3 - DNA, 1647 bp, Influenza A virus
  CDS (1)..(1644)
gaccaaatctgcatcggttatcatgcaaacaattcaacaaaacaagttgacacaatcatggagaagaatgtgacggtcacacatgctcaagatatactggaaaaagagcacaacgggaaactctgcagtctcaaaggagtgaggcccctcattctgaaggattgcagtgtggctggatggcttcttgggaacccaatgtgtgatgagttcctaaatgtaccggaatggtcatatattgtagagaaggacaatccaaccaatggcttatgttatccgggagacttcaatgattatgaagaactgaagtatttaatgagcaacacaaaccattttgagaaaattcaaataatccctaggaactcttggtccaatcatgatgcctcatcaggagtgagctcagcatgcccatacaatggtaggtcttcctttttcaggagtgtggtgtggttgatcaagaagagtaatgtatacccaacaataaagaggacctacaataacaccaatgtagaggaccttctgatattgtggggaatccatcaccctaatgatgcagcggaacaaacggaactctatcagaactcgaacacttatgtgtctgtaggaacatcaacactaaatcagaggtcaattccagaaatagctaccaggcccaaagtgaatggacaaagtggaagaatagaatttttctggacaatactaaggccgaacgatgcaatcagctttgaaagtaatgggaactttatagctcctgaatatgcatacaagatagttaaaaagggagattcagcaatcatgagaagcgaactggagtatggcaactgtgataccaaatgtcagaccccagtgggtgctataaattccagtatgccttttcacaatgttcatccccttaccattggagagtgtcccaaatatgtcaaatcagataaactggtccttgcaacaggactgaggaacgtgcctcagagagaaacaagaggtctgtttggagcaatagcaggattcatagaaggggggtggcaaggaatggtagatggatggtatggttaccatcatagcaacgagcagggaagtggatatgctgcagacaaagagtccactcagaaagcaatcgacgggatcaccaataaagtcaactcaatcattgacaaaatgaacactcaattcgaagccgttgggaaagaattcaacaacttagaaaggagaatagaaaatttgaataagaaaatggaagatggatttctagatgtatggacttacaatgcagaacttctggtgctcatggaaaatgaaagaactctggatttccatgattcatatgtcaagaacctatacgataaggtccgactccagctgagagataatgcaaaagaattgggcaatgggtgtttggagttctcccacaaatgtgacaatgaatgcatggaaagtgtgagaaacggaacgtatgactatccacaatactcagaagaatcaaggctgaacagagaggaaatagatggagtcaaattggagtcaatgggcacctatcagatactatcaatttactcaacagtggcgagttccctagcactggcaatcatggtagctggtctgtctttttggatgtgctccaatggatcattgcaatgcagaatttgcatctag

SEQ ID NO: 4 - protein, 548 aa, Influenza A virus
DQICIGYHANNSTKQVDTIMEKNVTVTHAQDILEKEHNGKLCSLKGVRPLILKDCSVAGWLLGNPMCDEFLNVPEWSYIVEKDNPTNGLCYPGDFNDYEELKYLMSNTNHFEKIQIIPRNSWSNHDASSGVSSACPYNGRSSFFRSVVWLIKKSNVYPTIKRTYNNTNVEDLLILWGIHHPNDAAEQTELYQNSNTYVSVGTSTLNQRSIPEIATRPKVNGQSGRIEFFWTILRPNDAISFESNGNFIAPEYAYKIVKKGDSAIMRSELEYGNCDTKCQTPVGAINSSMPFHNVHPLTIGECPKYVKSDKLVLATGLRNVPQRETRGLFGAIAGFIEGGWQGMVDGWYGYHHSNEQGSGYAADKESTQKAIDGITNKVNSIIDKMNTQFEAVGKEFNNLERRIENLNKKMEDGFLDVWTYNAELLVLMENERTLDFHDSYVKNLYDKVRLQLRDNAKELGNGCLEFSHKCDNECMESVRNGTYDYPQYSEESRLNREEIDGVKLESMGTYQILSIYSTVASSLALAIMVAGLSFWMCSNGSLQCRICI

SEQ ID NO: 5 - DNA, 1659 bp, Influenza A virus
  CDS (1)..(1656)
gaccaaatctgcattggttatcatgcaaacaattcaacaaaacaggttgacacaatcatggagaagaatgtgacggtcacacatgctcaggacatactggaaaaagaacacaatggaagactctgcagtcttaaaggagtgaagcccctcattctgaaggattgcagtgtagctggatggcttcttggaaatccaatgtgtgatgaattcctgaatgtaccggaatggtcatatattgtggaaaaggacaatccagccaatggcctgtgttatccgggaaacttcaacgattatgaagaactgaagcatttaatgagcagcacaaaccattttgagaaaattcagatatttcctaggagctcttggtccaaccatgatgcctcatcaggagtgagctctgcatgcccatacaatggtaggtcttcctttttcaggaatgtagtgtggctgatcaagaagaataatgtgtaccgaacaataaagaggacctaccataacactaatgtagaagaccttttaatattatggggaattcatcaccctaatgatgcagctgaacagataaaactctaccagaacccgaacacttacgtgtcagtgggaacatcaacattgaatcaaaggtcaatcccagaaatagccaccagacccaaggtgaacggacagagtggaaggatggaatttttttggacaatactaaggccgaacgactcaatcaactttgagagtactgggaactttatagctcctgaatatgcatacaagcttattaaaaaaggagattcagcaatcatgaaaagtgaactgaattatggtaactgtgataccaaatgtcagaccccagcgggtgctataaattccaggatgccttttcacaatgtccatccttttactattggggagtgccccaagtatgtcaaatcgaaaaaactagttcttgcaacagggctaagaaacgtaccccaaagaaaaagaaaaagaaaaacaagaggcctatttggagcaatagccggattcatagaaggaggatggcaaggaatggtggatggatggtatggatatcatcatagcaatgagcagggaagtggatatggtgaagacaacgaatctacacagaaagcaatcgatgggatcactaataaagtcaactcaatcattgacaaaatgaacactcaattcgaagccgttgggaaagaattcaacaacctagaaaggagaatagaaaatttgaataagaaaatggaagatggctttatagatgtatggacttacaatgcggaacttctagtgctcatggaaaacgaaagaactctggatctccatgattcaaatgtcaagaaattatacgatagggtccgactccagctgagagacaatgccaaagaattaggcaatgggtgctttgaattctaccacaagtgtgacaatgaatgcatggaaagtgtgagaaatggaacgtatgactatccacaatactcagaagaatcaagactgaacagggaggaaatagacggagtcaaattagaatcaatggggacttatcagatactttcaatctattcaacagtagcgagttccctagcactggcaatcatggtagctggtctatctttttggatgtgttccaatggatcattacagtgcagaatttgcatctag

SEQ ID NO: 6 - protein, 552 aa, Influenza A virus
DQICIGYHANNSTKQVDTIMEKNVTVTHAQDILEKEHNGRLCSLKGVKPLILKDCSVAGWLLGNPMCDEFLNVPEWSYIVEKDNPANGLCYPGNFNDYEELKHLMSSTNHFEKIQIFPRSSWSNHDASSGVSSACPYNGRSSFFRNVVWLIKKNNVYRTIKRTYHNTNVEDLLILWGIHHPNDAAEQIKLYQNPNTYVSVGTSTLNQRSIPEIATRPKVNGQSGRMEFFWTILRPNDSINFESTGNFIAPEYAYKLIKKGDSAIMKSELNYGNCDTKCQTPAGAINSRMPFHNVHPFTIGECPKYVKSKKLVLATGLRNVPQRKRKRKTRGLFGAIAGFIEGGWQGMVDGWYGYHHSNEQGSGYGEDNESTQKAIDGITNKVNSIIDKMNTQFEAVGKEFNNLERRIENLNKKMEDGFIDVWTYNAELLVLMENERTLDLHDSNVKKLYDRVRLQLRDNAKELGNGCFEFYHKCDNECMESVRNGTYDYPQYSEESRLNREEIDGVKLESMGTYQILSIYSTVASSLALAIMVAGLSFWMCSNGSLQCRICI

SEQ ID NO: 7 - DNA, 1647 bp, Influenza A virus; identical to SEQ ID NO: 3
  CDS (1)..(1644)

SEQ ID NO: 8 - protein, 548 aa, Influenza A virus; identical to SEQ ID NO: 4

SEQ ID NO: 9 - DNA, 39 bp, Artificial Sequence
  source: /note=“Description of Artificial Sequence Synthetic primer”
cacacaggtaccgccatgaagactatcattgctttgagc

SEQ ID NO: 10 - DNA, 32 bp, Artificial Sequence
  source: /note=“Description of Artificial Sequence Synthetic primer”
cacacaggtacctcaaatgcaaatgttgcacc

SEQ ID NO: 11 - DNA, 36 bp, Artificial Sequence
  source: /note=“Description of Artificial Sequence Synthetic primer”
cacacaggatccatctgaactcacaatcctagatgc

SEQ ID NO: 12 - DNA, 38 bp, Artificial Sequence
  source: /note=“Description of Artificial Sequence Synthetic primer”
cacacaaagcttgccgccatggaaagaatagtgattgc

SEQ ID NO: 13 - DNA, 19 bp, Artificial Sequence
  source: /note=“Description of Artificial Sequence Synthetic primer”
tgtcagttcgttgaggacc

SEQ ID NO: 14 - DNA, 20 bp, Artificial Sequence
  source: /note=“Description of Artificial Sequence Synthetic primer”
gttccctcttaggacgactc